SEQUENCE LISTING (18 sequences)

SEQ ID NO: 1 - DNA, 375 bp, Homo sapiens
  source 1..375: mol_type = other DNA
caggtgcagctggtggagtctgggggaggcgtggtccagcctgggaggtccctgagactctcctgtgcagcctctggattcaccttcagtagtcatgttatgtactgggtccgccaggctccaggcaaggggctggagtgggtggcagttataacatatgatggaggcaataaatactacgcagactccgtgaggggccgactcaccatctccagagacaattccaagaacacactgtatctgcaaatgaacagcctgagagctgaggacacggctgtgtattactgtgcgagtccccgtggggatagtgggagctactacgatatagactactttgactactggggccagggaaccctggtcaccgtctcctcg

SEQ ID NO: 2 - DNA, 321 bp, Homo sapiens
  source 1..321: mol_type = other DNA
gacatccagatgacccagtctccatctgccatgtctgcatctgtaggagacagagtcaccatcacttgtcgggcgagtcagggcattagcaattatttagcctggtttcagcagaaaccagggaaagtccctaagcgcctgatctatgctgcatccagtttgcaaagtggggtcccatcaaggttcagcggcagtggatctgggacagaattcactctcacaatcagcagcctgcagcctgaagattttgcaacttattactgtctacagcataatacttccccttccccgttcggccaagggaccaaggtggaaatcaaa

SEQ ID NO: 3 - protein, 125 aa, Homo sapiens
QVQLVESGGGVVQPGRSLRLSCAASGFTFSSHVMYWVRQAPGKGLEWVAVITYDGGNKYYADSVRGRLTISRDNSKNTLYLQMNSLRAEDTAVYYCASPRGDSGSYYDIDYFDYWGQGTLVTVSS

SEQ ID NO: 4 - protein, 107 aa, Homo sapiens
DIQMTQSPSAMSASVGDRVTITCRASQGISNYLAWFQQKPGKVPKRLIYAASSLQSGVPSRFSGSGSGTEFTLTISSLQPEDFATYYCLQHNTSPSPFGQGTKVEIK

SEQ ID NO: 5 - protein, 8 aa, Homo sapiens
GFTFSSHV

SEQ ID NO: 6 - protein, 8 aa, Homo sapiens
ITYDGGNK

SEQ ID NO: 7 - protein, 18 aa, Homo sapiens
ASPRGDSGSYYDIDYFDY

SEQ ID NO: 8 - protein, 6 aa, Homo sapiens
QGISNY

SEQ ID NO: 9 - skipped

SEQ ID NO: 10 - protein, 9 aa, Homo sapiens
LQHNTSPSP

SEQ ID NO: 11 - DNA, 29 bp, synthetic construct
  source 1..29: mol_type = other DNA
atgtgcgtcctggccaatatcacgtttcc

SEQ ID NO: 12 - DNA, 30 bp, synthetic construct
  source 1..30: mol_type = other DNA
gaacaaaactagggcaaccactgctgtagc

SEQ ID NO: 13 - protein, 419 aa, Homo sapiens
DLDTHFTQYKLARPYIADCPNCGHSRCDSPIAIEEVRGDAHAGVIRIQTSAMFGLKTDGVDLAYMSFMNGKTQKSIKIDNLHVRTSAPCSLVSHHGYYILAQCPPGDTVTVGFHDGPNRHTCTVAHKVEFRPVGREKYRHPPEHGVELPCNRYTHKRADQGHYVEMHQPGLVADHSLLSIHSAKVKITVPSGAQVKYYCKCPDVREGITSSDHITTCTDVKQCRAYLINKKWVYNSGRLPRGEGDTFKGKLHVPFVPVKAKCIATLAPEPLVEHKHRTLILHLHPDHPTLLTTRSLGSDANPTRQWIERPTTVNFTVTGEGLEYTWGNHPPKRVWAQESGEGNPHGWPHEVVVYYYNRYPLTTIIGLCTCVAIIMVSCVTSVWLLCRTRNLCITYPKLAPNAQVPILLALLCCIKPTRA

SEQ ID NO: 14 - protein, 423 aa, Homo sapiens
SITDDFTLTSPYLGFCPYCRHSAPCFSPIKIENVWDESDDGSIRIQVSAQFGYNQAGTADVTKFRYMSYDHDHDIKEDSMEKIAISTSGPCRRLGHKGYFLLAQCPPGDSVTVSITSGASENSCTVEKKIRRKFVGREEYLFPPVQGKLVKCHVYDRLKETSAGYITMHRPGPHAYKSYLKEASGEVYIKPPSGKNVTYECKCGDYSTGIVSTQTKMNGCTKARQCIAYKLDQTKWVFNSPDLIRHTDHSVQGKLHIPFRLTPTVCPVPLAHTPTVTKWFKGITLHLTATRPTLLTTRKLGLRADATAEWITGTTSRNFSVGREGLEYVWGNHEPVRVWAQESAPGDPHGWPHEIIIHYYHRHPVYTVIVLCGVALAILVGTASSAACIAKARRDCLTPYALAPNATVPTALAVLCCIRPTNA

SEQ ID NO: 15 - protein, 423 aa, Homo sapiens
STEELFKEYKLTRPYMARCIRCAVGSCHSPIAIEAVKSDGHDGYVRLQTSSQYGLDSSGNLKGRTMRYDMHGTIKEIPLHQVSLHTSRPCHIVDGHGYFLLARCPAGDSITMEFKKDSVTHSCSVPYEVKFNPVGRELYTHPPEHGVEQACQVYAHDAQNRGAYVEMHLPGSEVDSSLVSLSGSSVTVTPPVGTSALVECECGGTKISETINKTKQFSQCTKKEQCRAYRLQNDKWVYNSDKLPKAAGATLKGKLHVPFLLADGKCTVPLAPEPMITFGFRSVSLKLHPKNPTYLTTRQLADEPHYTHELISEPAVRNFTVTEKGWEFVWGNHPPKRFWAQETAPGNPHGLPHEVITHYYHRYPMSTILGLSICAAIATVSVAASTWLFCRSRVACLTPYRLTPNARIPFCLAVLCCARTARA

SEQ ID NO: 16 - protein, 421 aa, Homo sapiens
SIKDNFNVYKAIRPYLAHCPDCGEGHSCHSPVALERIRNEATDGTLKIQVSLQIGIKTDDSHWTKLRYMDNHMPADAERARLFVRTSAPCTITGTMGHFILARCPKGETLTVGFTDGRKISHSCTHPFHHDPPVIGREKFHSRPQHGRELPCSTYAQSTAATAEEIEVHMPPDTPDRTLMSQQSGNVKITVNSQTVRYKCNCGDSNEGLTTTDKVINNCKVDQCHAAVTNHKKWQYNSPLVPRAELGDRKGKVHIPFPLANVTCRVPKARNPTVTYGKNQVIMLLYPDHPTLLSYRNMGEEPNYQEEWVTHKKEIRLTVPTEGLEVTWGNNEPYKYWPQLSTNGTAHGHPHEIILYYYELYPTMTVVVVSVASFVLLSMVGVAVGMCMCARRRCITPYELTPGATVPFLLSLTCCIRTAKA

SEQ ID NO: 17 - protein, 422 aa, Homo sapiens
SVTEHFNVYKATRPYLAYCADCGDGYFCYSPVAIEKIRDEAPDGMIKIQVSAQIGLDKAGTHAHTKIRYMAGHDVQESKRDSLRVYTSAACSIHGTMGHFIVAHCPPGDYLKVSFEDADSHVKACKVQYKHDPLPVGREKFVVRPHFGVELPCTSYQLTTAPTDEEIDMHTPPDIPDRTLLSQTAGNVKITAGGRTIRYNCTCGRDNVGTTSTDKTINTCKIDQCHAAVTSHDKWQFTSPFVPRADQTARRGKVHVPFPLTNVTCRVPLARAPDVTYGKKEVTLRLHPDHPTLFSYRSLGAEPHPYEEWVDKFSERIIPVTEEGIEYQWGNNPPVRLWAQLTTEGKPHGWPHEIIQYYYGLYPAATIAAVSGASLMALLTLAATCCMLATARRKCLTPYALTPGAVVPLTLGLLCCAPRANA

SEQ ID NO: 18 - protein, 421 aa, Homo sapiens
STANHFNAYKLTRPYVAYCADCGMGHSCHSPAMIENVQADATDGTLKIQFASQIGLTKKDTHDHTKIRYAEGHDIAEAARSTLKVHSSSECAVTGTMGHFILAKCPPGEVISVSFVDSKNEQRTCTIAYRHEQRLIGRERFTVRPHHGIELPCTTYQLTTAETSEEIDMHMPPDVPDRTILSQQSGNVKITVNGRIVKYSCSCGSKPSGTTTTDKTINSCTVDKCQAYVTSHTKWQFNSPFVPRAEQAERKGKVHIPFPLINTTCRVPLAPEAVRSGKREATLSLHPIHPTLLSYRTLGREPVFDEQWITTQTEVTIPVPVEGVEYRWGNHKPQRLWSQLTTEGRAHGWPHEIIEYYYGLHPTTTIVVVVAVSVVVLLSVAASVYMCVVARNKCLTPYALTPGAVVPVTIGVLCCAPKAHA

(19) United States
(12) Patent Application Publication
(10) Pub. No.: US 2025/0277015 A1
(43) Pub. Date: Sep. 4, 2025

(54) HUMAN MONOCLONAL ANTIBODIES TO EASTERN EQUINE ENCEPHALITIS VIRUS AND USES THEREFOR

(71) Applicant: Vanderbilt University, Nashville, TN (US)

(72) Inventors: James E. CROWE, JR., Nashville, TN (US); Lauren E. WILLIAMSON, Nashville, TN (US)

(73) Assignee: Vanderbilt University, Nashville, TN (US)

(21) Appl. No.: 19/067,564

(22) Filed: Feb. 28, 2025

Related U.S. Application Data
(60) Provisional application No. 63/561,106, filed on Mar. 4, 2024.

(51) Int. Cl. C07K 16/10; A61K 39/00; A61P 31/04; G01N 33/569

(52) U.S. Cl. CPC C07K 16/1081; A61P 31/04; G01N 33/56983; A61K 2039/505; C07K 2317/24; C07K 2317/31; C07K 2317/41; C07K 2317/565; C07K 2317/72; C07K 2317/76; G01N 2333/181

(57) ABSTRACT

The present disclosure is directed to antibodies binding to and neutralizing Eastern Equine Encephalitis Virus (EEEV) and methods for use thereof.

### PRIORITY CLAIM

This application claims benefit of priority to U.S. Provisional Application Ser. No. 63/561,106, filed Mar. 4, 2024, the entire contents of which are hereby incorporated herein by reference.

### STATEMENT REGARDING FEDERALLY FUNDED RESEARCH

This invention was made with government support under grant R01 AI172156 and grant U19 AI142790 awarded by the National Institutes of Health. The government has certain rights in the invention.

### REFERENCE TO A SEQUENCE LISTING

This application contains a Sequence Listing XML, which has been submitted electronically and is hereby incorporated by reference in its entirety. Said XML Sequence Listing, created on Feb. 26, 2025, is named VBLTP0342US.xml and is 19,271 bytes in size.

### BACKGROUND

### 1. Field of the Disclosure

The present disclosure relates generally to the fields of medicine, infectious disease, and immunology. More particularly, the disclosure relates to human antibodies binding to Eastern equine encephalitis virus (EEEV).

### 2. Background

The humoral response to viral infections targets a wide range of epitopes on the virion, including quaternary epitopes¹⁻⁴. Antibody-antigen complexes help elucidate the structural basis for recognition of diverse epitopes by neutralizing antibodies. However, most of these studies used antibody Fab fragments instead of IgG, which limits the understanding of mechanism of action to monovalent antibody-antigen interactions that may inaccurately describe the occupancy of bound sites on virion particles³⁻⁷. A more limited set of studies investigated intact neutralizing antibodies in complex with virion particles through structural analyses^{8,9}.

The encephalitic alphaviruses are mosquito-transmitted viruses and include EEEV, Western (WEEV), and Venezuelan (VEEV) equine encephalitis viruses. EEEV is the most virulent amongst the encephalitic alphaviruses, with a reported human case fatality rate of ˜30% and neurological sequelae in ˜90% of survivors¹⁰⁻¹⁴. In the United States, on average 11 human cases of encephalitic disease caused by EEEV are reported each year (world-wide-web at cdc.gov/easternequineencephalitis/). However, a spike in cases was observed in 2019, in which there were 38 reported human cases and 19 deaths across the United States due to EEEV. Although reported human case numbers decreased in the following years, 19 cases of EEEV neuroinvasive disease were reported in 2024, highlighting the concerns about the potential for larger future EEEV outbreaks.

Unfortunately, there are no FDA-approved vaccines or antiviral treatments against EEEV available for human use. In addition, the encephalitic alphaviruses can be aerosolized for use as a potential bioweapon 15. Given these significant concerns coupled with the lack of prevention or treatment options (vaccines or antiviral drugs) for humans, EEEV is classified as a NIAID Category B priority pathogen and an USDA/CDC Select Agent.

Alphaviruses are enveloped viruses with a positive-sense single-stranded ˜11.7 kb RNA genome that encodes both the non-structural and structural proteins^{6,16,17}. The alphavirus structural polyprotein encodes five to six structural proteins: capsid, E3, E2, 6K, E1, and TF (transframe). An alphavirus particle consists of a nucleocapsid core (NC) and glycoprotein layer. The NC consists of the capsid protein encapsulating the RNA genome with T=4 quasi-icosahedral symmetry and is comprised of 12 pentamers or 30 hexamers around the icosahedral five-fold (15) or two-fold (12) vertices, respectively. The NC is further encapsulated by the icosahedral glycoprotein layer consisting of the E2 and E1 single-pass transmembrane glycoproteins embedded in a host-derived lipid bilayer. The E2 and E1 glycoproteins form trimers of heterodimers arranged as 80 trimeric spikes on the surface of an alphavirus particle, with 20 spikes at the icosahedral three-fold (i3) and 60 spikes at the quasi-three-fold (93) axes^{6,17}.

The E2 glycoprotein forms the outermost layer of the viral particle and occludes most of the underlying E1 protein^{6,19}. The E2 glycoprotein engages cell-surface receptors and attachment factors, while the E1 glycoprotein lies tangential to the viral membrane and is responsible for endosomal viral fusion^{6,16,19-22}. The E2 glycoprotein consists of three immunoglobulin-like domains (A, B, and C), of which a “cleft” formed between domains A and B is involved in receptor interactions²¹⁻³¹. In the past few years, several bona fide entry receptors were identified for alphaviruses through CRISPR/Cas9-based genetic screening approaches. Different receptors were identified for the arthritogenic (i.e., Mxra8^{23,24}) and encephalitic (i.e., VEEV: LDLRAD3^{25,26}) alphaviruses, which may contribute to differences in pathogenesis²¹. More recently, members of the low-density lipoprotein receptor (LDLR) family, very low-density lipoprotein receptor (VLDLR)^{28,29,31}, apolipoprotein E receptor 2 (ApoER2)²⁸, and LDLR³⁰ were identified as receptors for EEEV. Domain B also helps prevent premature exposure of the E1 fusion loop^{6,20,22,32,33}.

Previous studies have described neutralizing anti-alphavirus antibodies that target different epitopes on the E2 glycoprotein (i.e., domains A, B, and A/B) and can inhibit either cell attachment, receptor binding, viral fusion, virus egress, or a combination of these steps^{9,34-40}. On the other hand, several anti-E1 glycoprotein neutralizing antibodies have been described and appear to primarily block viral egress^{41,42}. Nevertheless, there are a limited number of studies describing antibodies that specifically target quaternary epitopes on alphavirus particles.

### SUMMARY

Thus, in accordance with the present disclosure, there is provided a method of detecting an Eastern Equine Encephalitis Virus (EEEV) infection in a subject comprising (a) contacting a sample from said subject with an antibody or antibody fragment having clone-paired heavy and light chain CDR sequences from Tables 3 and 4, respectively; and (b) detecting EEEV in said sample by binding of said antibody or antibody fragment to a EEEV antigen in said sample. The sample may be a body fluid or tissue, and more specifically may be blood, sputum, tears, saliva, mucous or serum, semen, cervical or vaginal secretions, amniotic fluid, placental tissues, urine, exudate, transudate, tissue scrapings or feces. Detection may comprise ELISA, RIA, lateral flow assay or Western blot. The method may further comprise performing steps (a) and (b) a second time and determining a change in EEEV antigen levels as compared to the first assay. The antibody or antibody fragment may be encoded by clone-paired variable sequences as set forth in Table 1, by light and heavy chain variable sequences having 70%, 80%, or 90% identity to clone-paired variable sequences as set forth in Table 1, or by light and heavy chain variable sequences having 95% identity to clone-paired sequences as set forth in Table 1. The antibody or antibody fragment may comprise light and heavy chain variable sequences according to clone-paired sequences from Table 2, may comprise light and heavy chain variable sequences having 70%, 80% or 90% identity to clone-paired sequences from Table 2, or may comprise light and heavy chain variable sequences having 95% identity to clone-paired sequences from Table 2. The antibody fragment may be a recombinant scFv (single chain fragment variable) antibody, Fab fragment, F(ab′)₂ fragment, or Fv fragment.

In another embodiment, there is provided a method of treating a subject infected with Eastern Equine Encephalitis Virus (EEEV) or reducing the likelihood of infection of a subject at risk of contracting EEEV, comprising delivering to said subject an antibody or antibody fragment having clone-paired heavy and light chain CDR sequences from Tables 3 and 4, respectively. The antibody or antibody fragment may be encoded by clone-paired variable sequences as set forth in Table 1, by light and heavy chain variable sequences having 70%, 80%, or 90% identity to clone-paired variable sequences as set forth in Table 1, or by light and heavy chain variable sequences having 95% identity to clone-paired sequences as set forth in Table 1. The antibody or antibody fragment may comprise light and heavy chain variable sequences according to clone-paired sequences from Table 2, may comprise light and heavy chain variable sequences having 70%, 80% or 90% identity to clone-paired sequences from Table 2, or may comprise light and heavy chain variable sequences having 95% identity to clone-paired sequences from Table 2. The antibody fragment may be a recombinant scFv (single chain fragment variable) antibody, Fab fragment, F(ab′)₂ fragment, or Fv fragment. The antibody may be an IgG, or a recombinant IgG antibody or antibody fragment comprising an Fc portion mutated to alter (eliminate or enhance) FcR interactions, to increase half-life and/or increase therapeutic efficacy, such as a LALA, N297, GASD/ALIE, YTE or LS mutation or glycan modified to alter (eliminate or enhance) FcR interactions such as enzymatic or chemical addition or removal of glycans or expression in a cell line engineered with a defined glycosylating pattern. The antibody may be a chimeric antibody or a bispecific antibody. The antibody or antibody fragment may be administered prior to infection or after infection. The subject may be a pregnant female, a sexually active female, or a female undergoing fertility treatments. Delivering may comprise antibody or antibody fragment administration, or genetic delivery with an RNA or DNA sequence or vector encoding the antibody or antibody fragment.

In yet another embodiment, there is provided a monoclonal antibody, wherein the antibody or antibody fragment is characterized by clone-paired heavy and light chain CDR sequences from Tables 3 and 4, respectively. The antibody or antibody fragment may be encoded by clone-paired variable sequences as set forth in Table 1, by light and heavy chain variable sequences having 70%, 80%, or 90% identity to clone-paired variable sequences as set forth in Table 1, or by light and heavy chain variable sequences having 95% identity to clone-paired sequences as set forth in Table 1. The antibody or antibody fragment may comprise light and heavy chain variable sequences according to clone-paired sequences from Table 2, may comprise light and heavy chain variable sequences having 70%, 80% or 90% identity to clone-paired sequences from Table 2, or may comprise light and heavy chain variable sequences having 95% identity to clone-paired sequences from Table 2. The antibody fragment may be a recombinant scFv (single chain fragment variable) antibody, Fab fragment, F(ab′)₂ fragment, or Fv fragment. The antibody may be an IgG, or a recombinant IgG antibody or antibody fragment comprising an Fc portion mutated to alter (eliminate or enhance) FcR interactions, to increase half-life and/or increase therapeutic efficacy, such as a LALA, N297, GASD/ALIE, YTE or LS mutation or glycan modified to alter (eliminate or enhance) FcR interactions such as enzymatic or chemical addition or removal of glycans or expression in a cell line engineered with a defined glycosylating pattern. The antibody may be a chimeric antibody or a bispecific antibody. The antibody or antibody fragment may further comprise a cell penetrating peptide and/or is an intrabody.

In a further embodiment, there is provided a hybridoma or engineered cell encoding an antibody or antibody fragment wherein the antibody or antibody fragment is characterized by clone-paired heavy and light chain CDR sequences from Tables 3 and 4, respectively. The encoded antibody or antibody fragment may be encoded by clone-paired variable sequences as set forth in Table 1, by light and heavy chain variable sequences having 70%, 80%, or 90% identity to clone-paired variable sequences as set forth in Table 1, or by light and heavy chain variable sequences having 95% identity to clone-paired sequences as set forth in Table 1. The encoded antibody or antibody fragment may comprise light and heavy chain variable sequences according to clone-paired sequences from Table 2, may comprise light and heavy chain variable sequences having 70%, 80% or 90% identity to clone-paired sequences from Table 2, or may comprise light and heavy chain variable sequences having 95% identity to clone-paired sequences from Table 2. The encoded antibody fragment may be a recombinant scFv (single chain fragment variable) antibody, Fab fragment, F(ab′)₂ fragment, or Fv fragment. The encoded antibody may be an IgG, or a recombinant IgG antibody or antibody fragment comprising an Fc portion mutated to alter (eliminate or enhance) FcR interactions, to increase half-life and/or increase therapeutic efficacy, such as a LALA, N297, GASD/ALIE, YTE or LS mutation or glycan modified to alter (eliminate or enhance) FcR interactions such as enzymatic or chemical addition or removal of glycans or expression in a cell line engineered with a defined glycosylating pattern. The encoded antibody may be a chimeric antibody or a bispecific antibody. The encoded antibody or antibody fragment further comprises a cell penetrating peptide and/or is an intrabody.

In still a further embodiment, there is provided a vaccine formulation comprising one or more antibodies or antibody fragments characterized by clone-paired heavy and light chain CDR sequences from Tables 3 and 4, respectively. The at least one antibody or antibody fragment may be encoded by clone-paired variable sequences as set forth in Table 1, by light and heavy chain variable sequences having 70%, 80%, or 90% identity to clone-paired variable sequences as set forth in Table 1, or by light and heavy chain variable sequences having 95% identity to clone-paired sequences as set forth in Table 1. The at least one antibody or antibody fragment may comprise light and heavy chain variable sequences according to clone-paired sequences from Table 2, may comprise light and heavy chain variable sequences having 70%, 80% or 90% identity to clone-paired sequences from Table 2, or may comprise light and heavy chain variable sequences having 95% identity to clone-paired sequences from Table 2. The at least one antibody fragment may be a recombinant scFv (single chain fragment variable) antibody, Fab fragment, F(ab′)₂ fragment, or Fv fragment. The antibody may be an IgG, or a recombinant IgG antibody or antibody fragment comprising an Fc portion mutated to alter (eliminate or enhance) FcR interactions, to increase half-life and/or increase therapeutic efficacy, such as a LALA, N297, GASD/ALIE, YTE or LS mutation or glycan modified to alter (eliminate or enhance) FcR interactions such as enzymatic or chemical addition or removal of glycans or expression in a cell line engineered with a defined glycosylating pattern. The at least one antibody may be a chimeric antibody or a bispecific antibody. The at least one antibody or antibody fragment may further comprise a cell penetrating peptide and/or is an intrabody.

Additionally, there is provided a vaccine formulation comprising one or more expression vectors encoding a first antibody or antibody fragment as described herein. The expression vector(s) may be Sindbis virus or VEE vector(s). The vaccine formulation may be formulated for delivery by needle injection, jet injection, or electroporation. The vaccine formulation further comprising one or more expression vectors encoding for a second antibody or antibody fragment, such as a distinct antibody or antibody fragment as described herein.

Still another embodiment is a method of protecting the health of a placenta and/or fetus of a pregnant subject infected with or at risk of infection with Eastern Equine Encephalitis Virus (EEEV) comprising delivering to said subject an antibody or antibody fragment having clone-paired heavy and light chain CDR sequences from Tables 3 and 4, respectively. The antibody or antibody fragment may be encoded by clone-paired variable sequences as set forth in Table 1, by light and heavy chain variable sequences having 70%, 80%, or 90% identity to clone-paired variable sequences as set forth in Table 1, or by light and heavy chain variable sequences having 95% identity to clone-paired sequences as set forth in Table 1. The antibody or antibody fragment may comprise light and heavy chain variable sequences according to clone-paired sequences from Table 2, may comprise light and heavy chain variable sequences having 70%, 80% or 90% identity to clone-paired sequences from Table 2, or may comprise light and heavy chain variable sequences having 95% identity to clone-paired sequences from Table 2. The antibody fragment may be a recombinant scFv (single chain fragment variable) antibody, Fab fragment, F(ab′)₂ fragment, or Fv fragment. The antibody may be an IgG, or a recombinant IgG antibody or antibody fragment comprising an Fc portion mutated to alter (eliminate or enhance) FcR interactions, to increase half-life and/or increase therapeutic efficacy, such as a LALA, N297, GASD/ALIE, YTE or LS mutation or glycan modified to alter (eliminate or enhance) FcR interactions such as enzymatic or chemical addition or removal of glycans or expression in a cell line engineered with a defined glycosylating pattern. The antibody may be a chimeric antibody or a bispecific antibody. The antibody or antibody fragment may be administered prior to infection or after infection. The subject may be a pregnant female, a sexually active female, or a female undergoing fertility treatments. Delivering may comprise antibody or antibody fragment administration, or genetic delivery with an RNA or DNA sequence or vector encoding the antibody or antibody fragment.

The antibody or antibody fragment may increase the size of the placenta as compared to an untreated control, and/or reduce viral load and/or pathology of the fetus as compared to an untreated control.

An additional embodiment is a method of determining the antigenic integrity, correct conformation and/or correct sequence of a Eastern Equine Encephalitis Virus (EEEV) antigen comprising (a) contacting a sample comprising said antigen with a first antibody or antibody fragment having clone-paired heavy and light chain CDR sequences from Tables 3 and 4, respectively; and (b) determining antigenic integrity, correct conformation and/or correct sequence of said antigen by detectable binding of said first antibody or antibody fragment to said antigen. The sample may comprise recombinantly produced antigen or a vaccine formulation or vaccine production batch. Detection may comprise ELISA, RIA, western blot, a biosensor using surface plasmon resonance or biolayer interferometry, or flow cytometric staining. The first antibody or antibody fragment may be encoded by clone-paired variable sequences as set forth in Table 1, by light and heavy chain variable sequences having 70%, 80%, or 90% identity to clone-paired variable sequences as set forth in Table 1, or by light and heavy chain variable sequences having 95% identity to clone-paired sequences as set forth in Table 1. The first antibody or antibody fragment may comprise light and heavy chain variable sequences according to clone-paired sequences from Table 2, may comprise light and heavy chain variable sequences having 70%, 80% or 90% identity to clone-paired sequences from Table 2, or may comprise light and heavy chain variable sequences having 95% identity to clone-paired sequences from Table 2. The first antibody fragment may be a recombinant scFv (single chain fragment variable) antibody, Fab fragment, F(ab′)₂ fragment, or Fv fragment. The method may further comprise performing steps (a) and (b) a second time to determine the antigenic stability of the antigen over time.

The method may further comprise (c) contacting a sample comprising said antigen with a second antibody or antibody fragment having clone-paired heavy and light chain CDR sequences from Tables 3 and 4, respectively; and (d) determining antigenic integrity of said antigen by detectable binding of said second antibody or antibody fragment to said antigen. The second antibody or antibody fragment may be encoded by clone-paired variable sequences as set forth in Table 1, by light and heavy chain variable sequences having 70%, 80%, or 90% identity to clone-paired variable sequences as set forth in Table 1, or by light and heavy chain variable sequences having 95% identity to clone-paired sequences as set forth in Table 1. The second antibody or antibody fragment may comprise light and heavy chain variable sequences according to clone-paired sequences from Table 2, may comprise light and heavy chain variable sequences having 70%, 80% or 90% identity to clone-paired sequences from Table 2, or may comprise light and heavy chain variable sequences having 95% identity to clone-paired sequences from Table 2. The second antibody fragment may be a recombinant scFv (single chain fragment variable) antibody, Fab fragment, F(ab′)₂ fragment, or Fv fragment. The method may further comprise performing steps (c) and (d) a second time to determine the antigenic stability of the antigen over time.

Also provided is a human monoclonal antibody or antibody fragment, or hybridoma or engineered cell producing the same, wherein said antibody binds to Eastern Equine Encephalitis Virus (EEEV) E1 protein and either (a) binds to but does not neutralize EEEV or (b) binds to EEEV E1 protein and neutralizes EEEV.

The use of the word “a” or “an” when used in conjunction with the term “comprising” in the claims and/or the specification may mean “one,” but it is also consistent with the meaning of “one or more,” “at least one,” and “one or more than one.” The word “about” means plus or minus 5% of the stated number.

It is contemplated that any method or composition described herein can be implemented with respect to any other method or composition described herein. Other objects, features and advantages of the present disclosure will become apparent from the following detailed description. It should be understood, however, that the detailed description and the specific examples, while indicating specific embodiments of the disclosure, are given by way of illustration only, since various changes and modifications within the spirit and scope of the disclosure will become apparent to those skilled in the art from this detailed description.

### BRIEF DESCRIPTION OF THE DRAWINGS

The patent or application file contains at least one drawing executed in color. Copies of this patent or patent application publication with color drawing(s) will be provided by the Office upon request and payment of the necessary fec.

The following drawings form part of the present specification and are included to further demonstrate certain aspects of the present disclosure. The disclosure may be better understood by reference to one or more of these drawings in combination with the detailed description of specific embodiments presented herein.

FIGS. 1A-E. EEEV-373 potently neutralizes and protects against wild-type EEEV. (FIG. 1A) Neutralization curves of EEEV-373 (hybridoma-derived; green squares) as bivalent IgG1 molecules against wild-type EEEV strain FL93-939 in a plaque reduction neutralization assay. The previously published irrelevant isotype mAb (rDENV-2D22; black squares) or mouse anti-EEEV ascites fluid (ATCC (+) ve; purple squares) were included as negative or positive controls, respectively³⁹. Each curve displays mAb concentration (nM) or ascites fluid dilution on the x-axis and percent relative viral infectivity on the y-axis. Data represent mean±SD of technical triplicates of one biological replicate. (FIG. 1B) Percent of EEEV strain FL93-939 residual fraction (y-axis) present at the highest concentration tested (75 nM) of mAb (EEEV-373 [2%], rDENV-2D22 [99%], or ascites fluid [0%]). (FIGS. 1C-E) EEEV (strain FL93-939; 10^{3.3} CCID₅₀) was inoculated subcutaneously (s.c.) into C57BL/6 mice. After 24 hours, EEEV-373 (green) as bivalent IgG1 molecules was administered intraperitoneally (i.p.) at 10 mg/kg (200 μg/mouse; n=10). The previously published negative control treatment group, rDENV-2D22 (black; n=10), or the mock-inoculated normal controls (grey; n=5) 42 also were included for comparative purposes. (FIG. 1C) EEEV-373 (green circles) or mock-inoculated normal control (grey circles) groups mediated 100% survival compared to the negative control mAb (rDENV-2D22; black circles) treatment group. Survival curves were compared using the log-rank test (*p<0.0001). (FIG. 1D) Percent body weight change of each mouse from the EEEV-373 (green circles), rDENV-2D22 (black circles), or mock-inoculated normal control (grey circles) groups over the course of 18 days post-inoculation. (FIG. 1E) Serum was collected 3 days post-inoculation from each mouse of the treatment groups (EEEV-373 [green circles], rDENV-2D22 [black circles], or mock-inoculated normal controls [grey circles]) for quantification of virus titer (log 10 CCID₅₀/mL; y-axis) in an infectious cell culture assay. The EEEV-373 and mock-inoculated normal control groups were compared to the rDENV-2D22 negative control treatment group using an ordinary one-way ANOVA with Dunnett's multiple comparisons test (*p<0.05).

FIGS. 2A-E. EEEV-373 binds and neutralizes EEEV in a bivalent manner. (FIG. 2A) Representative binding curves of EEEV-373 as bivalent IgG1 (left), F(ab′)₂ (middle), or monovalent Fab (right) molecules to EEEV virus-like particles (VLPs; green circles), Western equine encephalitis virus (WEEV) VLPs (black circles), recombinant EEEV E2/E1 (maroon circles), monomeric E2 (blue circles) or E1 (light purple circles) protein. Each curve displays mAb concentration (nM) on the x-axis and optical density at 450 nm on the y-axis. Data represent mean±SD of technical triplicates and are representative of two independent experiments. (FIG. 2B) Representative binding kinetic curves (black lines) of EEEV-373 as bivalent IgG1 (left), F(ab′)₂ (middle), or monovalent Fab (right) molecules to EEEV VLPs using quartz crystal microbalance biosensors (Attana). EEEV VLPs were amine-coupled to chips and quenched with ethanolamine. EEEV-373 then was incubated with the chip for 300 seconds at the labeled concentration (0.33 to 35 nM) to determine the association rates (kon) followed by incubation with buffer to determine the dissociation rates (koff). Time (in seconds) is on the x-axis and frequency (in hertz (Hz)) is on the y-axis. For analysis, a bivalent (IgG1 and F(ab′)₂) or 1:1 (Fab) binding model was used, and corresponding fitted curves are shown by the purple lines. (FIG. 2C) Representative neutralization curves of EEEV-373 (green symbols) as bivalent IgG1 (closed circles), F(ab′)₂ (open circles), or monovalent Fab (open squares) molecules against the chimeric virus, SINV/EEEV. A negative control bivalent IgG1 mAb (black circles) also was included. Each curve displays mAb concentration (nM) on the x-axis and percent relative viral infectivity on the y-axis. Data represent mean±SD of technical triplicates and are representative of two independent experiments. (FIG. 2D) Summary table of the half-maximal effective (EC₅₀) concentration values for binding of EEEV-373 as bivalent IgG1 (left column), F(ab′)₂ (middle column), or monovalent Fab (right column) molecules to EEEV VLPs or recombinant EEEV E2/E1 protein, and half-maximal inhibitory (IC₅₀) values for neutralization activity against SINV/EEEV. (FIG. 2E) Summary table of kinetic analyses (KD, kon, koff) for EEEV-373 as bivalent IgG1 (left column), F(ab′)₂ (middle column), or monovalent Fab (right column) molecules.

FIGS. 3A-F. Cryo-EM reconstruction of SINV/EEEV in complex with a potently neutralizing intact IgG. (FIG. 3A) Cryo-EM reconstruction of SINV/EEEV particles in complex with EEEV-373 IgG was determined by single particle averaging with icosahedral symmetry to 4.6 Å resolution. The black triangle represents the asymmetric unit, with the symmetry axes depicted by the red pentagon (5-fold axis), triangle (3-fold axis), and circle (2-fold axis) symbols. The quasi-three-fold (q3) spikes are represented by the blue triangles. The scale bar (in A) represents the radial distance from the center of the virus. (FIG. 3B) A close-up view of the i2 axis displays a single EEEV-373 IgG molecule diagonally spanning across the axis to connect two q3 spikes. The q3 and icosahedral three-fold (13) spikes are labeled in dark blue text accordingly. In comparison to the SINV/EEEV particle density, the density of the IgG molecule is relatively weak, and to help elucidate the complex, the two arms of the IgG molecule are indicated by the dark blue dotted lines. (FIG. 3C and FIG. 3D) Localized reconstruction, focused classification, and refinement with C1 symmetry of the two q3 spikes cross-linked by the EEEV-373 IgG molecule were performed to improve the resolution of the reconstruction. A top (FIG. 3C) or side (FIG. 3D) view of the improved reconstruction from the i2 axis perspective are illustrated. The q3 spikes, constant and variable domains of EEEV-373 are indicated accordingly. The map is colored according to the coloring of the fitted model in ChimeraX. The grey regions fall slightly outside of the fitted model, while the relative protein domains of E1 (domain I: orange, domain II: light pink, domain III: light orange), E2 (domain A: cyan, domain B: teal, domain C: aqua, β-ribbon connector: purple), EEEV-373 heavy chain (green) and light chain (yellow) are colored accordingly. (FIG. 3E and FIG. 3F) Local resolution map of the volume as shown in FIG. 3C and FIG. 3D. FIG. 3E and FIG. 3F correspond to the viewing directions in FIG. 3C and FIG. 3D, respectively. The scale bar (in Å) represents the resolution gradient.

FIGS. 4A-C. EEEV-373 epitope binding footprint on SINV/EEEV particles. (FIG. 4A) RIVEM (Radial Interpretation of Viral Electron Density Maps) representation of the EEEV-373 IgG binding epitope on the viral surface. Residues within 6 Å of the complementarity-determining regions (CDRs) of EEEV-373 IgG are colored yellow and constitute the epitope binding footprint on the viral surface. The scale bar represents the radial distance in A from the center of the virus. (FIG. 4B) Cartoon representation of one arm of the EEEV-373 IgG molecule in complex with a unit of the SINV/EEEV E2/E1 heterodimer. The different protein domains of E1 (domain I: orange, domain II: light pink, domain III: tan), E2 (domain A: cyan, domain B: teal, domain C: aqua, β-ribbon connector: light purple), EEEV-373 heavy chain (green; VH: variable domain, CH1: constant domain), and EEEV-373 light chain (yellow; VL: variable domain, CL: constant domain) are labeled accordingly. (FIG. 4C) A close-up view of the interface between EEEV-373 and the SINV/EEEV E2/E1 heterodimer. The heavy (green) or light (yellow) chain CDRs 1-3 are indicated as HCDR1, HCDR2, HCDR3 or LCDR1, LCDR2, LCDR3, respectively. The E2 protein domain A (cyan), domain B (teal), β-ribbon connector (light purple), and E1 protein domain II (light pink) are labeled accordingly.

FIGS. 5A-G. EEEV-373 binds a conformational epitope on the SINV/EEEV E2 protein. (FIG. 5A) A close-up view of the E2 protein β-ribbon connector (cyan) and the E1 protein domain II (orange) interface, further highlighted by the dotted purple circle. The residues comprising the binding footprint of EEEV-373 are colored red. (FIG. 5B) A side view (rotated) ˜90° of the E2/E1 interface is shown to highlight the significant interaction of the underlying E1 protein (orange) for the proper conformation and stabilization of the E2 protein β-ribbon connector (cyan). Residues involved in this interaction are indicated by the cyan or red spheres. The red spheres indicate the E2 residues that constitute the EEEV-373 epitope binding footprint. (FIG. 5C and FIG. 5D) A detailed cartoon representation view (FIG. 5C) and a close-up, side view (rotated ˜90°; FIG. 5D) of the interactions between the heavy chain complementarity-determining region loops (HCDR1, HCDR2, and HCDR3) of EEEV-373 (green) and the ‘arch 2’ cleft of the E2 protein β-ribbon connector (cyan). (FIG. 5E and FIG. 5F) Specific view of the heavy chain CDR3 loop (HCDR3) of EEEV-373 (green) inserting within the ‘arch 2’ cleft of the E2 protein β-ribbon connector (cyan) as shown by the cartoon (FIG. 5E) or sphere (FIG. 5F) representations. The E2 protein β-ribbon connector residues that form the epitope binding footprint of EEEV-373 are indicated by the red spheres. Additional residues on the E2 protein within the immediate vicinity of the footprint are indicated by the cyan spheres. (FIG. 5G) The quality of the model fit to the map in the epitope-paratope region is colored according to the Q-score. The corresponding color key is shown on the right.

FIGS. 6A-G. Potential binding modes of EEEV-373 IgG at the icosahedral 2-fold axis of SINV/EEEV particles. (FIG. 6A) Preferred binding mode orientation of EEEV-373 IgG across the icosahedral 2-fold (12) axis (q3.1-q3.3). A cartoon representation of the fitted atomic models of the E2 protein (cyan) and EEEV-373 IgG (heavy chain: green, light chain: yellow) at the i2 axis (blue circle) is shown. The E1 protein is omitted for clarity. The four q3 spikes (q3.1-q3.4) and the two i3 spikes (13.1-i3.2) are labeled in bolded text accordingly. The distance (in Å) between the Ca atoms of the last residue (Lys222; red spheres) on strain G of the constant domains (CH1) of the two Fab arms of EEEV-373 (42.5 Å) is indicated by the dotted line. (FIG. 6B) In addition to the observed binding mode, there are five alternative ways (FIGS. 6C-G) EEEV-373 could cross-link viral spikes across the i2 axis as depicted. The distance (in Å) between the Ca atoms of the last residue (Lys222; red spheres) on strain G of the constant domains (CH1) of the two Fab arms of EEEV-373 are indicated by the dotted lines (FIG. 6C [i3.1-i3.2]: 64.0 Å; E [q3.1-q3.4]: 41.0 Å; F [q3.1-13.2]: 48.9 Å; G [q3.3-q3.4]: 44.2 Å). (FIG. 6D and FIG. 6G) For the i3.1-q3.1 (D) or q3.3-q3.4 (G) binding modes, steric clashes are observed between the light chain constant domains of EEEV-373 (yellow) as indicated by the black dotted circle (FIG. 6D) or the black box (FIG. 6G). (FIG. 6E) For the q3.1-q3.4 binding mode, movement of the constant domains as indicated by the purple arrows may lead to steric clashes. (FIG. 6F) For the q3.1-i3.2 binding mode, EEEV-373 may also bind in this orientation. However, a 60° rotation of one Fab arm with respect to the other Fab arm may reduce the likelihood of binding. The distance and steric hindrance constraints observed in FIGS. 6C-G likely contribute to the observation of EEEV-373 in the preferred binding mode orientation (FIG. 6A) with binding of the two Fab arms to the q3.1 and q3.3 spikes across the i2 axis.

FIGS. 7A-F. EEEV-373 inhibits SINV/EEEV entry through receptor blockade and inhibition of early fusion events. (FIG. 7A and FIG. 7B) Competition-binding ELISA to assess the ability of bivalent EEEV-373 IgG1 molecules to block ApoER2 (FIG. 7A) or VLDLR (FIG. 7B) protein receptors from binding to EEEV VLPs. EEEV VLPs were incubated with EEEV-373 (green) or an isotype control (black) mAb at the concentrations indicated (67 to 3 nM; x-axis). ApoER2 (5 μg/mL; FIG. 7A) or VLDLR (5 μg/mL; FIG. 7B) then were incubated with the VLPs and detected using an anti-6×-His tag-HRP conjugated secondary antibody. The average optical density at 450 nm of the isotype control mAb at each concentration was calculated and used to normalize the percent binding of EEEV-373 to EEEV VLPs (y-axis). Data represent mean±SD of technical triplicates of two independent experiments. Data were analyzed at the specified concentrations using multiple unpaired t-tests (***p<0.000001, **p<0.0001, *p<0.001) (FIG. 7C) Dynamic light scattering (DLS) of EEEV-373 (green) as bivalent IgG1 (top), F(ab′)₂ (middle), or monovalent Fab (bottom) molecules at different Ab:VLP molar ratios (x-axis). The peak hydrodynamic diameter (nm) is shown on the y-axis. Data represent mean±SD of technical duplicates and are representative of two independent experiments. (FIG. 7D) Representative neutralization curves of EEEV-373 (green) as bivalent IgG1 (top), F(ab′)₂ (middle), or monovalent Fab (bottom) molecules assessed by a post-attachment assay (open circles) or focus reduction neutralization test (FRNT; closed circles). Ab concentration (nM) is on the x-axis and percent relative infectivity is shown on the y-axis. Data represent mean±SD of technical duplicates and are representative of two independent experiments. (FIG. 7E) Liposomal fusion assay of DiD-labeled SINV/EEEV particles incubated with liposomes in the presence of EEEV-373 (closed green circles), EEEV-94 (closed cyan circles; positive control), or an isotype-matched control (open black squares; negative control) as bivalent IgG1 (top left), F(ab′)₂ (top right), or monovalent Fab (bottom left) molecules. Assay controls in which no antibody (closed black circles) or liposomes (closed grey circles) were also included. Data are representative of two independent experiments. (FIG. 7F) An egress inhibition assay was performed to determine the relative RNA copies/μL present in supernatant harvested at either 1 hour (left) or 6 hours (right) after addition of EEEV-373 (green) or the irrelevant isotype control mAb, rDENV-2D22 (black), as bivalent IgG1 molecules. RNA copies/μL were determined using a standard curve with quantitative EEEV RNA (ATCC). SINV/EEEV RNA levels were compared to the SINV/EEEV only control (purple) using an ordinary one-way ANOVA with Dunnett's multiple comparisons test. Data represent mean±SD of technical quadruplicates and are representative of two independent experiments.

FIGS. 8A-B. Binding kinetic analyses of EEEV-373 to EEEV VLPs using biolayer interferometry. (FIG. 8A) Representative binding kinetic curves of EEEV-373 as bivalent IgG1 (left; blue) or F(ab′)₂ (right; orange) molecules to EEEV VLPs using biolayer interferometry (Sartorius). Bivalent molecules of EEEV-373 at 10 μg/mL were captured by protein L biosensors and incubated with EEEV VLPs at varying concentrations (0.2 to 2 nM) for 300 seconds to determine the association rates of EEEV-373 binding. Dissociation was performed by incubation of the biosensor with kinetics buffer for 1,800 seconds. Time (in seconds) is shown on the x-axis and nm shift is on the y-axis. For data analysis, reference wells containing kinetics buffer only were subtracted from each molecule type for normalization. A 1:1 binding model was then used for analysis and fitted curves are shown by the red lines. Data are representative of at least two independent experiments. In the top right, a summary table of corresponding representative kon values for bivalent EEEV-373 IgG or F(ab′)₂ molecule binding to EEEV VLPs is shown. Only the calculated kon values (1/Ms) are reported as variable dissociation of antibody directly from protein L biosensors led to an increase in nm shift during the dissociation step following normalization. (FIG. 8B) Representative raw data binding curves for a bivalent isotype negative control mAb (top), or monovalent EEEV-373 (middle) or EEEV-94 (bottom) Fab molecules to EEEV VLPs using biolayer interferometry. Bivalent or monovalent molecules at 10 or 6.7 μg/mL, respectively, were captured by protein L biosensors and monitored for binding to EEEV VLPs as described in A. Time (in seconds) is shown on the x-axis and nm shift is on the y-axis. Data are representative of two independent experiments.

FIGS. 9A-C. EEEV-373 epitope binding footprint on the SINV/EEEV E2 protein. (FIG. 9A) The atomic models of one Fab arm of EEEV-373 (heavy chain: green, light chain: yellow), E2 protein (cyan), and E1 protein (orange) were fit into the electron density to depict the binding interface between EEEV-373 and the E2 protein. The density is represented as a partially transparent surface and colored according to the respective structures. Domain B of the E2 protein, domain II of the E1 protein, and the complementarity determining region (CDR) loops of EEEV-373 (heavy chain: HCDR1, HCDR2, and HCDR3; light chain: LCDR1, LCDR2, and LCDR3) are labeled accordingly. (FIG. 9B) Residues within 6 Å of the CDR loops of EEEV-373 IgG are colored red on the E2 protein (cyan) and constitute the epitope binding footprint on the viral surface. EEEV-373 binds to domain B and the β-ribbon connector of the E2 protein. EEEV-373 is faded for clarity and the variable heavy (VH; green) and light (VL; yellow) chains are labeled accordingly. (FIG. 9C) A rotated 90° view is also shown to provide a different view of the EEEV-373 and E2 protein interface.

FIGS. 10A-C. Generation of SINV/EEEV escape mutant viruses under EEEV-373 selective pressure. (FIG. 10A) Representative neutralization curves of EEEV-373 (green circles) or an isotype negative control (black circles) mAb against SINV/EEEV using a real-time cell analysis (RTCA) assay. Each curve displays mAb concentration (μg/mL) on the x-axis and percent neutralization on the y-axis. Data represent mean±SD of technical triplicates. (FIG. 10B) Representative images of the RTCA assay, displaying cell index values on the y-axis over time (in seconds) on the x-axis. EEEV-373 (top) or an isotype negative control (bottom) mAb at 50 (left) or 10 (right) μg/mL were incubated with SINV/EEEV. Medium (bottom left) or virus (bottom right) only controls were included for comparison. Images shaded in blue indicate wells in which a reduction in neutralization potency was observed and further analyzed for sequence analyses. (FIG. 10C) Cartoon image representations of the EEEV-373 binding footprint (red spheres) and E147K (magenta spheres) escape mutant virus on the trimeric (left) or heterodimer (right) view of the E2 (cyan) and E1 (orange) proteins.

FIGS. 11A-C. Multiple sequence alignment of the E2 protein from representative alphaviruses. (FIG. 11A) An alignment of representative E2 protein sequences of EEEV (AHL83785.1), WEEV (ACT75276.1), VEEV (AAA42997.1), CHIKV (AAA53256.3), RRV (ABB53381.1), or MAYV (AZM66146.1) was generated using COBALT (SEQ ID NOs: 13-18). Full or partially conserved residues are colored in red or blue, respectively. Grey residues indicate columns with gaps. Upper- or lower-case residues indicate less or greater than 50% gaps compared to the number of sequences, respectively. E2 protein regions are labeled underneath the alignment and colored accordingly (N-linker—grey, domain A—cyan, β-ribbon connector—light purple, domain B—teal, domain C—aqua, stem—purple, transmembrane domain—black, and cytoplasmic tail—red). The ‘arch’ regions are labeled above the alignment. The residues of the EEEV E2 protein that constitute the epitope binding footprint of EEEV-373 are shown in bold letters and highlighted with a green line above the alignment. Residues within the immediate vicinity of the footprint in the β-ribbon connector (see FIGS. 5A-G) are indicated by the yellow boxes. The VLDLR receptor binding site (‘HKR’ loop) is indicated by the red box³¹. The residue corresponding to the E147K escape mutant virus is highlighted with an orange line above the alignment. The VLDLR³¹ (FIG. 11B) or EEEV-373 (FIG. 11C) binding footprints are shown by the light blue (FIG. 11B) or red (FIG. 11C) spheres, respectively, on a zoomed-in view of the E2 (cyan) and E1 (orange) proteins to visually compare VLDLR and EEEV-373 binding.

FIGS. 12A-E. Binding mode comparison of EEEV-373 IgG versus CHK-263 Fab to the SINV/EEEV or CHIKV E2/E1 heterodimer. (FIG. 12A) Ribbon representation of one Fab arm of EEEV-373 bound to the SINV/EEEV E2/E1 heterodimer protein. The proteins are colored accordingly: E2 protein-cyan, E1 protein-orange, EEEV-373 heavy chain-green, and EEEV-373 light chain-yellow. The E2 domains are labeled as A, B, or C. The E1 domains are labeled as I, II, or III. For EEEV-373 Fab, the antibody variable domains are labeled as VH or VL and the constant domains as CH1 or CL. (FIG. 12B) Ribbon representation of CHK-263 Fab bound to the CHIKV E2/E1 heterodimer protein (PDB: 7CVZ). The proteins are colored accordingly: E2 protein—pink, E1 protein—red, CHK-263 heavy chain—purple, and CHK-263 light chain—grey. The protein domains are labeled as described in FIG. 12A. (FIG. 12C) Superimposition of EEEV-373 and CHK-263 Fab molecules bound to either the SINV/EEEV or CHIKV E2/E1 heterodimer proteins, respectively. In comparison to EEEV-373, CHK-263 tilts towards and makes contacts with the E1 protein. Furthermore, CHKV-263 engages with domain B of the E2 protein via the heavy chain, whereas EEEV-373 does so with the light chain. (FIG. 12D) A close-up view of the superimposed binding interface of EEEV-373 or CHK-263 Fab molecules to their respective antigen. To determine the angular disposition of the Fab binding to their respective epitopes, the center of mass (COM) for each Fab was calculated and compared with respect to residue valine 204, at the top of domain B of the SINV/EEEV E2 protein (indicated by the dotted arrow). The CoM for EEEV-373 or CHK-263 is shown as blue or brown spheres, respectively. (FIG. 12E) To provide a clear view of the Fab binding angular disposition to valine 204, the ribbon representations for EEEV-373 and CHK-263 Fab molecules were omitted. In comparison to EEEV-373, the CHK-263 Fab molecule tilts about 17.5° towards the E1 protein.

FIGS. 13A-D. Representative micrographs and 2D class averages of the SINV/EEEV and EEEV-373 IgG complex and comparison of densities at the icosahedral 5-fold (15) and 2-fold (12) axes. (FIG. 13A and FIG. 13B) Representative micrographs of SINV/EEEV particles in complex with EEEV-373 IgG1 molecules. The white arrows show the protruding IgG from the surface of the viral particles. In these micrographs, the particles are well dispersed (FIG. 13A) or show some clustering of virus particles (FIG. 13B), despite being in complex with EEEV-373. The nominal magnification for both images is 64,000× with a defocus of −2 μm. (FIG. 13C) Representative 2D class averages of SINV/EEEV particles in complex with EEEV-373 IgG1 molecules. The projections from the viral surface clearly illustrate bound EEEV-373. (FIG. 13D) Density of bound EEEV-373 IgG1 molecules at the i5 (left) or i2 (right) axes of symmetry at two different contour levels, a=5 (top) and a=8 (bottom). The IgG density at the i5 axis with a contour of a=8 drops sharply in comparison to the i2 axis density, suggesting very low occupancy at the i5 axis.

FIGS. 14A-G. Flow chart of cryo-EM data processing steps for SINV/EEEV particles in complex with EEEV-373 IgG1 molecules. (FIG. 14A) The resolution after icosahedral reconstruction of SINV/EEEV particles in complex with EEEV-373 IgG1 molecules was 4.6 Å. The volume around the icosahedral 2-fold (12) axis is shown by the black circle. At this stage, the density of the bound IgG was poor. (FIG. 14B) Ten classes of complexes were identified following sub-particle extraction of the i2 region and 3D classification. The respective percentage of each class are indicated. The best classes (4 and 5; dashed red box) were selected and combined to move forward through the workflow. (FIG. 14C) A localized reconstruction of the i2 region was performed to improve the resolution to 4.3 Å. Although this approach improved the density of the bound IgG, atomic model fitting could not be performed. (FIG. 14D) A mask (white outline) was applied to the bound IgG and cross-linked q3.1-q3.3 spikes. A focused classification within the masked volume was then performed. (FIG. 14E) Three classes were generated, with class 1 showing the best structural features and representation of 93.6% of the sub-particles. (FIG. 14F) Following focused classification and refinement, the density of the bound IgG improved. (FIG. 14G) The Fourier Shell Correlation (FSC) curve shows an estimated global resolution of 3.8 Å based on a 0.143 FSC coefficient cutoff. All refinements were performed with independent half-sets of data.

FIG. 15. Flow cytometric analysis of minimal EEEV-373 IgG binding to Expi293F cells transfected with the EEEV structural polyprotein. Representative flow cytometry data plots showing Expi293F cells that were transiently transfected with the structural polyprotein (capsid-E3-E2-6K-E1) of EEEV (strain FL93-939). Cells were stained with EEEV-373 (green; top), a positive control mAb, rEEEV-106 (purple; middle), or a negative control mAb, rDENV-2D22 (black; bottom). Number of events is on the y-axis and the BL2-H channel, which enables detection of cells stained with the anti-human IgG-PE-conjugated secondary antibody, is on the x-axis. Two biological replicates are displayed (left and right); data represent technical triplicates.

### DESCRIPTION OF ILLUSTRATIVE EMBODIMENTS

To further the understanding of antibody-antigen interactions, the inventors describe here the structural and molecular analyses of a potently neutralizing human monoclonal antibody (mAb) designated EEEV-373 as a bivalent IgG1 molecule in complex with the chimeric virus Sindbis (SINV)/Eastern equine encephalitis virus (EEEV). The structural analyses here reveal a quaternary epitope on the E2 glycoprotein targeted by EEEV-373. Potent efficacy of EEEV-373 was confirmed in cell culture and a mouse model of EEEV infection. This mAb distinctively binds by cross-linking two q3-spikes diagonally across the i2 axis governed by steric and distance constraints. Kinetic analysis of the antibody-antigen interaction provided mechanistic insights into the strict requirement for bivalent heterodimer binding. These data expand the scope of structure-guided immunogen design for clinically relevant alphaviruses. Furthermore, this study advances the growing knowledge on the complexity of antibody-antigen interactions for therapeutic antibody development. These and other aspects of the disclosure are described in detail below.

### I. Eastern Equine Encephalitis (EEE) and EEE Virus (EEEV)

Eastern equine encephalitis (EEE), commonly called Triple E or sleeping sickness (not to be confused with African trypanosomiasis), is a disease caused by a zoonotic mosquito vectored Togavirus that is present in North, Central, and South America, and the Caribbean. EEE was first recognized in Massachusetts, United States, in 1831, when 75 horses died mysteriously of viral encephalitis. Epizootics in horses have continued to occur regularly in the United States. It can also be identified in donkeys and zebras. Due to the rarity of the disease, its occurrence can cause economic impact beyond the cost of horses and poultry. EEE is found today in the eastern part of the United States and is often associated with coastal plains. It can most commonly be found in East Coast and Gulf Coast states. In Florida, about one to two human cases are reported a year, although over 60 cases of equine encephalitis are reported. In years in which conditions are favorable for the disease, the number of equine cases is over 200. Diagnosing equine encephalitis is challenging because many of the symptoms are shared with other illnesses and patients can be asymptomatic. Confirmations may require a sample of cerebral spinal fluid or brain tissue, although CT scans and MRI scans are used to detect encephalitis. This could be an indication that the need to test for EEE is necessary. If a biopsy of the cerebral spinal fluid is taken, it is sent to a specialized laboratory for testing.

The incubation period for Eastern equine encephalitis virus (EEEV) disease ranges from 4 to 10 days. The illness can progress either systematically or encephalitically, depending on the person's age. Encephalitic disease involves swelling of the brain and can be asymptomatic, while systemic illness occurs very abruptly. Those with the systemic illness usually recover within 1-2 weeks. While the encephalitis is more common among infants, in adults and children, it usually manifests after experiencing the systemic illness. Symptoms include high fever, muscle pain, altered mental status, headache, meningeal irritation, photophobia, and seizures, which occur 3-10 days after the bite of an infected mosquito. Due to the virus's effect on the brain, patients who survive can be left with mental and physical impairments, such as personality disorders, paralysis, seizures, and intellectual impairment.

EEEV is capable of infecting a wide range of animals, including mammals, birds, reptiles, and amphibians. The virus is maintained in nature through a bird-mosquito cycle. Two mosquito species are primarily involved in this portion of the cycle; they are Culiseta melanura and Culiseta morsitans. These mosquitoes feed on the blood of birds. The frequency of the virus found in nature increases throughout the summer as more birds and more mosquitoes become infected.

Transmission of EEEV to mammals (including humans) occurs via other mosquito species, which feed on the blood of both birds and mammals. These other mosquitoes are referred to as “bridge vectors” because they carry the virus from the avian hosts to other types of hosts, particularly mammals. The bridge vectors include Aedes taeniorhynchus, Aedes vexans, Coquillettidia perturbans, Ochlerotatus canadensis, and Ochlerotatus sollicitans. Ochlerotatus canadensis also frequently bites turtles.

Humans, horses, and most other infected mammals do not circulate enough viruses in their blood to infect additional mosquitoes. Some cases of EEE have been contracted through laboratory exposures or from exposure of the eyes, lungs, or skin wounds to brain or spinal cord matter from infected animals.

The disease can be prevented in horses with the use of vaccinations, which are usually given with vaccinations for other diseases, most commonly western equine encephalitis virus, Venezuelan equine encephalitis virus, and tetanus. Most vaccinations for EEE consist of the killed virus. For humans, no vaccine for EEE is available; prevention involves reducing the risk of exposure. Using insect repellent, wearing protective clothing, and reducing the amount of standing water is the best means for prevention.

No cure for EEE has been found. Treatment consists of corticosteroids, anticonvulsants, and supportive measures (treating symptoms) such as intravenous fluids, tracheal intubation, and antipyretics. About 4% of humans known to be infected develop symptoms, with a total of about six cases per year in the US. A third of these cases die, and many survivors suffer permanent brain damage.

Several states in the Northeast U.S. have had increased virus activity since 2004. Between 2004 and 2006, at least ten human cases of EEE were reported in Massachusetts. In 2006, about 500,000 acres (2,000 km²) in southeastern Massachusetts were treated with mosquito adulticides to reduce the risk of humans contracting EEE. Several human cases were reported in New Hampshire, as well. On 19 Jul. 2012, the virus was identified in a mosquito of the species Coquillettidia perturbans in Nickerson State Park on Cape Cod, Massachusetts. On 28 Jul. 2012, the virus was found in mosquitos in Pittsfield, Massachusetts. As of September 2019, a notable uptick in cases erupted in New England and Michigan, prompting some health departments to declare an outbreak. As of 31 Oct. 2019, five people died in Michigan, three people died in Connecticut, one person died in Rhode Island, one person died in Alabama, one person died in Indiana, and three people died in Massachusetts. The virus was also found in goats, in turkeys, in deer, and in horses. As of Sep. 9, 2020, there were 5 confirmed human cases between Massachusetts and Wisconsin. As of Oct. 9, 2020, one person died in Michigan and one person died in Wisconsin.

In October 2007, a citizen of Livingston, West Lothian, Scotland became the first European victim of this disease. The man had visited New Hampshire during the summer of 2007, on a fishing vacation, and was diagnosed as having EEE on 13 Sep. 2007. He fell ill with the disease on 31 Aug. 2007, just one day after flying home, and later fell into a coma. He later awoke from the coma with severe brain injuries.

From its natural reservoir in birds, EEEV is known to infect reptiles and amphibians as well as both humans and other mammals, including horses. After inoculation by the vector, the virus travels via lymphatics to lymph nodes, and replicates in macrophages and neutrophils, resulting in lymphopenia, leukopenia, and fever. Subsequent replication occurs in other organs, leading to viremia.

Symptoms in horses occur 1-3 weeks after infection, and begin with a fever that may reach as high as 106° F. (41° C.). The fever usually lasts for 24-48 hours. Nervous signs appear during the fever that include sensitivity to sound, periods of excitement, and restlessness. Brain lesions appear, causing drowsiness, drooping cars, circling, aimless wandering, head pressing, inability to swallow, and abnormal gait. Paralysis follows, causing the horse to have difficulty raising its head. The horse usually suffers complete paralysis and death 2-4 days after symptoms appear. Mortality rates among horses with the eastern strain range from 70 to 90%.

### II. Monoclonal Antibodies and Production Thereof

An “isolated antibody” is one that has been separated and/or recovered from a component of its natural environment. Contaminant components of its natural environment are materials that would interfere with diagnostic or therapeutic uses for the antibody, and may include enzymes, hormones, and other proteinaceous or non-proteinaceous solutes. In particular embodiments, the antibody is purified: (1) to greater than 95% by weight of antibody as determined by the Lowry method, and most particularly more than 99% by weight; (2) to a degree sufficient to obtain at least 15 residues of N-terminal or internal amino acid sequence by use of a spinning cup sequenator; or (3) to homogeneity by SDS-PAGE under reducing or non-reducing conditions using Coomassie blue or silver stain. Isolated antibody includes the antibody in situ within recombinant cells since at least one component of the antibody's natural environment will not be present. Ordinarily, however, isolated antibody will be prepared by at least one purification step.

The basic four-chain antibody unit is a heterotetrameric glycoprotein composed of two identical light (L) chains and two identical heavy (H) chains. An IgM antibody consists of 5 basic heterotetramer units along with an additional polypeptide called J chain, and therefore contain 10 antigen binding sites, while secreted IgA antibodies can polymerize to form polyvalent assemblages comprising 2-5 of the basic 4-chain units along with J chain. In the case of IgGs, the 4-chain unit is generally about 150,000 daltons. Each L chain is linked to an H chain by one covalent disulfide bond, while the two H chains are linked to each other by one or more disulfide bonds depending on the H chain isotype. Each H and L chain also has regularly spaced intrachain disulfide bridges. Each H chain has at the N-terminus, a variable region (V_{H}) followed by three constant domains (C_{H}) for each of the alpha and gamma chains and four C_{H} domains for mu and isotypes. Each L chain has at the N-terminus, a variable region (V_{L}) followed by a constant domain (C_{L}) at its other end. The V_{L} is aligned with the V_{H} and the C_{L} is aligned with the first constant domain of the heavy chain (C_{H1}). Particular amino acid residues are believed to form an interface between the light chain and heavy chain variable regions. The pairing of a V_{H} and V_{L} together forms a single antigen-binding site. For the structure and properties of the different classes of antibodies, see, e.g., Basic and Clinical Immunology, 8th edition, Daniel P. Stites, Abba I. Terr and Tristram G. Parslow (eds.), Appleton & Lange, Norwalk, Conn., 1994, page 71, and Chapter 6.

The L chain from any vertebrate species can be assigned to one of two clearly distinct types, called kappa and lambda based on the amino acid sequences of their constant domains (C_{L}). Depending on the amino acid sequence of the constant domain of their heavy chains (C_{H}), immunoglobulins can be assigned to different classes or isotypes. There are five classes of immunoglobulins: IgA, IgD, IgE, IgG, and IgM, having heavy chains designated alpha, delta, epsilon, gamma and mu, respectively. They gamma and alpha classes are further divided into subclasses on the basis of relatively minor differences in C_{H} sequence and function, humans express the following subclasses: IgG1, IgG2, IgG3, IgG4, IgA1, and IgA2.

The term “variable” refers to the fact that certain segments of the V domains differ extensively in sequence among antibodies. The V domain mediates antigen binding and defines specificity of a particular antibody for its particular antigen. However, the variability is not evenly distributed across the 110-amino acid span of the variable regions. Instead, the V regions consist of relatively invariant stretches called framework regions (FRs) of 15-30 amino acids separated by shorter regions of extreme variability called “hypervariable regions” that are each 9-12 amino acids long. The variable regions of native heavy and light chains each comprise four FRs, largely adopting a beta-sheet configuration, connected by three hypervariable regions, which form loops connecting, and in some cases forming part of, the beta-sheet structure. The hypervariable regions in each chain are held together in close proximity by the FRs and, with the hypervariable regions from the other chain, contribute to the formation of the antigen-binding site of antibodies (see Kabat et al., Sequences of Proteins of Immunological Interest, 5th Ed. Public Health Service, National Institutes of Health, Bethesda, Md. (1991)). The constant domains are not involved directly in binding an antibody to an antigen, but exhibit various effector functions, such as participation of the antibody in antibody dependent cellular cytotoxicity (ADCC), antibody-dependent cellular phagocytosis (ADCP), antibody-dependent neutrophil phagocytosis (ADNP), and antibody-dependent complement deposition (ADCD).

The term “hypervariable region” when used herein refers to the amino acid residues of an antibody that are responsible for antigen binding. The hypervariable region generally comprises amino acid residues from a “complementarity determining region” or “CDR” (e.g., around about residues 24-34 (L1), 50-56 (L2) and 89-97 (L3) in the V_{L}, and around about 31-35 (H1), 50-65 (H2) and 95-102 (H3) in the V_{H} when numbered in accordance with the Kabat numbering system; Kabat et al., Sequences of Proteins of Immunological Interest, 5th Ed. Public Health Service, National Institutes of Health, Bethesda, Md. (1991)); and/or those residues from a “hypervariable loop” (e.g., residues 24-34 (L1), 50-56 (L2) and 89-97 (L3) in the V_{L}, and 26-32 (H1), 52-56 (H2) and 95-101 (H3) in the V_{H} when numbered in accordance with the Chothia numbering system; Chothia and Lesk, J. Mol. Biol. 196:901-917 (1987)); and/or those residues from a “hypervariable loop”/CDR (e.g., residues 27-38 (L1), 56-65 (L2) and 105-120 (L3) in the V_{L}, and 27-38 (H1), 56-65 (H2) and 105-120 (H3) in the V_{H} when numbered in accordance with the IMGT numbering system; Lefranc, M. P. et al. Nucl. Acids Res. 27:209-212 (1999), Ruiz, M. et al. Nucl. Acids Res. 28:219-221 (2000)). Optionally the antibody has symmetrical insertions at one or more of the following points 28, 36 (L1), 63, 74-75 (L2) and 123 (L3) in the V_{L}, and 28, 36 (H1), 63, 74-75 (H2) and 123 (H3) in the V_{sub}H when numbered in accordance with AHo; Honneger, A. and Plunkthun, A. J. Mol. Biol. 309:657-670 (2001)).

By “germline nucleic acid residue” is meant the nucleic acid residue that naturally occurs in a germline gene encoding a constant or variable region. “Germline gene” is the DNA found in a germ cell (i.e., a cell destined to become an egg or in the sperm). A “germline mutation” refers to a heritable change in a particular DNA that has occurred in a germ cell or the zygote at the single-cell stage, and when transmitted to offspring, such a mutation is incorporated in every cell of the body. A germline mutation is in contrast to a somatic mutation which is acquired in a single body cell. In some cases, nucleotides in a germline DNA sequence encoding for a variable region are mutated (i.e., a somatic mutation) and replaced with a different nucleotide.

The term “monoclonal antibody” as used herein refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigenic site. Furthermore, in contrast to polyclonal antibody preparations that include different antibodies directed against different determinants (epitopes), each monoclonal antibody is directed against a single determinant on the antigen. In addition to their specificity, the monoclonal antibodies are advantageous in that they may be synthesized uncontaminated by other antibodies. The modifier “monoclonal” is not to be construed as requiring production of the antibody by any particular method. For example, the monoclonal antibodies useful in the present disclosure may be prepared by the hybridoma methodology first described by Kohler et al., Nature, 256:495 (1975), or may be made using recombinant DNA methods in bacterial, eukaryotic animal or plant cells (see, e.g., U.S. Pat. No. 4,816,567) after single cell sorting of an antigen specific B cell, an antigen specific plasmablast responding to an infection or immunization, or capture of linked heavy and light chains from single cells in a bulk sorted antigen specific collection. The “monoclonal antibodies” may also be isolated from phage antibody libraries using the techniques described in Clackson et al., Nature, 352:624-628 (1991) and Marks et al., J. Mol. Biol., 222:581-597 (1991), for example.

### A. General Methods

It will be understood that monoclonal antibodies binding to EEEV will have several applications. These include the production of diagnostic kits for use in detecting and diagnosing EEEV infection, as well as for treating the same. In these contexts, one may link such antibodies to diagnostic or therapeutic agents, use them as capture agents or competitors in competitive assays, or use them individually without additional agents being attached thereto. The antibodies may be mutated or modified, as discussed further below. Methods for preparing and characterizing antibodies are well known in the art (see, e.g., Antibodies: A Laboratory Manual, Cold Spring Harbor Laboratory, 1988; U.S. Pat. No. 4,196,265).

The methods for generating monoclonal antibodies (MAbs) generally begin along the same lines as those for preparing polyclonal antibodies. The first step for both these methods is immunization of an appropriate host or identification of subjects who are immune due to prior natural infection or vaccination with a licensed or experimental vaccine. As is well known in the art, a given composition for immunization may vary in its immunogenicity. It is often necessary therefore to boost the host immune system, as may be achieved by coupling a peptide or polypeptide immunogen to a carrier. Exemplary and preferred carriers are keyhole limpet hemocyanin (KLH) and bovine serum albumin (BSA). Other albumins such as ovalbumin, mouse serum albumin or rabbit serum albumin can also be used as carriers. Means for conjugating a polypeptide to a carrier protein are well known in the art and include glutaraldehyde, m-maleimidobencoyl-N-hydroxysuccinimide ester, carbodiimyde and bis-biazotized benzidine. As also is well known in the art, the immunogenicity of a particular immunogen composition can be enhanced by the use of non-specific stimulators of the immune response, known as adjuvants. Exemplary and preferred adjuvants in animals include complete Freund's adjuvant (a non-specific stimulator of the immune response containing killed Mycobacterium tuberculosis), incomplete Freund's adjuvants and aluminum hydroxide adjuvant and in humans include alum, CpG, MFP59 and combinations of immunostimulatory molecules (“Adjuvant Systems”, such as AS01 or AS03). Additional experimental forms of inoculation to induce EEEV-specific B cells is possible, including nanoparticle vaccines, or gene-encoded antigens delivered as DNA or RNA genes in a physical delivery system (such as lipid nanoparticle or on a gold biolistic bead), and delivered with needle, gene gun, transcutaneous electroporation device. The antigen gene also can be carried as encoded by a replication competent or defective viral vector such as adenovirus, adeno-associated virus, poxvirus, herpesvirus, or alphavirus replicon, or alternatively a virus like particle.

In the case of human antibodies against natural pathogens, a suitable approach is to identify subjects that have been exposed to the pathogens, such as those who have been diagnosed as having contracted the disease, or those who have been vaccinated to generate protective immunity against the pathogen or to test the safety or efficacy of an experimental vaccine. Circulating anti-pathogen antibodies can be detected, and antibody encoding or producing B cells from the antibody-positive subject may then be obtained.

The amount of immunogen composition used in the production of polyclonal antibodies varies upon the nature of the immunogen as well as the animal used for immunization. A variety of routes can be used to administer the immunogen (subcutaneous, intramuscular, intradermal, intravenous and intraperitoneal). The production of polyclonal antibodies may be monitored by sampling blood of the immunized animal at various points following immunization. A second, booster injection, also may be given. The process of boosting and titering is repeated until a suitable titer is achieved. When a desired level of immunogenicity is obtained, the immunized animal can be bled and the serum isolated and stored, and/or the animal can be used to generate MAbs.

Following immunization, somatic cells with the potential for producing antibodies, specifically B lymphocytes (B cells), are selected for use in the MAb generating protocol. These cells may be obtained from biopsied spleens, lymph nodes, tonsils or adenoids, bone marrow aspirates or biopsies, tissue biopsies from mucosal organs like lung or GI tract, or from circulating blood. The antibody-producing B lymphocytes from the immunized animal or immune human are then fused with cells of an immortal myeloma cell, generally one of the same species as the animal that was immunized or human or human/mouse chimeric cells. Myeloma cell lines suited for use in hybridoma-producing fusion procedures preferably are non-antibody-producing, have high fusion efficiency, and enzyme deficiencies that render then incapable of growing in certain selective media which support the growth of only the desired fused cells (hybridomas). Any one of a number of myeloma cells may be used, as are known to those of skill in the art (Goding, pp. 65-66, 1986; Campbell, pp. 75-83, 1984). HMMA2.5 cells or MFP-2 cells are particularly useful examples of such cells.

Methods for generating hybrids of antibody-producing spleen or lymph node cells and myeloma cells usually comprise mixing somatic cells with myeloma cells in a 2:1 proportion, though the proportion may vary from about 20:1 to about 1:1, respectively, in the presence of an agent or agents (chemical or electrical) that promote the fusion of cell membranes. In some cases, transformation of human B cells with Epstein Barr virus (EBV) as an initial step increases the size of the B cells, enhancing fusion with the relatively large-sized myeloma cells. Transformation efficiency by EBV is enhanced by using CpG and a Chk2 inhibitor drug in the transforming medium. Alternatively, human B cells can be activated by co-culture with transfected cell lines expressing CD40 Ligand (CD154) in medium containing additional soluble factors, such as IL-21 and human B cell Activating Factor (BAFF), a Type II member of the TNF superfamily. Fusion methods using Sendai virus have been described by Kohler and Milstein (1975; 1976), and those using polyethylene glycol (PEG), such as 37% (v/v) PEG, by Gefter et al. (1977). The use of electrically induced fusion methods also is appropriate (Goding, pp. 71-74, 1986) and there are processes for better efficiency (Yu et al., 2008). Fusion procedures usually produce viable hybrids at low frequencies, about 1×10⁻⁶ to 1×10⁻⁸, but with optimized procedures one can achieve fusion efficiencies close to 1 in 200 (Yu et al., 2008). However, relatively low efficiency of fusion does not pose a problem, as the viable, fused hybrids are differentiated from the parental, infused cells (particularly the infused myeloma cells that would normally continue to divide indefinitely) by culturing in a selective medium. The selective medium is generally one that contains an agent that blocks the de novo synthesis of nucleotides in the tissue culture medium. Exemplary and preferred agents are aminopterin, methotrexate, and azaserine. Aminopterin and methotrexate block de novo synthesis of both purines and pyrimidines, whereas azaserine blocks only purine synthesis. Where aminopterin or methotrexate is used, the medium is supplemented with hypoxanthine and thymidine as a source of nucleotides (HAT medium). Where azaserine is used, the medium is supplemented with hypoxanthine. Ouabain is added if the B cell source is an EBV-transformed human B cell line, in order to eliminate EBV-transformed lines that have not fused to the myeloma.

The preferred selection medium is HAT or HAT with ouabain. Only cells capable of operating nucleotide salvage pathways are able to survive in HAT medium. The myeloma cells are defective in key enzymes of the salvage pathway, e.g., hypoxanthine phosphoribosyl transferase (HPRT), and they cannot survive. The B cells can operate this pathway, but they have a limited life span in culture and generally die within about two weeks. Therefore, the only cells that can survive in the selective media are those hybrids formed from myeloma and B cells. When the source of B cells used for fusion is a line of EBV-transformed B cells, as here, ouabain may also be used for drug selection of hybrids as EBV-transformed B cells are susceptible to drug killing, whereas the myeloma partner used is chosen to be ouabain resistant.

Culturing provides a population of hybridomas from which specific hybridomas are selected. Typically, selection of hybridomas is performed by culturing the cells by single-clone dilution in microtiter plates, followed by testing the individual clonal supernatants (after about two to three weeks) for the desired reactivity. The assay should be sensitive, simple and rapid, such as radioimmunoassays, enzyme immunoassays, cytotoxicity assays, plaque assays dot immunobinding assays, and the like. The selected hybridomas are then serially diluted or single-cell sorted by flow cytometric sorting and cloned into individual antibody-producing cell lines, which clones can then be propagated indefinitely to provide mAbs. The cell lines may be exploited for MAb production in two basic ways. A sample of the hybridoma can be injected (often into the peritoneal cavity) into an animal (e.g., a mouse). Optionally, the animals are primed with a hydrocarbon, especially oils such as pristane (tetramethylpentadecane) prior to injection. When human hybridomas are used in this way, it is optimal to inject immunocompromised mice, such as SCID mice, to prevent tumor rejection. The injected animal develops tumors secreting the specific monoclonal antibody produced by the fused cell hybrid. The body fluids of the animal, such as serum or ascites fluid, can then be tapped to provide MAbs in high concentration. The individual cell lines could also be cultured in vitro, where the MAbs are naturally secreted into the culture medium from which they can be readily obtained in high concentrations. Alternatively, human hybridoma cells lines can be used in vitro to produce immunoglobulins in cell supernatant. The cell lines can be adapted for growth in serum-free medium to optimize the ability to recover human monoclonal immunoglobulins of high purity.

MAbs produced by either means may be further purified, if desired, using filtration, centrifugation and various chromatographic methods such as FPLC or affinity chromatography. Fragments of the monoclonal antibodies of the disclosure can be obtained from the purified monoclonal antibodies by methods which include digestion with enzymes, such as pepsin or papain, and/or by cleavage of disulfide bonds by chemical reduction. Alternatively, monoclonal antibody fragments encompassed by the present disclosure can be synthesized using an automated peptide synthesizer.

It also is contemplated that a molecular cloning approach may be used to generate monoclonal antibodies. Single B cells labelled with the antigen of interest can be sorted physically using paramagnetic bead selection or flow cytometric sorting, then RNA can be isolated from the single cells and antibody genes amplified by RT-PCR. Alternatively, antigen-specific bulk sorted populations of cells can be segregated into microvesicles and the matched heavy and light chain variable genes recovered from single cells using physical linkage of heavy and light chain amplicons, or common barcoding of heavy and light chain genes from a vesicle. Matched heavy and light chain genes form single cells also can be obtained from populations of antigen specific B cells by treating cells with cell-penetrating nanoparticles bearing RT-PCR primers and barcodes for marking transcripts with one barcode per cell. The antibody variable genes also can be isolated by RNA extraction of a hybridoma line and the antibody genes obtained by RT-PCR and cloned into an immunoglobulin expression vector. Alternatively, combinatorial immunoglobulin phagemid libraries are prepared from RNA isolated from the cell lines and phagemids expressing appropriate antibodies are selected by panning using viral antigens. The advantages of this approach over conventional hybridoma techniques are that approximately 10⁴ times as many antibodies can be produced and screened in a single round, and that new specificities are generated by H and L chain combination which further increases the chance of finding appropriate antibodies.

Other U.S. patents, each incorporated herein by reference, that teach the production of antibodies useful in the present disclosure include U.S. Pat. No. 5,565,332, which describes the production of chimeric antibodies using a combinatorial approach; U.S. Pat. No. 4,816,567 which describes recombinant immunoglobulin preparations; and U.S. Pat. No. 4,867,973 which describes antibody-therapeutic agent conjugates.

### B. Antibodies of the Present Disclosure

Antibodies according to the present disclosure may be defined, in the first instance, by their binding specificity. Those of skill in the art, by assessing the binding specificity/affinity of a given antibody using techniques well known to those of skill in the art, can determine whether such antibodies fall within the scope of the instant claims. For example, the epitope to which a given antibody bind may consist of a single contiguous sequence of 3 or more (e.g., 3, 4, 5, 6, 7, 8, 9, 10, 11, 12, 13, 14, 15, 16, 17, 18, 19, 20) amino acids located within the antigen molecule (e.g., a linear epitope in a domain). Alternatively, the epitope may consist of a plurality of non-contiguous amino acids (or amino acid sequences) located within the antigen molecule (e.g., a conformational epitope).

Various techniques known to persons of ordinary skill in the art can be used to determine whether an antibody “interacts with one or more amino acids” within a polypeptide or protein. Exemplary techniques include, for example, routine cross-blocking assays, such as that described in Antibodies, Harlow and Lane (Cold Spring Harbor Press, Cold Spring Harbor, N.Y.). Cross-blocking can be measured in various binding assays such as ELISA, biolayer interferometry, or surface plasmon resonance. Other methods include alanine scanning mutational analysis, peptide blot analysis (Reineke (2004) Methods Mol. Biol. 248:443-63), peptide cleavage analysis, high-resolution electron microscopy techniques using single particle reconstruction, cryoEM, or tomography, crystallographic studies and NMR analysis. In addition, methods such as epitope excision, epitope extraction and chemical modification of antigens can be employed (Tomer (2000) Prot. Sci. 9:487-496). Another method that can be used to identify the amino acids within a polypeptide with which an antibody interacts is hydrogen/deuterium exchange detected by mass spectrometry. In general terms, the hydrogen/deuterium exchange method involves deuterium-labeling the protein of interest, followed by binding the antibody to the deuterium-labeled protein. Next, the protein/antibody complex is transferred to water and exchangeable protons within amino acids that are protected by the antibody complex undergo deuterium-to-hydrogen back-exchange at a slower rate than exchangeable protons within amino acids that are not part of the interface. As a result, amino acids that form part of the protein/antibody interface may retain deuterium and therefore exhibit relatively higher mass compared to amino acids not included in the interface. After dissociation of the antibody, the target protein is subjected to protease cleavage and mass spectrometry analysis, thereby revealing the deuterium-labeled residues which correspond to the specific amino acids with which the antibody interacts. Sec, e.g., Ehring (1999) Analytical Biochemistry 267:252-259; Engen and Smith (2001) Anal. Chem. 73: 256A-265A. When the antibody neutralizes EEEV, antibody escape mutant variant organisms can be isolated by propagating EEEV in vitro or in animal models in the presence of high concentrations of the antibody. Sequence analysis of the EEEV gene encoding the antigen targeted by the antibody reveals the mutation(s) conferring antibody escape, indicating residues in the epitope or that affect the structure of the epitope allosterically.

The term “epitope” refers to a site on an antigen to which B and/or T cells respond. B-cell epitopes can be formed both from contiguous amino acids or non-contiguous amino acids juxtaposed by tertiary folding of a protein. Epitopes formed from contiguous amino acids are typically retained on exposure to denaturing solvents, whereas epitopes formed by tertiary folding are typically lost on treatment with denaturing solvents. An epitope typically includes at least 3, and more usually, at least 5 or 8-10 amino acids in a unique spatial conformation.

Modification-Assisted Profiling (MAP), also known as Antigen Structure-based Antibody Profiling (ASAP) is a method that categorizes large numbers of monoclonal antibodies (mAbs) directed against the same antigen according to the similarities of the binding profile of each antibody to chemically or enzymatically modified antigen surfaces (see US2004/0101920, herein specifically incorporated by reference in its entirety). Each category may reflect a unique epitope either distinctly different from or partially overlapping with epitope represented by another category. This technology allows rapid filtering of genetically identical antibodies, such that characterization can be focused on genetically distinct antibodies. When applied to hybridoma screening, MAP may facilitate identification of rare hybridoma clones that produce mAbs having the desired characteristics. MAP may be used to sort the antibodies of the disclosure into groups of antibodies binding different epitopes.

The present disclosure includes antibodies that may bind to the same epitope, or a portion of the epitope. Likewise, the present disclosure also includes antibodies that compete for binding to a target or a fragment thereof with any of the specific exemplary antibodies described herein.

One can easily determine whether an antibody binds to the same epitope as, or competes for binding with, a reference antibody by using routine methods known in the art. For example, to determine if a test antibody binds to the same epitope as a reference, the reference antibody is allowed to bind to target under saturating conditions. Next, the ability of a test antibody to bind to the target molecule is assessed. If the test antibody is able to bind to the target molecule following saturation binding with the reference antibody, it can be concluded that the test antibody binds to a different epitope than the reference antibody. On the other hand, if the test antibody is not able to bind to the target molecule following saturation binding with the reference antibody, then the test antibody may bind to the same epitope as the epitope bound by the reference antibody.

To determine if an antibody competes for binding with a reference anti-EEEV antibody, the above-described binding methodology is performed in two orientations: In a first orientation, the reference antibody is allowed to bind to the EEEV antigen under saturating conditions followed by assessment of binding of the test antibody to the EEEV molecule. In a second orientation, the test antibody is allowed to bind to the EEEV antigen molecule under saturating conditions followed by assessment of binding of the reference antibody to the EEEV molecule. If, in both orientations, only the first (saturating) antibody is capable of binding to the EEEV, then it is concluded that the test antibody and the reference antibody compete for binding to the EEEV. As will be appreciated by a person of ordinary skill in the art, an antibody that competes for binding with a reference antibody may not necessarily bind to the identical epitope as the reference antibody but may sterically block binding of the reference antibody by binding an overlapping or adjacent epitope.

Two antibodies bind to the same or overlapping epitope if each competitively inhibits (blocks) binding of the other to the antigen. That is, a 1-, 5-, 10-, 20- or 100-fold excess of one antibody inhibits binding of the other by at least 50% but preferably 75%, 90% or even 99% as measured in a competitive binding assay (see, e.g., Junghans et al., Cancer Res. 1990 50:1495-1502). Alternatively, two antibodies have the same epitope if essentially all amino acid mutations in the antigen that reduce or eliminate binding of one antibody reduce or eliminate binding of the other. Two antibodies have overlapping epitopes if some amino acid mutations that reduce or eliminate binding of one antibody reduce or eliminate binding of the other.

Additional routine experimentation (e.g., peptide mutation and binding analyses) can then be carried out to confirm whether the observed lack of binding of the test antibody is in fact due to binding to the same epitope as the reference antibody or if steric blocking (or another phenomenon) is responsible for the lack of observed binding. Experiments of this sort can be performed using ELISA, RIA, surface plasmon resonance, flow cytometry or any other quantitative or qualitative antibody-binding assay available in the art. Structural studies with EM or crystallography also can demonstrate whether or not two antibodies that compete for binding recognize the same epitope.

In another aspect, there are provided monoclonal antibodies having clone-paired CDRs from the heavy and light chains as illustrated in Tables 3 and 4, respectively. Such antibodies may be produced by the clones discussed below in the Examples section using methods described herein.

In another aspect, the antibodies may be defined by their variable sequence, which include additional “framework” regions. These are provided in Tables 1 and 2 that encode or represent full variable regions. Furthermore, the antibodies sequences may vary from these sequences, optionally using methods discussed in greater detail below. For example, nucleic acid sequences may vary from those set out above in that (a) the variable regions may be segregated away from the constant domains of the light and heavy chains, (b) the nucleic acids may vary from those set out above while not affecting the residues encoded thereby, (c) the nucleic acids may vary from those set out above by a given percentage, e.g., 70%, 75%, 80%, 85%, 90%, 91%, 92%, 93%, 94%, 95%, 96%, 97%, 98% or 99% homology, (d) the nucleic acids may vary from those set out above by virtue of the ability to hybridize under high stringency conditions, as exemplified by low salt and/or high temperature conditions, such as provided by about 0.02 M to about 0.15 M NaCl at temperatures of about 50° C. to about 70° C., (c) the amino acids may vary from those set out above by a given percentage, e.g., 80%, 85%, 90%, 91%, 92%, 93%, 94%, 95%, 96%, 97%, 98% or 99% homology, or (f) the amino acids may vary from those set out above by permitting conservative substitutions (discussed below). Each of the foregoing applies to the nucleic acid sequences set forth as Table 1 and the amino acid sequences of Table 2.

When comparing polynucleotide and polypeptide sequences, two sequences are said to be “identical” if the sequence of nucleotides or amino acids in the two sequences is the same when aligned for maximum correspondence, as described below. Comparisons between two sequences are typically performed by comparing the sequences over a comparison window to identify and compare local regions of sequence similarity. A “comparison window” as used herein, refers to a segment of at least about 20 contiguous positions, usually 30 to about 75, 40 to about 50, in which a sequence may be compared to a reference sequence of the same number of contiguous positions after the two sequences are optimally aligned.

Optimal alignment of sequences for comparison may be conducted using the Megalign program in the Lasergene suite of bioinformatics software (DNASTAR, Inc., Madison, Wis.), using default parameters. This program embodies several alignment schemes described in the following references: Dayhoff, M. O. (1978) A model of evolutionary change in proteins—Matrices for detecting distant relationships. In Dayhoff, M. O. (ed.) Atlas of Protein Sequence and Structure, National Biomedical Research Foundation, Washington D.C. Vol. 5, Suppl. 3, pp. 345-358; Hein J. (1990) Unified Approach to Alignment and Phylogeny pp. 626-645 Methods in Enzymology vol. 183, Academic Press, Inc., San Diego, Calif.; Higgins, D. G. and Sharp, P. M. (1989) CABIOS 5:151-153; Myers, E. W. and Muller W. (1988) CABIOS 4:11-17; Robinson, E. D. (1971) Comb. Theor 11:105; Santou, N. Nes, M. (1987) Mol. Biol. Evol. 4:406-425; Sneath, P. H. A. and Sokal, R. R. (1973) Numerical Taxonomy—the Principles and Practice of Numerical Taxonomy, Freeman Press, San Francisco, Calif.; Wilbur, W. J. and Lipman, D. J. (1983) Proc. Natl. Acad., Sci. USA 80:726-730.

Alternatively, optimal alignment of sequences for comparison may be conducted by the local identity algorithm of Smith and Waterman (1981) Add. APL. Math 2:482, by the identity alignment algorithm of Needleman and Wunsch (1970) J. Mol. Biol. 48:443, by the search for similarity methods of Pearson and Lipman (1988) Proc. Natl. Acad. Sci. USA 85:2444, by computerized implementations of these algorithms (GAP, BESTFIT, BLAST, FASTA, and TFASTA in the Wisconsin Genetics Software Package, Genetics Computer Group (GCG), 575 Science Dr., Madison, Wis.), or by inspection.

One particular example of algorithms that are suitable for determining percent sequence identity and sequence similarity are the BLAST and BLAST 2.0 algorithms, which are described in Altschul et al. (1977) Nucl. Acids Res. 25:3389-3402 and Altschul et al. (1990) J. Mol. Biol. 215:403-410, respectively. BLAST and BLAST 2.0 can be used, for example, with the parameters described herein, to determine percent sequence identity for the polynucleotides and polypeptides of the disclosure. Software for performing BLAST analyses is publicly available through the National Center for Biotechnology Information. The rearranged nature of an antibody sequence and the variable length of each gene requires multiple rounds of BLAST searches for a single antibody sequence. Also, manual assembly of different genes is difficult and error prone. The sequence analysis tool IgBLAST (world-wide-web at ncbi.nlm.nih.gov/igblast/) identifies matches to the germline V, D and J genes, details at rearrangement junctions, the delineation of Ig V domain framework regions and complementarity determining regions. IgBLAST can analyze nucleotide or protein sequences and can process sequences in batches and allows searches against the germline gene databases and other sequence databases simultaneously to minimize the chance of missing possibly the best matching germline V gene.

In one illustrative example, cumulative scores can be calculated using, for nucleotide sequences, the parameters M (reward score for a pair of matching residues; always >0) and N (penalty score for mismatching residues; always <0). Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T and X determine the sensitivity and speed of the alignment. The BLASTN program (for nucleotide sequences) uses as defaults a wordlength (W) of 11, and expectation (E) of 10, and the BLOSUM62 scoring matrix (see Henikoff and Henikoff (1989) Proc. Natl. Acad. Sci. USA 89:10915) alignments, (B) of 50, expectation (E) of 10, M=5, N=−4 and a comparison of both strands.

For amino acid sequences, a scoring matrix can be used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T and X determine the sensitivity and speed of the alignment.

In one approach, the “percentage of sequence identity” is determined by comparing two optimally aligned sequences over a window of comparison of at least 20 positions, wherein the portion of the polynucleotide or polypeptide sequence in the comparison window may comprise additions or deletions (i.e., gaps) of 20 percent or less, usually 5 to 15 percent, or 10 to 12 percent, as compared to the reference sequences (which does not comprise additions or deletions) for optimal alignment of the two sequences. The percentage is calculated by determining the number of positions at which the identical nucleic acid bases or amino acid residues occur in both sequences to yield the number of matched positions, dividing the number of matched positions by the total number of positions in the reference sequence (i.e., the window size) and multiplying the results by 100 to yield the percentage of sequence identity.

Yet another way of defining an antibody is as a “derivative” of any of the below-described antibodies and their antigen-binding fragments. The term “derivative” refers to an antibody or antigen-binding fragment thereof that immunospecifically binds to an antigen but which comprises, one, two, three, four, five or more amino acid substitutions, additions, deletions or modifications relative to a “parental” (or wild-type) molecule. Such amino acid substitutions or additions may introduce naturally occurring (i.e., DNA-encoded) or non-naturally occurring amino acid residues. The term “derivative” encompasses, for example, as variants having altered CH1, hinge, C_{H2}, C_{H3} or C_{H4} regions, so as to form, for example, antibodies, etc., having variant Fc regions that exhibit enhanced or impaired effector or binding characteristics. The term “derivative” additionally encompasses non-amino acid modifications, for example, amino acids that may be glycosylated (e.g., have altered mannose, 2-N-acetylglucosamine, galactose, fucose, glucose, sialic acid, 5-N-acetylneuraminic acid, 5-glycolneuraminic acid, etc. content), acetylated, pegylated, phosphorylated, amidated, derivatized by known protecting/blocking groups, proteolytic cleavage, linked to a cellular ligand or other protein, etc. In some embodiments, the altered carbohydrate modifications modulate one or more of the following: solubilization of the antibody, facilitation of subcellular transport and secretion of the antibody, promotion of antibody assembly, conformational integrity, and antibody-mediated effector function. In a specific embodiment, the altered carbohydrate modifications enhance antibody mediated effector function relative to the antibody lacking the carbohydrate modification. Carbohydrate modifications that lead to altered antibody mediated effector function are well known in the art (for example, see Shields, R. L. et al. (2002), J. Biol. Chem. 277 (30): 26733-26740; Davies J. et al. (2001), Biotechnology & Bioengineering 74 (4): 288-294). Methods of altering carbohydrate contents are known to those skilled in the art, see, e.g., Wallick, S. C. et al. (1988), J. Exp. Med. 168 (3): 1099-1109; Tao, M. H. et al. (1989), J. Immunol. 143 (8): 2595-2601; Routledge, E. G. et al. (1995), Transplantation 60 (8): 847-53; Elliott, S. et al. (2003), Nature Biotechnol. 21:414-21; Shields, R. L. et al. (2002), J. Biol. Chem. 277 (30): 26733-26740).

A derivative antibody or antibody fragment can be generated with an engineered sequence or glycosylation state to confer preferred levels of activity in antibody dependent cellular cytotoxicity (ADCC), antibody-dependent cellular phagocytosis (ADCP), antibody-dependent neutrophil phagocytosis (ADNP), or antibody-dependent complement deposition (ADCD) functions as measured by bead-based or cell-based assays or in vivo studies in animal models.

A derivative antibody or antibody fragment may be modified by chemical modifications using techniques known to those of skill in the art, including, but not limited to, specific chemical cleavage, acetylation, formulation, metabolic synthesis of tunicamycin, etc. In one embodiment, an antibody derivative will possess a similar or identical function as the parental antibody. In another embodiment, an antibody derivative will exhibit an altered activity relative to the parental antibody. For example, a derivative antibody (or fragment thereof) can bind to its epitope more tightly or be more resistant to proteolysis than the parental antibody.

### C. Engineering of Antibody Sequences

In various embodiments, one may choose to engineer sequences of the identified antibodies for a variety of reasons, such as improved expression, improved cross-reactivity or diminished off-target binding. Modified antibodies may be made by any technique known to those of skill in the art, including expression through standard molecular biological techniques, or the chemical synthesis of polypeptides. Methods for recombinant expression are addressed elsewhere in this document. The following is a general discussion of relevant goals techniques for antibody engineering.

Hybridomas may be cultured, then cells lysed, and total RNA extracted. Random hexamers may be used with RT to generate cDNA copies of RNA, and then PCR performed using a multiplex mixture of PCR primers expected to amplify all human variable gene sequences. PCR product can be cloned into pGEM-T Easy vector, then sequenced by automated DNA sequencing using standard vector primers. Assay of binding and neutralization may be performed using antibodies collected from hybridoma supernatants and purified by FPLC, using Protein G columns.

Recombinant full-length IgG antibodies can be generated by subcloning heavy and light chain Fv DNAs from the cloning vector into an IgG plasmid vector, transfected into 293 (e.g., Freestyle) cells or CHO cells, and antibodies can be collected and purified from the 293 or CHO cell supernatant. Other appropriate host cells systems include bacteria, such as E. coli, insect cells (S2, Sf9, Sf29, High Five), plant cells (e.g., tobacco, with or without engineering for human-like glycans), algae, or in a variety of non-human transgenic contexts, such as mice, rats, goats or cows.

Expression of nucleic acids encoding antibodies, both for the purpose of subsequent antibody purification, and for immunization of a host, is also contemplated. Antibody coding sequences can be RNA, such as native RNA or modified RNA. Modified RNA contemplates certain chemical modifications that confer increased stability and low immunogenicity to mRNAs, thereby facilitating expression of therapeutically important proteins. For instance, N1-methyl-pseudouridine (N1 mΨ) outperforms several other nucleoside modifications and their combinations in terms of translation capacity. In addition to turning off the immune/eIF2α phosphorylation-dependent inhibition of translation, incorporated N1mΨ nucleotides dramatically alter the dynamics of the translation process by increasing ribosome pausing and density on the mRNA. Increased ribosome loading of modified mRNAs renders them more permissive for initiation by favoring either ribosome recycling on the same mRNA or de novo ribosome recruitment. Such modifications could be used to enhance antibody expression in vivo following inoculation with RNA. The RNA, whether native or modified, may be delivered as naked RNA or in a delivery vehicle, such as a lipid nanoparticle.

Alternatively, DNA encoding the antibody may be employed for the same purposes. The DNA is included in an expression cassette comprising a promoter active in the host cell for which it is designed. The expression cassette is advantageously included in a replicable vector, such as a conventional plasmid or minivector. Vectors include viral vectors, such as poxviruses, adenoviruses, herpesviruses, adeno-associated viruses, and lentiviruses are contemplated. Replicons encoding antibody genes such as alphavirus replicons based on VEE virus or Sindbis virus are also contemplated. Delivery of such vectors can be performed by needle through intramuscular, subcutaneous, or intradermal routes, or by transcutaneous electroporation when in vivo expression is desired.

The rapid availability of antibody produced in the same host cell and cell culture process as the final cGMP manufacturing process has the potential to reduce the duration of process development programs. Lonza has developed a generic method using pooled transfectants grown in CDACF medium, for the rapid production of small quantities (up to 50 g) of antibodies in CHO cells. Although slightly slower than a true transient system, the advantages include a higher product concentration and use of the same host and process as the production cell line. Example of growth and productivity of GS-CHO pools, expressing a model antibody, in a disposable bioreactor: in a disposable bag bioreactor culture (5 L working volume) operated in fed-batch mode, a harvest antibody concentration of 2 g/L was achieved within 9 weeks of transfection.

Antibody molecules will comprise fragments (such as F(ab′), F(ab′)₂) that are produced, for example, by the proteolytic cleavage of the mAbs, or single-chain immunoglobulins producible, for example, via recombinant means. F(ab′) antibody derivatives are monovalent, while F(ab′)₂ antibody derivatives are bivalent. In one embodiment, such fragments can be combined with one another, or with other antibody fragments or receptor ligands to form “chimeric” binding molecules. Significantly, such chimeric molecules may contain substituents capable of binding to different epitopes of the same molecule.

In related embodiments, the antibody is a derivative of the disclosed antibodies, e.g., an antibody comprising the CDR sequences identical to those in the disclosed antibodies (e.g., a chimeric, or CDR-grafted antibody). Alternatively, one may wish to make modifications, such as introducing conservative changes into an antibody molecule. In making such changes, the hydropathic index of amino acids may be considered. The importance of the hydropathic amino acid index in conferring interactive biologic function on a protein is generally understood in the art (Kyte and Doolittle, 1982). It is accepted that the relative hydropathic character of the amino acid contributes to the secondary structure of the resultant protein, which in turn defines the interaction of the protein with other molecules, for example, enzymes, substrates, receptors, DNA, antibodies, antigens, and the like.

It also is understood in the art that the substitution of like amino acids can be made effectively on the basis of hydrophilicity. U.S. Pat. No. 4,554,101 (incorporated herein by reference) states that the greatest local average hydrophilicity of a protein, as governed by the hydrophilicity of its adjacent amino acids, correlates with a biological property of the protein. As detailed in U.S. Pat. No. 4,554,101, the following hydrophilicity values have been assigned to amino acid residues: basic amino acids: arginine (+3.0), lysine (+3.0), and histidine (−0.5); acidic amino acids: aspartate (+3.0±1), glutamate (+3.0±1), asparagine (+0.2), and glutamine (+0.2); hydrophilic, nonionic amino acids: serine (+0.3), asparagine (+0.2), glutamine (+0.2), and threonine (−0.4), sulfur containing amino acids: cysteine (−1.0) and methionine (−1.3); hydrophobic, nonaromatic amino acids: valine (−1.5), leucine (−1.8), isoleucine (−1.8), proline (−0.5±1), alanine (−0.5), and glycine (0); hydrophobic, aromatic amino acids: tryptophan (−3.4), phenylalanine (−2.5), and tyrosine (−2.3).

It is understood that an amino acid can be substituted for another having a similar hydrophilicity and produce a biologically or immunologically modified protein. In such changes, the substitution of amino acids whose hydrophilicity values are within ±2 is preferred, those that are within ±1 are particularly preferred, and those within ±0.5 are even more particularly preferred. As outlined above, amino acid substitutions generally are based on the relative similarity of the amino acid side-chain substituents, for example, their hydrophobicity, hydrophilicity, charge, size, and the like. Exemplary substitutions that take into consideration the various foregoing characteristics are well known to those of skill in the art and include: arginine and lysine; glutamate and aspartate; serine and threonine; glutamine and asparagine; and valine, leucine and isoleucine.

The present disclosure also contemplates isotype modification. By modifying the Fc region to have a different isotype, different functionalities can be achieved. For example, changing to IgG₁ can increase antibody dependent cell cytotoxicity, switching to class A can improve tissue distribution, and switching to class M can improve valency.

Alternatively or additionally, it may be useful to combine amino acid modifications with one or more further amino acid modifications that alter C1q binding and/or the complement dependent cytotoxicity (CDC) function of the Fc region of an IL-23p19 binding molecule. The binding polypeptide of particular interest may be one that binds to C1q and displays complement dependent cytotoxicity. Polypeptides with pre-existing C1q binding activity, optionally further having the ability to mediate CDC may be modified such that one or both of these activities are enhanced. Amino acid modifications that alter C1q and/or modify its complement dependent cytotoxicity function are described, for example, in WO/0042072, which is hereby incorporated by reference.

One can design an Fc region of an antibody with altered effector function, e.g., by modifying C1q binding and/or FcγR binding and thereby changing CDC activity and/or ADCC activity. “Effector functions” are responsible for activating or diminishing a biological activity (e.g., in a subject). Examples of effector functions include, but are not limited to: C1q binding; complement dependent cytotoxicity (CDC); Fc receptor binding; antibody-dependent cell-mediated cytotoxicity (ADCC); phagocytosis; down regulation of cell surface receptors (e.g., B cell receptor; BCR), etc. Such effector functions may require the Fc region to be combined with a binding domain (e.g., an antibody variable domain) and can be assessed using various assays (e.g., Fc binding assays, ADCC assays, CDC assays, etc.).

For example, one can generate a variant Fc region of an antibody with improved C1q binding and improved FcγRIII binding (e.g., having both improved ADCC activity and improved CDC activity). Alternatively, if it is desired that effector function be reduced or ablated, a variant Fc region can be engineered with reduced CDC activity and/or reduced ADCC activity. In other embodiments, only one of these activities may be increased, and, optionally, also the other activity reduced (e.g., to generate an Fc region variant with improved ADCC activity, but reduced CDC activity and vice versa).

FcRn binding. Fc mutations can also be introduced and engineered to alter their interaction with the neonatal Fc receptor (FcRn) and improve their pharmacokinetic properties. A collection of human Fc variants with improved binding to the FcRn have been described (Shields et al., (2001). High resolution mapping of the binding site on human IgG1 for FcγRI, FcγRII, FcγRIII, and FcRn and design of IgG1 variants with improved binding to the FcγR, (J. Biol. Chem. 276:6591-6604). A number of methods are known that can result in increased half-life (Kuo and Aveson, (2011)), including amino acid modifications may be generated through techniques including alanine scanning mutagenesis, random mutagenesis and screening to assess the binding to the neonatal Fc receptor (FcRn) and/or the in vivo behavior. Computational strategies followed by mutagenesis may also be used to select one of amino acid mutations to mutate.

The present disclosure therefore provides a variant of an antigen binding protein with optimized binding to FcRn. In a particular embodiment, the said variant of an antigen binding protein comprises at least one amino acid modification in the Fc region of said antigen binding protein, wherein said modification is selected from the group consisting of 226, 227, 228, 230, 231, 233, 234, 239, 241, 243, 246, 250, 252, 256, 259, 264, 265, 267, 269, 270, 276, 284, 285, 288, 289, 290, 291, 292, 294, 297, 298, 299, 301, 302, 303, 305, 307, 308, 309, 311, 315, 317, 320, 322, 325, 327, 330, 332, 334, 335, 338, 340, 342, 343, 345, 347, 350, 352, 354, 355, 356, 359, 360, 361, 362, 369, 370, 371, 375, 378, 380, 382, 384, 385, 386, 387, 389, 390, 392, 393, 394, 395, 396, 397, 398, 399, 400, 401 403, 404, 408, 411, 412, 414, 415, 416, 418, 419, 420, 421, 422, 424, 426, 428, 433, 434, 438, 439, 440, 443, 444, 445, 446 and 447 of the Fc region as compared to said parent polypeptide, wherein the numbering of the amino acids in the Fc region is that of the EU index in Kabat. In a further aspect of the disclosure the modifications are M252Y/S254T/T256E.

Additionally, various publications describe methods for obtaining physiologically active molecules whose half-lives are modified, see for example Kontermann (2009) either by introducing an FcRn-binding polypeptide into the molecules or by fusing the molecules with antibodies whose FcRn-binding affinities are preserved but affinities for other Fc receptors have been greatly reduced or fusing with FcRn binding domains of antibodies.

Derivatized antibodies may be used to alter the half-lives (e.g., serum half-lives) of parental antibodies in a mammal, particularly a human. Such alterations may result in a half-life of greater than 15 days, preferably greater than 20 days, greater than 25 days, greater than 30 days, greater than 35 days, greater than 40 days, greater than 45 days, greater than 2 months, greater than 3 months, greater than 4 months, or greater than 5 months. The increased half-lives of the antibodies of the present disclosure or fragments thereof in a mammal, preferably a human, results in a higher serum titer of said antibodies or antibody fragments in the mammal, and thus reduces the frequency of the administration of said antibodies or antibody fragments and/or reduces the concentration of said antibodies or antibody fragments to be administered. Antibodies or fragments thereof having increased in vivo half-lives can be generated by techniques known to those of skill in the art. For example, antibodies or fragments thereof with increased in vivo half-lives can be generated by modifying (e.g., substituting, deleting or adding) amino acid residues identified as involved in the interaction between the Fc domain and the FcRn receptor.

Beltramello et al. (2010) previously reported the modification of neutralizing mAbs, due to their tendency to enhance dengue virus infection, by generating in which leucine residues at positions 1.3 and 1.2 of C_{H2} domain (according to the IMGT unique numbering for C-domain) were substituted with alanine residues. This modification, also known as “LALA” mutation, abolishes antibody binding to FcγRI, FcγRII and FcγRIIIa, as described by Hessell et al. (2007). The variant and unmodified recombinant mAbs were compared for their capacity to neutralize and enhance infection by the four dengue virus serotypes. LALA variants retained the same neutralizing activity as unmodified mAb but were completely devoid of enhancing activity. LALA mutations of this nature are therefore contemplated in the context of the presently disclosed antibodies.

Altered Glycosylation. A particular embodiment of the present disclosure is an isolated monoclonal antibody, or antigen binding fragment thereof, containing a substantially homogeneous glycan without sialic acid, galactose, or fucose. The monoclonal antibody comprises a heavy chain variable region and a light chain variable region, both of which may be attached to heavy chain or light chain constant regions respectively. The aforementioned substantially homogeneous glycan may be covalently attached to the heavy chain constant region.

Another embodiment of the present disclosure comprises a mAb with a novel Fc glycosylation pattern. The isolated monoclonal antibody, or antigen binding fragment thereof, is present in a substantially homogenous composition represented by the GNGN or G1/G2 glycoform. Fc glycosylation plays a significant role in anti-viral and anti-cancer properties of therapeutic mAbs. The disclosure is in line with a recent study that shows increased anti-lentivirus cell-mediated viral inhibition of a fucose free anti-HIV mAb in vitro. This embodiment of the present disclosure with homogenous glycans lacking a core fucose, showed increased protection against specific viruses by a factor greater than two-fold. Elimination of core fucose dramatically improves the ADCC activity of mAbs mediated by natural killer (NK) cells but appears to have the opposite effect on the ADCC activity of polymorphonuclear cells (PMNs).

The isolated monoclonal antibody, or antigen binding fragment thereof, comprising a substantially homogenous composition represented by the GNGN or G1/G2 glycoform exhibits increased binding affinity for Fc gamma RI and Fc gamma RIII compared to the same antibody without the substantially homogeneous GNGN glycoform and with G0, GIF, G2F, GNF, GNGNF or GNGNFX containing glycoforms. In one embodiment of the present disclosure, the antibody dissociates from Fc gamma RI with a Kd of 1×10⁻⁸ M or less and from Fc gamma RIII with a Kd of 1×10⁻⁷ M or less.

Glycosylation of an Fc region is typically either N-linked or O-linked. N-linked refers to the attachment of the carbohydrate moiety to the side chain of an asparagine residue. O-linked glycosylation refers to the attachment of one of the sugars N-acetylgalactosamine, galactose, or xylose to a hydroxyamino acid, most commonly serine or threonine, although 5-hydroxyproline or 5-hydroxylysine may also be used. The recognition sequences for enzymatic attachment of the carbohydrate moiety to the asparagine side chain peptide sequences are asparagine-X-serine and asparagine-X-threonine, where X is any amino acid except proline. Thus, the presence of either of these peptide sequences in a polypeptide creates a potential glycosylation site.

The glycosylation pattern may be altered, for example, by deleting one or more glycosylation site(s) found in the polypeptide, and/or adding one or more glycosylation site(s) that are not present in the polypeptide. Addition of glycosylation sites to the Fc region of an antibody is conveniently accomplished by altering the amino acid sequence such that it contains one or more of the above-described tripeptide sequences (for N-linked glycosylation sites). An exemplary glycosylation variant has an amino acid substitution of residue Asn 297 of the heavy chain. The alteration may also be made by the addition of, or substitution by, one or more serine or threonine residues to the sequence of the original polypeptide (for O-linked glycosylation sites). Additionally, a change of Asn 297 to Ala can remove one of the glycosylation sites.

In certain embodiments, the antibody is expressed in cells that express beta (1,4)-N-acetylglucosaminyltransferase III (GnT III), such that GnT III adds GlcNAc to the IL-23p19 antibody. Methods for producing antibodies in such a fashion are provided in WO/9954342, WO/03011878, patent publication 20030003097 Å1, and Umana et al., Nature Biotechnology, 17:176-180, February 1999. Cell lines can be altered to enhance or reduce or eliminate certain post-translational modifications, such as glycosylation, using genome editing technology such as Clustered Regularly Interspaced Short Palindromic Repeats (CRISPR). For example, CRISPR technology can be used to eliminate genes encoding glycosylating enzymes in 293 or CHO cells used to express recombinant monoclonal antibodies.

Elimination of monoclonal antibody protein sequence liabilities. It is possible to engineer the antibody variable gene sequences obtained from human B cells to enhance their manufacturability and safety. Potential protein sequence liabilities can be identified by searching for sequence motifs associated with sites containing:
1) Unpaired Cys residues,
2) N-linked glycosylation,
3) Asn deamidation,
4) Asp isomerization,
5) SYE truncation,
6) Met oxidation,
7) Trp oxidation,
8) N-terminal glutamate,
9) Integrin binding,
10) CD11c/CD18 binding, or
11) Fragmentation
   Such motifs can be eliminated by altering the synthetic gene for the cDNA encoding recombinant antibodies.

Protein engineering efforts in the field of development of therapeutic antibodies clearly reveal that certain sequences or residues are associated with solubility differences (Fernandez-Escamilla et al., Nature Biotech., 22 (10), 1302-1306, 2004; Chennamsetty et al., PNAS, 106 (29), 11937-11942, 2009; Voynov et al., Biocon. Chem., 21 (2), 385-392, 2010) Evidence from solubility-altering mutations in the literature indicate that some hydrophilic residues such as aspartic acid, glutamic acid, and serine contribute significantly more favorably to protein solubility than other hydrophilic residues, such as asparagine, glutamine, threonine, lysine, and arginine.

Stability. Antibodies can be engineered for enhanced biophysical properties. One can use elevated temperature to unfold antibodies to determine relative stability, using average apparent melting temperatures. Differential Scanning calorimetry (DSC) measures the heat capacity, Cₚ, of a molecule (the heat required to warm it, per degree) as a function of temperature. One can use DSC to study the thermal stability of antibodies. DSC data for mAbs is particularly interesting because it sometimes resolves the unfolding of individual domains within the mAb structure, producing up to three peaks in the thermogram (from unfolding of the Fab, C_{H2}, and C_{H3} domains). Typically unfolding of the Fab domain produces the strongest peak. The DSC profiles and relative stability of the Fc portion show characteristic differences for the human IgG₁, IgG₂, IgG₃, and IgG₄ subclasses (Garber and Demarest, Biochem. Biophys. Res. Commun. 355, 751-757, 2007). One also can determine average apparent melting temperature using circular dichroism (CD), performed with a CD spectrometer. Far-UV CD spectra will be measured for antibodies in the range of 200 to 260 nm at increments of 0.5 nm. The final spectra can be determined as averages of 20 accumulations. Residue ellipticity values can be calculated after background subtraction. Thermal unfolding of antibodies (0.1 mg/mL) can be monitored at 235 nm from 25-95° C. and a heating rate of 1° C./min. One can use dynamic light scattering (DLS) to assess for propensity for aggregation. DLS is used to characterize size of various particles including proteins. If the system is not disperse in size, the mean effective diameter of the particles can be determined. This measurement depends on the size of the particle core, the size of surface structures, and particle concentration. Since DLS essentially measures fluctuations in scattered light intensity due to particles, the diffusion coefficient of the particles can be determined. DLS software in commercial DLA instruments displays the particle population at different diameters. Stability studies can be done conveniently using DLS. DLS measurements of a sample can show whether the particles aggregate over time or with temperature variation by determining whether the hydrodynamic radius of the particle increases. If particles aggregate, one can see a larger population of particles with a larger radius. Stability depending on temperature can be analyzed by controlling the temperature in situ. Capillary electrophoresis (CE) techniques include proven methodologies for determining features of antibody stability. One can use an iCE approach to resolve antibody protein charge variants due to deamidation, C-terminal lysines, sialylation, oxidation, glycosylation, and any other change to the protein that can result in a change in pI of the protein. Each of the expressed antibody proteins can be evaluated by high throughput, free solution isoelectric focusing (IEF) in a capillary column (cIEF), using a Protein Simple Maurice instrument. Whole-column UV absorption detection can be performed every 30 seconds for real time monitoring of molecules focusing at the isoelectric points (pIs). This approach combines the high resolution of traditional gel IEF with the advantages of quantitation and automation found in column-based separations while eliminating the need for a mobilization step. The technique yields reproducible, quantitative analysis of identity, purity, and heterogeneity profiles for the expressed antibodies. The results identify charge heterogeneity and molecular sizing on the antibodies, with both absorbance and native fluorescence detection modes and with sensitivity of detection down to 0.7 μg/mL.

Solubility. One can determine the intrinsic solubility score of antibody sequences. The intrinsic solubility scores can be calculated using CamSol Intrinsic (Sormanni et al., J Mol Biol 427, 478-490, 2015). The amino acid sequences for residues 95-102 (Kabat numbering) in HCDR3 of each antibody fragment such as a scFv can be evaluated via the online program to calculate the solubility scores. One also can determine solubility using laboratory techniques. Various techniques exist, including addition of lyophilized protein to a solution until the solution becomes saturated and the solubility limit is reached, or concentration by ultrafiltration in a microconcentrator with a suitable molecular weight cut-off. The most straightforward method is induction of amorphous precipitation, which measures protein solubility using a method involving protein precipitation using ammonium sulfate (Trevino et al., J Mol Biol, 366:449-460, 2007). Ammonium sulfate precipitation gives quick and accurate information on relative solubility values. Ammonium sulfate precipitation produces precipitated solutions with well-defined aqueous and solid phases and requires relatively small amounts of protein. Solubility measurements performed using induction of amorphous precipitation by ammonium sulfate also can be done easily at different pH values. Protein solubility is highly pH dependent, and pH is considered the most important extrinsic factor that affects solubility.

Autoreactivity. Generally, it is thought that autoreactive clones should be eliminated during ontogeny by negative selection, however it has become clear that many human naturally occurring antibodies with autoreactive properties persist in adult mature repertoires, and the autoreactivity may enhance the antiviral function of many antibodies to pathogens. It has been noted that HCDR3 loops in antibodies during early B cell development are often rich in positive charge and exhibit autoreactive patterns (Wardemann et al., Science 301, 1374-1377, 2003). One can test a given antibody for autoreactivity by assessing the level of binding to human origin cells in microscopy (using adherent HeLa or HEp-2 epithelial cells) and flow cytometric cell surface staining (using suspension Jurkat T cells and 293S human embryonic kidney cells). Autoreactivity also can be surveyed using assessment of binding to tissues in tissue arrays.

Preferred residues (“Human Likeness”). B cell repertoire deep sequencing of human B cells from blood donors is being performed on a wide scale in many recent studies. Sequence information about a significant portion of the human antibody repertoire facilitates statistical assessment of antibody sequence features common in healthy humans. With knowledge about the antibody sequence features in a human recombined antibody variable gene reference database, the position specific degree of “Human Likeness” (HL) of an antibody sequence can be estimated. HL has been shown to be useful for the development of antibodies in clinical use, like therapeutic antibodies or antibodies as vaccines. The goal is to increase the human likeness of antibodies to reduce potential adverse effects and anti-antibody immune responses that will lead to significantly decreased efficacy of the antibody drug or can induce serious health implications. One can assess antibody characteristics of the combined antibody repertoire of three healthy human blood donors of about 400 million sequences in total and created a novel “relative Human Likeness” (rHL) score that focuses on the hypervariable region of the antibody. The rHL score allows one to easily distinguish between human (positive score) and non-human sequences (negative score). Antibodies can be engineered to eliminate residues that are not common in human repertoires.

### D. Single Chain Antibodies

A single chain variable fragment (scFv) is a fusion of the variable regions of the heavy and light chains of immunoglobulins, linked together with a short (usually serine, glycine) linker. This chimeric molecule retains the specificity of the original immunoglobulin, despite removal of the constant regions and the introduction of a linker peptide. This modification usually leaves the specificity unaltered. These molecules were created historically to facilitate phage display where it is highly convenient to express the antigen binding domain as a single peptide. Alternatively, scFv can be created directly from subcloned heavy and light chains derived from a hybridoma or B cell. Single chain variable fragments lack the constant Fc region found in complete antibody molecules, and thus, the common binding sites (e.g., protein A/G) used to purify antibodies. These fragments can often be purified/immobilized using Protein L since Protein L interacts with the variable region of kappa light chains.

Flexible linkers generally are comprised of helix- and turn-promoting amino acid residues such as alanine, serine and glycine. However, other residues can function as well. Tang et al. (1996) used phage display as a means of rapidly selecting tailored linkers for single-chain antibodies (scFvs) from protein linker libraries. A random linker library was constructed in which the genes for the heavy and light chain variable domains were linked by a segment encoding an 18-amino acid polypeptide of variable composition. The scFv repertoire (approx. 5×10⁶ different members) was displayed on filamentous phage and subjected to affinity selection with hapten. The population of selected variants exhibited significant increases in binding activity but retained considerable sequence diversity. Screening 1054 individual variants subsequently yielded a catalytically active scFv that was produced efficiently in soluble form. Sequence analysis revealed a conserved proline in the linker two residues after the V_{H} C terminus and an abundance of arginines and prolines at other positions as the only common features of the selected tethers.

The recombinant antibodies of the present disclosure may also involve sequences or moieties that permit dimerization or multimerization of the receptors. Such sequences include those derived from IgA, which permit formation of multimers in conjunction with the J-chain. Another multimerization domain is the Gal4 dimerization domain. In other embodiments, the chains may be modified with agents such as biotin/avidin, which permit the combination of two antibodies.

In a separate embodiment, a single-chain antibody can be created by joining receptor light and heavy chains using a non-peptide linker or chemical unit. Generally, the light and heavy chains will be produced in distinct cells, purified, and subsequently linked together in an appropriate fashion (i.e., the N-terminus of the heavy chain being attached to the C-terminus of the light chain via an appropriate chemical bridge).

Cross-linking reagents are used to form molecular bridges that tie functional groups of two different molecules, e.g., a stabilizing and coagulating agent. However, it is contemplated that dimers or multimers of the same analog or heteromeric complexes comprised of different analogs can be created. To link two different compounds in a step-wise manner, hetero-bifunctional cross-linkers can be used that eliminate unwanted homopolymer formation.

An exemplary hetero-bifunctional cross-linker contains two reactive groups: one reacting with primary amine group (e.g., N-hydroxy succinimide) and the other reacting with a thiol group (e.g., pyridyl disulfide, maleimides, halogens, etc.). Through the primary amine reactive group, the cross-linker may react with the lysine residue(s) of one protein (e.g., the selected antibody or fragment) and through the thiol reactive group, the cross-linker, already tied up to the first protein, reacts with the cysteine residue (free sulfhydryl group) of the other protein (e.g., the selective agent).

It is preferred that a cross-linker having reasonable stability in blood will be employed. Numerous types of disulfide-bond containing linkers are known that can be successfully employed to conjugate targeting and therapeutic/preventative agents. Linkers that contain a disulfide bond that is sterically hindered may prove to give greater stability in vivo, preventing release of the targeting peptide prior to reaching the site of action. These linkers are thus one group of linking agents.

Another cross-linking reagent is SMPT, which is a bifunctional cross-linker containing a disulfide bond that is “sterically hindered” by an adjacent benzene ring and methyl groups. It is believed that steric hindrance of the disulfide bond serves a function of protecting the bond from attack by thiolate anions such as glutathione which can be present in tissues and blood, and thereby help in preventing decoupling of the conjugate prior to the delivery of the attached agent to the target site.

The SMPT cross-linking reagent, as with many other known cross-linking reagents, lends the ability to cross-link functional groups such as the SH of cysteine or primary amines (e.g., the epsilon amino group of lysine). Another possible type of cross-linker includes the hetero-bifunctional photoreactive phenylazides containing a cleavable disulfide bond such as sulfosuccinimidyl-2-(p-azido salicylamido) ethyl-1,3′-dithiopropionate. The N-hydroxy-succinimidyl group reacts with primary amino groups and the phenylazide (upon photolysis) reacts non-selectively with any amino acid residue.

In addition to hindered cross-linkers, non-hindered linkers also can be employed in accordance herewith. Other useful cross-linkers, not considered to contain or generate a protected disulfide, include SATA, SPDP and 2-iminothiolane (Wawrzynczak & Thorpe, 1987). The use of such cross-linkers is well understood in the art. Another embodiment involves the use of flexible linkers.

U.S. Pat. No. 4,680,338 describes bifunctional linkers useful for producing conjugates of ligands with amine-containing polymers and/or proteins, especially for forming antibody conjugates with chelators, drugs, enzymes, detectable labels and the like. U.S. Pat. Nos. 5,141,648 and 5,563,250 disclose cleavable conjugates containing a labile bond that is cleavable under a variety of mild conditions. This linker is particularly useful in that the agent of interest may be bonded directly to the linker, with cleavage resulting in release of the active agent. Particular uses include adding a free amino or free sulfhydryl group to a protein, such as an antibody, or a drug.

U.S. Pat. No. 5,856,456 provides peptide linkers for use in connecting polypeptide constituents to make fusion proteins, e.g., single chain antibodies. The linker is up to about 50 amino acids in length, contains at least one occurrence of a charged amino acid (preferably arginine or lysine) followed by a proline, and is characterized by greater stability and reduced aggregation. U.S. Pat. No. 5,880,270 discloses aminooxy-containing linkers useful in a variety of immunodiagnostic and separative techniques.

### E. Multispecific Antibodies

In certain embodiments, antibodies of the present disclosure are bispecific or multispecific. Bispecific antibodies are antibodies that have binding specificities for at least two different epitopes. Exemplary bispecific antibodies may bind to two different epitopes of a single antigen. Other such antibodies may combine a first antigen binding site with a binding site for a second antigen. Alternatively, an anti-pathogen arm may be combined with an arm that binds to a triggering molecule on a leukocyte, such as a T-cell receptor molecule (e.g., CD3), or Fc receptors for IgG (FcγR), such as FcγRI (CD64), FcγRII (CD32) and Fc gamma RIII (CD16), so as to focus and localize cellular defense mechanisms to the infected cell. Bispecific antibodies may also be used to localize cytotoxic agents to infected cells. These antibodies possess a pathogen-binding arm and an arm that binds the cytotoxic agent (e.g., saporin, anti-interferon-α, vinca alkaloid, ricin A chain, methotrexate or radioactive isotope hapten). Bispecific antibodies can be prepared as full-length antibodies or antibody fragments (e.g., F(ab′)₂ bispecific antibodies). WO 96/16673 describes a bispecific anti-ErbB2/anti-Fc gamma RIII antibody and U.S. Pat. No. 5,837,234 discloses a bispecific anti-ErbB2/anti-Fc gamma RI antibody. A bispecific anti-ErbB2/Fc alpha antibody is shown in WO98/02463. U.S. Pat. No. 5,821,337 teaches a bispecific anti-ErbB2/anti-CD3 antibody.

Methods for making bispecific antibodies are known in the art. Traditional production of full-length bispecific antibodies is based on the co-expression of two immunoglobulin heavy chain-light chain pairs, where the two chains have different specificities (Millstein et al., Nature, 305:537-539 (1983)). Because of the random assortment of immunoglobulin heavy and light chains, these hybridomas (quadromas) produce a potential mixture of ten different antibody molecules, of which only one has the correct bispecific structure. Purification of the correct molecule, which is usually done by affinity chromatography steps, is rather cumbersome, and the product yields are low. Similar procedures are disclosed in WO 93/08829, and in Traunecker et al., EMBO J., 10:3655-3659 (1991).

According to a different approach, antibody variable regions with the desired binding specificities (antibody-antigen combining sites) are fused to immunoglobulin constant domain sequences. Preferably, the fusion is with an Ig heavy chain constant domain, comprising at least part of the hinge, C_{H2}, and C_{H3} regions. It is preferred to have the first heavy-chain constant region (C_{H1}) containing the site necessary for light chain bonding, present in at least one of the fusions. DNAs encoding the immunoglobulin heavy chain fusions and, if desired, the immunoglobulin light chain, are inserted into separate expression vectors, and are co-transfected into a suitable host cell. This provides for greater flexibility in adjusting the mutual proportions of the three polypeptide fragments in embodiments when unequal ratios of the three polypeptide chains used in the construction provide the optimum yield of the desired bispecific antibody. It is, however, possible to insert the coding sequences for two or all three polypeptide chains into a single expression vector when the expression of at least two polypeptide chains in equal ratios results in high yields or when the ratios have no significant effect on the yield of the desired chain combination.

In a particular embodiment of this approach, the bispecific antibodies are composed of a hybrid immunoglobulin heavy chain with a first binding specificity in one arm, and a hybrid immunoglobulin heavy chain-light chain pair (providing a second binding specificity) in the other arm. It was found that this asymmetric structure facilitates the separation of the desired bispecific compound from unwanted immunoglobulin chain combinations, as the presence of an immunoglobulin light chain in only one half of the bispecific molecule provides for a facile way of separation. This approach is disclosed in WO 94/04690. For further details of generating bispecific antibodies see, for example, Suresh et al., Methods in Enzymology, 121:210 (1986).

According to another approach described in U.S. Pat. No. 5,731,168, the interface between a pair of antibody molecules can be engineered to maximize the percentage of heterodimers that are recovered from recombinant cell culture. The preferred interface comprises at least a part of the C_{H3} domain. In this method, one or more small amino acid side chains from the interface of the first antibody molecule are replaced with larger side chains (e.g., tyrosine or tryptophan). Compensatory “cavities” of identical or similar size to the large side chain(s) are created on the interface of the second antibody molecule by replacing large amino acid side chains with smaller ones (e.g., alanine or threonine). This provides a mechanism for increasing the yield of the heterodimer over other unwanted end-products such as homodimers.

Bispecific antibodies include cross-linked or “heteroconjugate” antibodies. For example, one of the antibodies in the heteroconjugate can be coupled to avidin, the other to biotin. Such antibodies have, for example, been proposed to target immune system cells to unwanted cells (U.S. Pat. No. 4,676,980), and for treatment of HIV infection (WO 91/00360, WO 92/200373, and EP 03089). Heteroconjugate antibodies may be made using any convenient cross-linking methods. Suitable cross-linking agents are well known in the art, and are disclosed in U.S. Pat. No. 4,676,980, along with a number of cross-linking techniques.

Techniques for generating bispecific antibodies from antibody fragments have also been described in the literature. For example, bispecific antibodies can be prepared using chemical linkage. Brennan et al., Science, 229:81 (1985) describe a procedure wherein intact antibodies are proteolytically cleaved to generate F(ab′)₂ fragments. These fragments are reduced in the presence of the dithiol complexing agent, sodium arsenite, to stabilize vicinal dithiols and prevent intermolecular disulfide formation. The Fab′ fragments generated are then converted to thionitrobenzoate (TNB) derivatives. One of the Fab′-TNB derivatives is then reconverted to the Fab′-thiol by reduction with mercaptocthylamine and is mixed with an equimolar amount of the other Fab′-TNB derivative to form the bispecific antibody. The bispecific antibodies produced can be used as agents for the selective immobilization of enzymes.

Techniques exist that facilitate the direct recovery of Fab′-SH fragments from E. coli, which can be chemically coupled to form bispecific antibodies. Shalaby et al., J. Exp. Med., 175:217-225 (1992) describe the production of a humanized bispecific antibody F(ab′)₂ molecule. Each Fab′ fragment was separately secreted from E. coli and subjected to directed chemical coupling in vitro to form the bispecific antibody. The bispecific antibody thus formed was able to bind to cells overexpressing the ErbB2 receptor and normal human T cells, as well as trigger the lytic activity of human cytotoxic lymphocytes against human breast tumor targets.

Various techniques for making and isolating bispecific antibody fragments directly from recombinant cell culture have also been described (Merchant et al., Nat. Biotechnol, 16, 677-681 (1998). doi: 10.1038/nbt0798-677pmid: 9661204). For example, bispecific antibodies have been produced using leucine zippers (Kostelny et al., J. Immunol., 148 (5): 1547-1553, 1992). The leucine zipper peptides from the Fos and Jun proteins were linked to the Fab′ portions of two different antibodies by gene fusion. The antibody homodimers were reduced at the hinge region to form monomers and then re-oxidized to form the antibody heterodimers. This method can also be utilized for the production of antibody homodimers. The “diabody” technology described by Hollinger et al., Proc. Natl. Acad. Sci. USA, 90:6444-6448 (1993) has provided an alternative mechanism for making bispecific antibody fragments. The fragments comprise a V_{H} connected to a V_{L} by a linker that is too short to allow pairing between the two domains on the same chain. Accordingly, the V_{H} and V_{L} domains of one fragment are forced to pair with the complementary V_{L} and V_{H} domains of another fragment, thereby forming two antigen-binding sites. Another strategy for making bispecific antibody fragments using single-chain Fv (sFv) dimers has also been reported. See Gruber et al., J. Immunol., 152:5368 (1994).

In a particular embodiment, a bispecific or multispecific antibody may be formed as a DOCK-AND-LOCK™ (DNL™) complex (see, e.g., U.S. Pat. Nos. 7,521,056; 7,527,787; 7,534,866; 7,550,143 and 7,666,400, the Examples section of each of which is incorporated herein by reference.) Generally, the technique takes advantage of the specific and high-affinity binding interactions that occur between a dimerization and docking domain (DDD) sequence of the regulatory (R) subunits of cAMP-dependent protein kinase (PKA) and an anchor domain (AD) sequence derived from any of a variety of AKAP proteins (Baillie et al., FEBS Letters. 2005; 579: 3264; Wong and Scott, Nat. Rev. Mol. Cell Biol. 2004; 5:959). The DDD and AD peptides may be attached to any protein, peptide or other molecule. Because the DDD sequences spontaneously dimerize and bind to the AD sequence, the technique allows the formation of complexes between any selected molecules that may be attached to DDD or AD sequences.

Antibodies with more than two valencies are contemplated. For example, trispecific antibodies can be prepared (Tutt et al., J. Immunol. 147:60, 1991; Xu et al., Science, 358 (6359): 85-90, 2017). A multivalent antibody may be internalized (and/or catabolized) faster than a bivalent antibody by a cell expressing an antigen to which the antibodies bind. The antibodies of the present disclosure can be multivalent antibodies with three or more antigen binding sites (e.g., tetravalent antibodies), which can be readily produced by recombinant expression of nucleic acid encoding the polypeptide chains of the antibody. The multivalent antibody can comprise a dimerization domain and three or more antigen binding sites. The preferred dimerization domain comprises (or consists of) an Fc region or a hinge region. In this scenario, the antibody will comprise an Fc region and three or more antigen binding sites amino-terminal to the Fc region. The preferred multivalent antibody herein comprises (or consists of) three to about eight, but preferably four, antigen binding sites. The multivalent antibody comprises at least one polypeptide chain (and preferably two polypeptide chains), wherein the polypeptide chain(s) comprise two or more variable regions. For instance, the polypeptide chain(s) may comprise VD1-(X1)ₙ-VD2-(X2)ₙ-Fc, wherein VD1 is a first variable region, VD2 is a second variable region, Fc is one polypeptide chain of an Fc region, X1 and X2 represent an amino acid or polypeptide, and n is 0 or 1. For instance, the polypeptide chain(s) may comprise: VH-CH1-flexible linker-VH-CH1-Fc region chain; or VH-CH1-VH-CH1-Fc region chain. The multivalent antibody herein preferably further comprises at least two (and preferably four) light chain variable region polypeptides. The multivalent antibody herein may, for instance, comprise from about two to about eight light chain variable region polypeptides. The light chain variable region polypeptides contemplated here comprise a light chain variable region and, optionally, further comprise a C_{L} domain.

Charge modifications are particularly useful in the context of a multispecific antibody, where amino acid substitutions in Fab molecules result in reducing the mispairing of light chains with non-matching heavy chains (Bence-Jones-type side products), which can occur in the production of Fab-based bi-/multispecific antigen binding molecules with a VH/VL exchange in one (or more, in case of molecules comprising more than two antigen-binding Fab molecules) of their binding arms (see also PCT publication no. WO 2015/150447, particularly the examples therein, incorporated herein by reference in its entirety).

Accordingly, in particular embodiments, an antibody comprised in the therapeutic agent comprises:
(a) a first Fab molecule which specifically binds to a first antigen
(b) a second Fab molecule which specifically binds to a second antigen, and wherein the variable domains V_{L} and V_{H} of the Fab light chain and the Fab heavy chain are replaced by each other,
wherein the first antigen is an activating T cell antigen and the second antigen is a target cell antigen, or the first antigen is a target cell antigen and the second antigen is an activating T cell antigen; and wherein
i) in the constant domain C_{L} of the first Fab molecule under a) the amino acid at position 124 is substituted by a positively charged amino acid (numbering according to Kabat), and wherein in the constant domain CH1 of the first Fab molecule under a) the amino acid at position 147 or the amino acid at position 213 is substituted by a negatively charged amino acid (numbering according to Kabat EU index); or
ii) in the constant domain C_{L} of the second Fab molecule under b) the amino acid at position 124 is substituted by a positively charged amino acid (numbering according to Kabat), and wherein in the constant domain CH1 of the second Fab molecule under b) the amino acid at position 147 or the amino acid at position 213 is substituted by a negatively charged amino acid (numbering according to Kabat EU index).
   The antibody may not comprise both modifications mentioned under i) and ii). The constant domains CL and CH1 of the second Fab molecule are not replaced by each other (i.e., remain unexchanged).

In another embodiment of the antibody, in the constant domain CL of the first Fab molecule under a) the amino acid at position 124 is substituted independently by lysine (K), arginine (R) or histidine (H) (numbering according to Kabat) (in one preferred embodiment independently by lysine (K) or arginine (R)), and in the constant domain CH1 of the first Fab molecule under a) the amino acid at position 147 or the amino acid at position 213 is substituted independently by glutamic acid (E), or aspartic acid (D) (numbering according to Kabat EU index).

In a further embodiment, in the constant domain CL of the first Fab molecule under a) the amino acid at position 124 is substituted independently by lysine (K), arginine (R) or histidine (H) (numbering according to Kabat), and in the constant domain CH1 of the first Fab molecule under a) the amino acid at position 147 is substituted independently by glutamic acid (E), or aspartic acid (D) (numbering according to Kabat EU index).

In a particular embodiment, in the constant domain CL of the first Fab molecule under a) the amino acid at position 124 is substituted independently by lysine (K), arginine (R) or histidine (H) (numbering according to Kabat) (in one preferred embodiment independently by lysine (K) or arginine (R)) and the amino acid at position 123 is substituted independently by lysine (K), arginine (R) or histidine (H) (numbering according to Kabat) (in one preferred embodiment independently by lysine (K) or arginine (R)), and in the constant domain CH1 of the first Fab molecule under a) the amino acid at position 147 is substituted independently by glutamic acid (E), or aspartic acid (D) (numbering according to Kabat EU index) and the amino acid at position 213 is substituted independently by glutamic acid (E), or aspartic acid (D) (numbering according to Kabat EU index).

In a more particular embodiment, in the constant domain CL of the first Fab molecule under a) the amino acid at position 124 is substituted by lysine (K) (numbering according to Kabat) and the amino acid at position 123 is substituted by lysine (K) or arginine (R) (numbering according to Kabat), and in the constant domain CH1 of the first Fab molecule under a) the amino acid at position 147 is substituted by glutamic acid (E) (numbering according to Kabat EU index) and the amino acid at position 213 is substituted by glutamic acid (E) (numbering according to Kabat EU index).

In an even more particular embodiment, in the constant domain CL of the first Fab molecule under a) the amino acid at position 124 is substituted by lysine (K) (numbering according to Kabat) and the amino acid at position 123 is substituted by arginine (R) (numbering according to Kabat), and in the constant domain CH1 of the first Fab molecule under a) the amino acid at position 147 is substituted by glutamic acid (E) (numbering according to Kabat EU index) and the amino acid at position 213 is substituted by glutamic acid (E) (numbering according to Kabat EU index).

### F. Chimeric Antigen Receptors

Artificial T cell receptors (also known as chimeric T cell receptors, chimeric immunoreceptors, chimeric antigen receptors (CARs)) are engineered receptors, which graft an arbitrary specificity onto an immune effector cell. Typically, these receptors are used to graft the specificity of a monoclonal antibody onto a T cell, with transfer of their coding sequence facilitated by retroviral vectors. In this way, a large number of target-specific T cells can be generated for adoptive cell transfer. Phase I clinical studies of this approach show efficacy.

The most common form of these molecules are fusions of single-chain variable fragments (scFv) derived from monoclonal antibodies, fused to CD3-zeta transmembrane and endodomain. Such molecules result in the transmission of a zeta signal in response to recognition by the scFv of its target. An example of such a construct is 14g2a-Zeta, which is a fusion of a scFv derived from hybridoma 14g2a (which recognizes disialoganglioside GD2). When T cells express this molecule (usually achieved by oncoretroviral vector transduction), they recognize and kill target cells that express GD2 (e.g., neuroblastoma cells). To target malignant B cells, investigators have redirected the specificity of T cells using a chimeric immunoreceptor specific for the B-lineage molecule, CD19.

The variable portions of an immunoglobulin heavy and light chain are fused by a flexible linker to form a scFv. This scFv is preceded by a signal peptide to direct the nascent protein to the endoplasmic reticulum and subsequent surface expression (this is cleaved). A flexible spacer allows to the scFv to orient in different directions to enable antigen binding. The transmembrane domain is a typical hydrophobic alpha helix usually derived from the original molecule of the signaling endodomain which protrudes into the cell and transmits the desired signal.

Type I proteins are in fact two protein domains linked by a transmembrane alpha helix in between. The cell membrane lipid bilayer, through which the transmembrane domain passes, acts to isolate the inside portion (endodomain) from the external portion (ectodomain). It is not so surprising that attaching an ectodomain from one protein to an endodomain of another protein results in a molecule that combines the recognition of the former to the signal of the latter.

Ectodomain. A signal peptide directs the nascent protein into the endoplasmic reticulum. This is essential if the receptor is to be glycosylated and anchored in the cell membrane. Any eukaryotic signal peptide sequence usually works fine. Generally, the signal peptide natively attached to the amino-terminal most component is used (e.g., in a scFv with orientation light chain-linker-heavy chain, the native signal of the light-chain is used

The antigen recognition domain is usually an scFv. There are however many alternatives. An antigen recognition domain from native T-cell receptor (TCR) alpha and beta single chains have been described, as have simple ectodomains (e.g., CD4 ectodomain to recognize HIV infected cells) and more exotic recognition components such as a linked cytokine (which leads to recognition of cells bearing the cytokine receptor). In fact, almost anything that binds a given target with high affinity can be used as an antigen recognition region.

A spacer region links the antigen binding domain to the transmembrane domain. It should be flexible enough to allow the antigen binding domain to orient in different directions to facilitate antigen recognition. The simplest form is the hinge region from IgG1. Alternatives include the CH₂CH₃ region of immunoglobulin and portions of CD3. For most scFv based constructs, the IgG1 hinge suffices. However, the best spacer often has to be determined empirically.

Transmembrane domain. The transmembrane domain is a hydrophobic alpha helix that spans the membrane. Generally, the transmembrane domain from the most membrane proximal component of the endodomain is used. Interestingly, using the CD3-zeta transmembrane domain may result in incorporation of the artificial TCR into the native TCR a factor that is dependent on the presence of the native CD3-zeta transmembrane charged aspartic acid residue. Different transmembrane domains result in different receptor stability. The CD28 transmembrane domain results in a brightly expressed, stable receptor.

Endodomain. This is the “business-end” of the receptor. After antigen recognition, receptors cluster and a signal is transmitted to the cell. The most commonly used endodomain component is CD3-zeta which contains 3 ITAMs. This transmits an activation signal to the T cell after antigen is bound. CD3-zeta may not provide a fully competent activation signal and additional co-stimulatory signaling is needed.

“First generation” CARs typically had the intracellular domain from the CD3 g-chain, which is the primary transmitter of signals from endogenous TCRs. “Second generation” CARs add intracellular signaling domains from various costimulatory protein receptors (e.g., CD28, 41BB, ICOS) to the cytoplasmic tail of the CAR to provide additional signals to the T cell. Preclinical studies have indicated that the second generation of CAR designs improves the antitumor activity of T cells. More recent, “third generation” CARs combine multiple signaling domains, such as CD3z-CD28-41BB or CD3z-CD28-OX40, to further augment potency.

### G. ADCs

Antibody Drug Conjugates or ADCs are a new class of highly potent biopharmaceutical drugs designed as a targeted therapy for the treatment of people with infectious disease. ADCs are complex molecules composed of an antibody (a whole mAb or an antibody fragment such as a single-chain variable fragment, or scFv) linked, via a stable chemical linker with labile bonds, to a biological active cytotoxic/anti-viral payload or drug. Antibody Drug Conjugates are examples of bioconjugates and immunoconjugates.

By combining the unique targeting capabilities of monoclonal antibodies with the cancer-killing ability of cytotoxic drugs, antibody-drug conjugates allow sensitive discrimination between healthy and diseased tissue. This means that, in contrast to traditional systemic approaches, antibody-drug conjugates target and attack the infected cell so that healthy cells are less severely affected.

In the development ADC-based anti-tumor therapies, an anticancer drug (e.g., a cell toxin or cytotoxin) is coupled to an antibody that specifically targets a certain cell marker (e.g., a protein that, ideally, is only to be found in or on infected cells). Antibodies track these proteins down in the body and attach themselves to the surface of cancer cells. The biochemical reaction between the antibody and the target protein (antigen) triggers a signal in the tumor cell, which then absorbs or internalizes the antibody together with the cytotoxin. After the ADC is internalized, the cytotoxic drug is released and kills the cell or impairs viral replication. Due to this targeting, ideally the drug has lower side effects and gives a wider therapeutic window than other agents.

A stable link between the antibody and cytotoxic/anti-viral agent is a crucial aspect of an ADC. Linkers are based on chemical motifs including disulfides, hydrazones or peptides (cleavable), or thioethers (noncleavable) and control the distribution and delivery of the cytotoxic agent to the target cell. Cleavable and noncleavable types of linkers have been proven to be safe in preclinical and clinical trials. Brentuximab vedotin includes an enzyme-sensitive cleavable linker that delivers the potent and highly toxic antimicrotubule agent Monomethyl auristatin E or MMAE, a synthetic antineoplastic agent, to human specific CD30-positive malignant cells. Because of its high toxicity MMAE, which inhibits cell division by blocking the polymerization of tubulin, cannot be used as a single-agent chemotherapeutic drug. However, the combination of MMAE linked to an anti-CD30 monoclonal antibody (cAC10, a cell membrane protein of the tumor necrosis factor or TNF receptor) proved to be stable in extracellular fluid, cleavable by cathepsin and safe for therapy. Trastuzumab emtansine, the other approved ADC, is a combination of the microtubule-formation inhibitor mertansine (DM-1), a derivative of the Maytansine, and antibody trastuzumab (Herceptin®/Genentech/Roche) attached by a stable, non-cleavable linker. The availability of better and more stable linkers has changed the function of the chemical bond. The type of linker, cleavable or noncleavable, lends specific properties to the cytotoxic (anti-cancer) drug. For example, a non-cleavable linker keeps the drug within the cell. As a result, the entire antibody, linker and cytotoxic agent enter the targeted cancer cell where the antibody is degraded to the level of an amino acid. The resulting complex-amino acid, linker and cytotoxic agent-now becomes the active drug. In contrast, cleavable linkers are catalyzed by enzymes in the host cell where it releases the cytotoxic agent.

Another type of cleavable linker, currently in development, adds an extra molecule between the cytotoxic/anti-viral drug and the cleavage site. This linker technology allows researchers to create ADCs with more flexibility without worrying about changing cleavage kinetics. Researchers are also developing a new method of peptide cleavage based on Edman degradation, a method of sequencing amino acids in a peptide. Future direction in the development of ADCs also include the development of site-specific conjugation (TDCs) to further improve stability and therapeutic index and a emitting immunoconjugates and antibody-conjugated nanoparticles.

### H. BiTES

Bi-specific T-cell engagers (BiTEs) are a class of artificial bispecific monoclonal antibodies that are investigated for the use as anti-cancer drugs. They direct a host's immune system, more specifically the T cells' cytotoxic activity, against infected cells. BiTE is a registered trademark of Micromet AG.

BiTEs are fusion proteins consisting of two single-chain variable fragments (scFvs) of different antibodies, or amino acid sequences from four different genes, on a single peptide chain of about 55 kilodaltons. One of the scFvs binds to T cells via the CD3 receptor, and the other to an infected cell via a specific molecule.

Like other bispecific antibodies, and unlike ordinary monoclonal antibodies, BiTEs form a link between T cells and target cells. This causes T cells to exert cytotoxic/anti-viral activity on infected cells by producing proteins like perforin and granzymes, independently of the presence of MHC I or co-stimulatory molecules. These proteins enter infected cells and initiate the cell's apoptosis. This action mimics physiological processes observed during T cell attacks against infected cells.

### I. Intrabodies

In a particular embodiment, the antibody is a recombinant antibody that is suitable for action inside of a cell-such antibodies are known as “intrabodies.” These antibodies may interfere with target function by a variety of mechanism, such as by altering intracellular protein trafficking, interfering with enzymatic function, and blocking protein-protein or protein-DNA interactions. In many ways, their structures mimic or parallel those of single chain and single domain antibodies, discussed above. Indeed, single-transcript/single-chain is an important feature that permits intracellular expression in a target cell, and also makes protein transit across cell membranes more feasible. However, additional features are required.

The two major issues impacting the implementation of intrabody therapeutic are delivery, including cell/tissue targeting, and stability. With respect to delivery, a variety of approaches have been employed, such as tissue-directed delivery, use of cell-type specific promoters, viral-based delivery and use of cell-permeability/membrane translocating peptides. With respect to the stability, the approach is generally to either screen by brute force, including methods that involve phage display and may include sequence maturation or development of consensus sequences, or more directed modifications such as insertion stabilizing sequences (e.g., Fc regions, chaperone protein sequences, leucine zippers) and disulfide replacement/modification.

An additional feature that intrabodies may require is a signal for intracellular targeting. Vectors that can target intrabodies (or other proteins) to subcellular regions such as the cytoplasm, nucleus, mitochondria and ER have been designed and are commercially available (Invitrogen Corp.; Persic et al., 1997).

By virtue of their ability to enter cells, intrabodies have additional uses that other types of antibodies may not achieve. In the case of the present antibodies, the ability to interact with the MUC1 cytoplasmic domain in a living cell may interfere with functions associated with the MUC1 CD, such as signaling functions (binding to other molecules) or oligomer formation. In particular, it is contemplated that such antibodies can be used to inhibit MUC1 dimer formation.

### J. Purification

In certain embodiments, the antibodies of the present disclosure may be purified. The term “purified,” as used herein, is intended to refer to a composition, isolatable from other components, wherein the protein is purified to any degree relative to its naturally obtainable state. A purified protein therefore also refers to a protein, free from the environment in which it may naturally occur. Where the term “substantially purified” is used, this designation will refer to a composition in which the protein or peptide forms the major component of the composition, such as constituting about 50%, about 60%, about 70%, about 80%, about 90%, about 95% or more of the proteins in the composition.

Protein purification techniques are well known to those of skill in the art. These techniques involve, at one level, the crude fractionation of the cellular milieu to polypeptide and non-polypeptide fractions. Having separated the polypeptide from other proteins, the polypeptide of interest may be further purified using chromatographic and electrophoretic techniques to achieve partial or complete purification (or purification to homogeneity). Analytical methods particularly suited to the preparation of a pure peptide are ion-exchange chromatography, exclusion chromatography; polyacrylamide gel electrophoresis; isoelectric focusing. Other methods for protein purification include precipitation with ammonium sulfate, PEG, antibodies and the like or by heat denaturation, followed by centrifugation; gel filtration, reverse phase, hydroxylapatite and affinity chromatography; and combinations of such and other techniques.

In purifying an antibody of the present disclosure, it may be desirable to express the polypeptide in a prokaryotic or eukaryotic expression system and extract the protein using denaturing conditions. The polypeptide may be purified from other cellular components using an affinity column, which binds to a tagged portion of the polypeptide. As is generally known in the art, it is believed that the order of conducting the various purification steps may be changed, or that certain steps may be omitted, and still result in a suitable method for the preparation of a substantially purified protein or peptide.

Commonly, complete antibodies are fractionated utilizing agents (i.e., protein A) that bind the Fc portion of the antibody. Alternatively, antigens may be used to simultaneously purify and select appropriate antibodies. Such methods often utilize the selection agent bound to a support, such as a column, filter or bead. The antibodies are bound to a support, contaminants removed (e.g., washed away), and the antibodies released by applying conditions (salt, heat, etc.).

Various methods for quantifying the degree of purification of the protein or peptide will be known to those of skill in the art in light of the present disclosure. These include, for example, determining the specific activity of an active fraction, or assessing the amount of polypeptides within a fraction by SDS/PAGE analysis. Another method for assessing the purity of a fraction is to calculate the specific activity of the fraction, to compare it to the specific activity of the initial extract, and to thus calculate the degree of purity. The actual units used to represent the amount of activity will, of course, be dependent upon the particular assay technique chosen to follow the purification and whether or not the expressed protein or peptide exhibits a detectable activity.

It is known that the migration of a polypeptide can vary, sometimes significantly, with different conditions of SDS/PAGE (Capaldi et al., 1977). It will therefore be appreciated that under differing electrophoresis conditions, the apparent molecular weights of purified or partially purified expression products may vary.

### III. Active/Passive Immunization and Treatment/Prevention of EEEV Infection

### A. Formulation and Administration

The present disclosure provides pharmaceutical compositions comprising anti-EEEV antibodies and antigens for generating the same. Such compositions comprise a prophylactically or therapeutically effective amount of an antibody or a fragment thereof, or a peptide immunogen, and a pharmaceutically acceptable carrier. In a specific embodiment, the term “pharmaceutically acceptable” means approved by a regulatory agency of the Federal or a state government or listed in the U.S. Pharmacopeia or other generally recognized pharmacopeia for use in animals, and more particularly in humans. The term “carrier” refers to a diluent, excipient, or vehicle with which the therapeutic is administered. Such pharmaceutical carriers can be sterile liquids, such as water and oils, including those of petroleum, animal, vegetable or synthetic origin, such as peanut oil, soybean oil, mineral oil, sesame oil and the like. Water is a particular carrier when the pharmaceutical composition is administered intravenously. Saline solutions and aqueous dextrose and glycerol solutions can also be employed as liquid carriers, particularly for injectable solutions. Other suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like.

The composition, if desired, can also contain minor amounts of wetting or emulsifying agents, or pH buffering agents. These compositions can take the form of solutions, suspensions, emulsion, tablets, pills, capsules, powders, sustained-release formulations and the like. Oral formulations can include standard carriers such as pharmaceutical grades of mannitol, lactose, starch, magnesium stearate, sodium saccharine, cellulose, magnesium carbonate, etc. Examples of suitable pharmaceutical agents are described in “Remington's Pharmaceutical Sciences.” Such compositions will contain a prophylactically or therapeutically effective amount of the antibody or fragment thereof, preferably in purified form, together with a suitable amount of carrier so as to provide the form for proper administration to the patient. The formulation should suit the mode of administration, which can be oral, intravenous, intraarterial, intrabuccal, intranasal, nebulized, bronchial inhalation, intra-rectal, vaginal, topical or delivered by mechanical ventilation.

Active vaccines are also envisioned where antibodies like those disclosed are produced in vivo in a subject at risk of EEEV infection. Such vaccines can be formulated for parenteral administration, e.g., formulated for injection via the intradermal, intravenous, intramuscular, subcutaneous, or even intraperitoneal routes. Administration by intradermal and intramuscular routes are contemplated. The vaccine could alternatively be administered by a topical route directly to the mucosa, for example, by nasal drops, inhalation, by nebulizer, or via intrarectal or vaginal delivery. Pharmaceutically acceptable salts include the acid salts and those which are formed with inorganic acids such as, for example, hydrochloric or phosphoric acids, or such organic acids as acetic, oxalic, tartaric, mandelic, and the like. Salts formed with the free carboxyl groups may also be derived from inorganic bases such as, for example, sodium, potassium, ammonium, calcium, or ferric hydroxides, and such organic bases as isopropylamine, trimethylamine, 2-ethylamino ethanol, histidine, procaine, and the like.

Passive transfer of antibodies, known as artificially acquired passive immunity, generally will involve the use of intravenous or intramuscular injections. The forms of antibody can be human or animal blood plasma or serum, as pooled human immunoglobulin for intravenous (IVIG) or intramuscular (IG) use, as high-titer human IVIG or IG from immunized or from donors recovering from disease, and as monoclonal antibodies (MAb). Such immunity generally lasts for only a short period of time, and there is also a potential risk for hypersensitivity reactions, and serum sickness, especially from gamma globulin of non-human origin. However, passive immunity provides immediate protection. The antibodies will be formulated in a carrier suitable for injection, i.e., sterile and syringeable.

Generally, the ingredients of compositions of the disclosure are supplied either separately or mixed together in unit dosage form, for example, as a dry lyophilized powder or water-free concentrate in a hermetically sealed container such as an ampoule or sachette indicating the quantity of active agent. Where the composition is to be administered by infusion, it can be dispensed with an infusion bottle containing sterile pharmaceutical grade water or saline. Where the composition is administered by injection, an ampoule of sterile water for injection or saline can be provided so that the ingredients may be mixed prior to administration.

The compositions of the disclosure can be formulated as neutral or salt forms. Pharmaceutically acceptable salts include those formed with anions such as those derived from hydrochloric, phosphoric, acetic, oxalic, tartaric acids, etc., and those formed with cations such as those derived from sodium, potassium, ammonium, calcium, ferric hydroxides, isopropylamine, triethylamine, 2-ethylamino ethanol, histidine, procaine, etc.

### B. ADCC

Antibody-dependent cell-mediated cytotoxicity (ADCC) is an immune mechanism leading to the lysis of antibody-coated target cells by immune effector cells. The target cells are cells to which antibodies or fragments thereof comprising an Fc region specifically bind, generally via the protein part that is N-terminal to the Fc region. By “antibody having increased/reduced antibody dependent cell-mediated cytotoxicity (ADCC)” is meant an antibody having increased/reduced ADCC as determined by any suitable method known to those of ordinary skill in the art.

As used herein, the term “increased/reduced ADCC” is defined as either an increase/reduction in the number of target cells that are lysed in a given time, at a given concentration of antibody in the medium surrounding the target cells, by the mechanism of ADCC defined above, and/or a reduction/increase in the concentration of antibody, in the medium surrounding the target cells, required to achieve the lysis of a given number of target cells in a given time, by the mechanism of ADCC. The increase/reduction in ADCC is relative to the ADCC mediated by the same antibody produced by the same type of host cells, using the same standard production, purification, formulation and storage methods (which are known to those skilled in the art), but that has not been engineered. For example, the increase in ADCC mediated by an antibody produced by host cells engineered to have an altered pattern of glycosylation (e.g., to express the glycosyltransferase, GnTIII, or other glycosyltransferases) by the methods described herein, is relative to the ADCC mediated by the same antibody produced by the same type of non-engineered host cells.

### C. CDC

Complement-dependent cytotoxicity (CDC) is a function of the complement system. It is the processes in the immune system that kill pathogens by damaging their membranes without the involvement of antibodies or cells of the immune system. There are three main processes. All three insert one or more membrane attack complexes (MAC) into the pathogen that cause lethal colloid-osmotic swelling, i.e., CDC. It is one of the mechanisms by which antibodies or antibody fragments have an anti-viral effect.

### IV. Antibody Conjugates

Antibodies of the present disclosure may be linked to at least one agent to form an antibody conjugate. To increase the efficacy of antibody molecules as diagnostic or therapeutic agents, it is conventional to link or covalently bind or complex at least one desired molecule or moiety. Such a molecule or moiety may be, but is not limited to, at least one effector or reporter molecule. Effector molecules comprise molecules having a desired activity, e.g., cytotoxic activity. Non-limiting examples of effector molecules which have been attached to antibodies include toxins, anti-tumor agents, therapeutic enzymes, radionuclides, antiviral agents, chelating agents, cytokines, growth factors, and oligo- or polynucleotides. By contrast, a reporter molecule is defined as any moiety which may be detected using an assay. Non-limiting examples of reporter molecules which have been conjugated to antibodies include enzymes, radiolabels, haptens, fluorescent labels, phosphorescent molecules, chemiluminescent molecules, chromophores, photoaffinity molecules, colored particles or ligands, such as biotin.

Antibody conjugates are generally preferred for use as diagnostic agents. Antibody diagnostics generally fall within two classes, those for use in in vitro diagnostics, such as in a variety of immunoassays, and those for use in vivo diagnostic protocols, generally known as “antibody-directed imaging.” Many appropriate imaging agents are known in the art, as are methods for their attachment to antibodies (see, for e.g., U.S. Pat. Nos. 5,021,236, 4,938,948, and 4,472,509). The imaging moieties used can be paramagnetic ions, radioactive isotopes, fluorochromes, NMR-detectable substances, and X-ray imaging agents.

In the case of paramagnetic ions, one might mention by way of example ions such as chromium (III), manganese (II), iron (III), iron (II), cobalt (II), nickel (II), copper (II), neodymium (III), samarium (III), ytterbium (III), gadolinium (III), vanadium (II), terbium (III), dysprosium (III), holmium (III) and/or erbium (III), with gadolinium being particularly preferred. Ions useful in other contexts, such as X-ray imaging, include but are not limited to lanthanum (III), gold (III), lead (II), and especially bismuth (III).

In the case of radioactive isotopes for therapeutic and/or diagnostic application, one might mention astatine²¹¹, ¹⁴carbon, ⁵¹chromium, ³⁶chlorine, ⁵⁷cobalt, ⁵⁸ cobalt, copper⁶⁷, ¹⁵²Eu, gallium⁶⁷, ³hydrogen, iodine¹²³, iodine¹²⁵, iodine¹³¹, indium ¹¹¹, ⁵⁹iron, ³²phosphorus, rhenium¹⁸⁶, rhenium¹⁸⁸, ⁷⁵selenium, ³⁵sulphur, technicium^{99m} and/or yttrium ⁹⁰. ¹²⁵I is often being preferred for use in certain embodiments, and technicium^{99m} and/or indium¹¹¹ are also often preferred due to their low energy and suitability for long range detection. Radioactively labeled monoclonal antibodies of the present disclosure may be produced according to well-known methods in the art. For instance, monoclonal antibodies can be iodinated by contact with sodium and/or potassium iodide and a chemical oxidizing agent such as sodium hypochlorite, or an enzymatic oxidizing agent, such as lactoperoxidase. Monoclonal antibodies according to the disclosure may be labeled with technetium^{99m} by ligand exchange process, for example, by reducing pertechnate with stannous solution, chelating the reduced technetium onto a Sephadex column and applying the antibody to this column. Alternatively, direct labeling techniques may be used, e.g., by incubating pertechnate, a reducing agent such as SNCl₂, a buffer solution such as sodium-potassium phthalate solution, and the antibody. Intermediary functional groups which are often used to bind radioisotopes which exist as metallic ions to antibody are diethylenetriaminepentaacetic acid (DTPA) or ethylene diaminetetracetic acid (EDTA).

Among the fluorescent labels contemplated for use as conjugates include Alexa 350, Alexa 430, AMCA, BODIPY 630/650, BODIPY 650/665, BODIPY-FL, BODIPY-R6G, BODIPY-TMR, BODIPY-TRX, Cascade Blue, Cy3, Cy5,6-FAM, Fluorescein Isothiocyanate, HEX, 6-JOE, Oregon Green 488, Oregon Green 500, Oregon Green 514, Pacific Blue, REG, Rhodamine Green, Rhodamine Red, Renographin, ROX, TAMRA, TET, Tetramethylrhodamine, and/or Texas Red.

Additional types of antibodies contemplated in the present disclosure are those intended primarily for use in vitro, where the antibody is linked to a secondary binding ligand and/or to an enzyme (an enzyme tag) that will generate a colored product upon contact with a chromogenic substrate. Examples of suitable enzymes include urease, alkaline phosphatase, (horseradish) hydrogen peroxidase or glucose oxidase. Preferred secondary binding ligands are biotin and avidin and streptavidin compounds. The use of such labels is well known to those of skill in the art and are described, for example, in U.S. Pat. Nos. 3,817,837, 3,850,752, 3,939,350, 3,996,345, 4,277,437, 4,275,149 and 4,366,241.

Yet another known method of site-specific attachment of molecules to antibodies comprises the reaction of antibodies with hapten-based affinity labels. Essentially, hapten-based affinity labels react with amino acids in the antigen binding site, thereby destroying this site and blocking specific antigen reaction. However, this may not be advantageous since it results in loss of antigen binding by the antibody conjugate.

Molecules containing azido groups may also be used to form covalent bonds to proteins through reactive nitrene intermediates that are generated by low intensity ultraviolet light (Potter and Haley, 1983). In particular, 2- and 8-azido analogues of purine nucleotides have been used as site-directed photoprobes to identify nucleotide binding proteins in crude cell extracts (Owens & Haley, 1987; Atherton et al., 1985). The 2- and 8-azido nucleotides have also been used to map nucleotide binding domains of purified proteins (Khatoon et al., 1989; King et al., 1989; Dholakia et al., 1989) and may be used as antibody binding agents.

Several methods are known in the art for the attachment or conjugation of an antibody to its conjugate moiety. Some attachment methods involve the use of a metal chelate complex employing, for example, an organic chelating agent such a diethylenetriaminepentaacetic acid anhydride (DTPA); ethylenetriaminetetraacetic acid; N-chloro-p-toluenesulfonamide; and/or tetrachloro-3α-6α-diphenylglycouril-3 attached to the antibody (U.S. Pat. Nos. 4,472,509 and 4,938,948). Monoclonal antibodies may also be reacted with an enzyme in the presence of a coupling agent such as glutaraldehyde or periodate. Conjugates with fluorescein markers are prepared in the presence of these coupling agents or by reaction with an isothiocyanate. In U.S. Pat. No. 4,938,948, imaging of breast tumors is achieved using monoclonal antibodies and the detectable imaging moieties are bound to the antibody using linkers such as methyl-p-hydroxybenzimidate or N-succinimidyl-3-(4-hydroxyphenyl) propionate.

In other embodiments, derivatization of immunoglobulins by selectively introducing sulfhydryl groups in the Fc region of an immunoglobulin, using reaction conditions that do not alter the antibody combining site are contemplated. Antibody conjugates produced according to this methodology are disclosed to exhibit improved longevity, specificity and sensitivity (U.S. Pat. No. 5,196,066, incorporated herein by reference). Site-specific attachment of effector or reporter molecules, wherein the reporter or effector molecule is conjugated to a carbohydrate residue in the Fc region have also been disclosed in the literature (O'Shannessy et al., 1987). This approach has been reported to produce diagnostically and therapeutically promising antibodies which are currently in clinical evaluation.

### V. Immunodetection Methods

In still further embodiments, the present disclosure concerns immunodetection methods for binding, purifying, removing, quantifying and otherwise generally detecting EEEV and its associated antigens. While such methods can be applied in a traditional sense, another use will be in quality control and monitoring of vaccine and other virus stocks, where antibodies according to the present disclosure can be used to assess the amount or integrity (i.e., long term stability) of antigens in viruses. Alternatively, the methods may be used to screen various antibodies for appropriate/desired reactivity profiles.

Other immunodetection methods include specific assays for determining the presence of EEEV in a subject. A wide variety of assay formats are contemplated, but specifically those that would be used to detect EEEV in a fluid obtained from a subject, such as saliva, blood, plasma, sputum, semen or urine. In particular, semen has been demonstrated as a viable sample for detecting viruses in general (Purpura et al., 2016; Mansuy et al., 2016; Barzon et al., 2016; Gornet et al., 2016; Duffy et al., 2009; CDC, 2016; Halfon et al., 2010; Elder et al. 2005). The assays may be advantageously formatted for non-healthcare (home) use, including lateral flow assays (see below) analogous to home pregnancy tests. These assays may be packaged in the form of a kit with appropriate reagents and instructions to permit use by the subject of a family member.

Some immunodetection methods include enzyme linked immunosorbent assay (ELISA), radioimmunoassay (RIA), immunoradiometric assay, fluoroimmunoassay, chemiluminescent assay, bioluminescent assay, and Western blot to mention a few. In particular, a competitive assay for the detection and quantitation of EEEV antibodies directed to specific parasite epitopes in samples also is provided. The steps of various useful immunodetection methods have been described in the scientific literature, such as, e.g., Doolittle and Ben-Zeev (1999), Gulbis and Galand (1993), De Jager et al. (1993), and Nakamura et al. (1987). In general, the immunobinding methods include obtaining a sample suspected of containing EEEV and contacting the sample with a first antibody in accordance with the present disclosure, as the case may be, under conditions effective to allow the formation of immunocomplexes.

These methods include methods for purifying EEEV or related antigens from a sample. The antibody will preferably be linked to a solid support, such as in the form of a column matrix, and the sample suspected of containing the EEEV or antigenic component will be applied to the immobilized antibody. The unwanted components will be washed from the column, leaving the EEEV antigen immunocomplexed to the immobilized antibody, which is then collected by removing the organism or antigen from the column.

The immunobinding methods also include methods for detecting and quantifying the amount of EEEV or related components in a sample and the detection and quantification of any immune complexes formed during the binding process. Here, one would obtain a sample suspected of containing EEEV or its antigens and contact the sample with an antibody that binds EEEV or components thereof, followed by detecting and quantifying the amount of immune complexes formed under the specific conditions. In terms of antigen detection, the biological sample analyzed may be any sample that is suspected of containing EEEV or EEEV antigen, such as a tissue section or specimen, a homogenized tissue extract, a biological fluid, including blood and serum, or a secretion, such as feces or urine.

Contacting the chosen biological sample with the antibody under effective conditions and for a period of time sufficient to allow the formation of immune complexes (primary immune complexes) is generally a matter of simply adding the antibody composition to the sample and incubating the mixture for a period of time long enough for the antibodies to form immune complexes with, i.e., to bind to EEEV or antigens present. After this time, the sample-antibody composition, such as a tissue section, ELISA plate, dot blot or Western blot, will generally be washed to remove any non-specifically bound antibody species, allowing only those antibodies specifically bound within the primary immune complexes to be detected.

In general, the detection of immunocomplex formation is well known in the art and may be achieved through the application of numerous approaches. These methods are generally based upon the detection of a label or marker, such as any of those radioactive, fluorescent, biological and enzymatic tags. Patents concerning the use of such labels include U.S. Pat. Nos. 3,817,837, 3,850,752, 3,939,350, 3,996,345, 4,277,437, 4,275,149 and 4,366,241. Of course, one may find additional advantages using a secondary binding ligand such as a second antibody and/or a biotin/avidin ligand binding arrangement, as is known in the art.

The antibody employed in the detection may itself be linked to a detectable label, wherein one would then simply detect this label, thereby allowing the amount of the primary immune complexes in the composition to be determined. Alternatively, the first antibody that becomes bound within the primary immune complexes may be detected by means of a second binding ligand that has binding affinity for the antibody. In these cases, the second binding ligand may be linked to a detectable label. The second binding ligand is itself often an antibody, which may thus be termed a “secondary” antibody. The primary immune complexes are contacted with the labeled, secondary binding ligand, or antibody, under effective conditions and for a period sufficient to allow the formation of secondary immune complexes. The secondary immune complexes are then generally washed to remove any non-specifically bound labeled secondary antibodies or ligands, and the remaining label in the secondary immune complexes is then detected.

Further methods include the detection of primary immune complexes by a two-step approach. A second binding ligand, such as an antibody that has binding affinity for the antibody, is used to form secondary immune complexes, as described above. After washing, the secondary immune complexes are contacted with a third binding ligand or antibody that has binding affinity for the second antibody, again under effective conditions and for a period of time sufficient to allow the formation of immune complexes (tertiary immune complexes). The third ligand or antibody is linked to a detectable label, allowing detection of the tertiary immune complexes thus formed. This system may provide for signal amplification if this is desired.

One method of immunodetection uses two different antibodies. A first biotinylated antibody is used to detect the target antigen, and a second antibody is then used to detect the biotin attached to the complexed biotin. In that method, the sample to be tested is first incubated in a solution containing the first step antibody. If the target antigen is present, some of the antibody binds to the antigen to form a biotinylated antibody/antigen complex. The antibody/antigen complex is then amplified by incubation in successive solutions of streptavidin (or avidin), biotinylated DNA, and/or complementary biotinylated DNA, with each step adding additional biotin sites to the antibody/antigen complex. The amplification steps are repeated until a suitable level of amplification is achieved, at which point the sample is incubated in a solution containing the second step antibody against biotin. This second step antibody is labeled, for example, with an enzyme that can be used to detect the presence of the antibody/antigen complex by histoenzymology using a chromogen substrate. With suitable amplification, a conjugate can be produced which is macroscopically visible.

Another known method of immunodetection takes advantage of the immuno-PCR (Polymerase Chain Reaction) methodology. The PCR method is similar to the Cantor method up to the incubation with biotinylated DNA, however, instead of using multiple rounds of streptavidin and biotinylated DNA incubation, the DNA/biotin/streptavidin/antibody complex is washed out with a low pH or high salt buffer that releases the antibody. The resulting wash solution is then used to carry out a PCR reaction with suitable primers with appropriate controls. At least in theory, the enormous amplification capability and specificity of PCR can be utilized to detect a single antigen molecule.

### A. ELISAs

Immunoassays, in their most simple and direct sense, are binding assays. Certain preferred immunoassays are the various types of enzyme-linked immunosorbent assays (ELISAs) and radioimmunoassays (RIA) known in the art. Immunohistochemical detection using tissue sections is also particularly useful. However, it will be readily appreciated that detection is not limited to such techniques, and western blotting, dot blotting, FACS analyses, and the like may also be used.

In one exemplary ELISA, the antibodies of the disclosure are immobilized onto a selected surface exhibiting protein affinity, such as a well in a polystyrene microtiter plate. Then, a test composition suspected of containing the EEEV or EEEV antigen is added to the wells. After binding and washing to remove non-specifically bound immune complexes, the bound antigen may be detected. Detection may be achieved by the addition of another anti-EEEV antibody that is linked to a detectable label. This type of ELISA is a simple “sandwich ELISA.” Detection may also be achieved by the addition of a second anti-EEEV antibody, followed by the addition of a third antibody that has binding affinity for the second antibody, with the third antibody being linked to a detectable label.

In another exemplary ELISA, the samples suspected of containing the EEEV or EEEV antigen are immobilized onto the well surface and then contacted with the anti-EEEV antibodies of the disclosure. After binding and washing to remove non-specifically bound immune complexes, the bound anti-EEEV antibodies are detected. Where the initial anti-EEEV antibodies are linked to a detectable label, the immune complexes may be detected directly. Again, the immune complexes may be detected using a second antibody that has binding affinity for the first anti-EEEV antibody, with the second antibody being linked to a detectable label.

Irrespective of the format employed, ELISAs have certain features in common, such as coating, incubating and binding, washing to remove non-specifically bound species, and detecting the bound immune complexes. These are described below.

In coating a plate with either antigen or antibody, one will generally incubate the wells of the plate with a solution of the antigen or antibody, either overnight or for a specified period of hours. The wells of the plate will then be washed to remove incompletely adsorbed material. Any remaining available surfaces of the wells are then “coated” with a nonspecific protein that is antigenically neutral with regard to the test antisera. These include bovine serum albumin (BSA), casein or solutions of milk powder. The coating allows for blocking of nonspecific adsorption sites on the immobilizing surface and thus reduces the background caused by nonspecific binding of antisera onto the surface.

In ELISAs, it is probably more customary to use a secondary or tertiary detection means rather than a direct procedure. Thus, after binding of a protein or antibody to the well, coating with a non-reactive material to reduce background, and washing to remove unbound material, the immobilizing surface is contacted with the biological sample to be tested under conditions effective to allow immune complex (antigen/antibody) formation. Detection of the immune complex then requires a labeled secondary binding ligand or antibody, and a secondary binding ligand or antibody in conjunction with a labeled tertiary antibody or a third binding ligand.

“Under conditions effective to allow immune complex (antigen/antibody) formation” means that the conditions preferably include diluting the antigens and/or antibodies with solutions such as BSA, bovine gamma globulin (BGG) or phosphate buffered saline (PBS)/Tween. These added agents also tend to assist in the reduction of nonspecific background.

The “suitable” conditions also mean that the incubation is at a temperature or for a period of time sufficient to allow effective binding. Incubation steps are typically from about 1 to 2 to 4 hours or so, at temperatures preferably on the order of 25° C. to 27° C. or may be overnight at about 4° C. or so.

Following all incubation steps in an ELISA, the contacted surface is washed so as to remove non-complexed material. A preferred washing procedure includes washing with a solution such as PBS/Tween, or borate buffer. Following the formation of specific immune complexes between the test sample and the originally bound material, and subsequent washing, the occurrence of even minute amounts of immune complexes may be determined.

To provide a detecting means, the second or third antibody will have an associated label to allow detection. Preferably, this will be an enzyme that will generate color development upon incubating with an appropriate chromogenic substrate. Thus, for example, one will desire to contact or incubate the first and second immune complex with a urease, glucose oxidase, alkaline phosphatase or hydrogen peroxidase-conjugated antibody for a period of time and under conditions that favor the development of further immune complex formation (e.g., incubation for 2 hours at room temperature in a PBS-containing solution such as PBS-Tween).

After incubation with the labeled antibody, and subsequent to washing to remove unbound material, the amount of label is quantified, e.g., by incubation with a chromogenic substrate such as urea, or bromocresol purple, or 2,2′-azino-di-(3-ethyl-benzthiazoline-6-sulfonic acid (ABTS), or H₂O₂, in the case of peroxidase as the enzyme label. Quantification is then achieved by measuring the degree of color generated, e.g., using a visible spectra spectrophotometer.

In another embodiment, the present disclosure contemplates the use of competitive formats. This is particularly useful in the detection of EEEV antibodies in sample. In competition-based assays, an unknown amount of analyte or antibody is determined by its ability to displace a known amount of labeled antibody or analyte. Thus, the quantifiable loss of a signal is an indication of the amount of unknown antibody or analyte in a sample.

Here, the inventor proposes the use of labeled EEEV monoclonal antibodies to determine the amount of EEEV antibodies in a sample. The basic format would include contacting a known amount of EEEV monoclonal antibody (linked to a detectable label) with EEEV antigen or particle. The EEEV antigen or organism is preferably attached to a support. After binding of the labeled monoclonal antibody to the support, the sample is added and incubated under conditions permitting any unlabeled antibody in the sample to compete with, and hence displace, the labeled monoclonal antibody. By measuring either the lost label or the label remaining (and subtracting that from the original amount of bound label), one can determine how much non-labeled antibody is bound to the support, and thus how much antibody was present in the sample.

### B. Western Blot

The Western blot (alternatively, protein immunoblot) is an analytical technique used to detect specific proteins in a given sample of tissue homogenate or extract. It uses gel electrophoresis to separate native or denatured proteins by the length of the polypeptide (denaturing conditions) or by the 3-D structure of the protein (native/non-denaturing conditions). The proteins are then transferred to a membrane (typically nitrocellulose or PVDF), where they are probed (detected) using antibodies specific to the target protein.

Samples may be taken from whole tissue or from cell culture. In most cases, solid tissues are first broken down mechanically using a blender (for larger sample volumes), using a homogenizer (smaller volumes), or by sonication. Cells may also be broken open by one of the above mechanical methods. However, it should be noted that bacteria, virus or environmental samples can be the source of protein and thus Western blotting is not restricted to cellular studies only. Assorted detergents, salts, and buffers may be employed to encourage lysis of cells and to solubilize proteins. Protease and phosphatase inhibitors are often added to prevent the digestion of the sample by its own enzymes. Tissue preparation is often done at cold temperatures to avoid protein denaturing.

The proteins of the sample are separated using gel electrophoresis. Separation of proteins may be by isoelectric point (pI), molecular weight, electric charge, or a combination of these factors. The nature of the separation depends on the treatment of the sample and the nature of the gel. This is a very useful way to determine a protein. It is also possible to use a two-dimensional (2-D) gel which spreads the proteins from a single sample out in two dimensions. Proteins are separated according to isoelectric point (pH at which they have neutral net charge) in the first dimension, and according to their molecular weight in the second dimension.

In order to make the proteins accessible to antibody detection, they are moved from within the gel onto a membrane made of nitrocellulose or polyvinylidene difluoride (PVDF). The membrane is placed on top of the gel, and a stack of filter papers placed on top of that. The entire stack is placed in a buffer solution which moves up the paper by capillary action, bringing the proteins with it. Another method for transferring the proteins is called electroblotting and uses an electric current to pull proteins from the gel into the PVDF or nitrocellulose membrane. The proteins move from within the gel onto the membrane while maintaining the organization they had within the gel. As a result of this blotting process, the proteins are exposed on a thin surface layer for detection (see below). Both varieties of membrane are chosen for their non-specific protein binding properties (i.e., binds all proteins equally well). Protein binding is based upon hydrophobic interactions, as well as charged interactions between the membrane and protein. Nitrocellulose membranes are cheaper than PVDF but are far more fragile and do not stand up well to repeated probings. The uniformity and overall effectiveness of transfer of protein from the gel to the membrane can be checked by staining the membrane with Coomassie Brilliant Blue or Ponceau S dyes. Once transferred, proteins are detected using labeled primary antibodies, or unlabeled primary antibodies followed by indirect detection using labeled protein A or secondary labeled antibodies binding to the Fc region of the primary antibodies.

### C. Lateral Flow Assays

Lateral flow assays, also known as lateral flow immunochromatographic assays, are simple devices intended to detect the presence (or absence) of a target analyte in sample (matrix) without the need for specialized and costly equipment, though many laboratory-based applications exist that are supported by reading equipment. Typically, these tests are used as low resources medical diagnostics, either for home testing, point of care testing, or laboratory use. A widely spread and well-known application is the home pregnancy test.

The technology is based on a series of capillary beds, such as pieces of porous paper or sintered polymer. Each of these elements has the capacity to transport fluid (e.g., urine) spontaneously. The first element (the sample pad) acts as a sponge and holds an excess of sample fluid. Once soaked, the fluid migrates to the second element (conjugate pad) in which the manufacturer has stored the so-called conjugate, a dried format of bio-active particles (see below) in a salt-sugar matrix that contains everything to guarantee an optimized chemical reaction between the target molecule (e.g., an antigen) and its chemical partner (e.g., antibody) that has been immobilized on the particle's surface. While the sample fluid dissolves the salt-sugar matrix, it also dissolves the particles and in one combined transport action the sample and conjugate mix while flowing through the porous structure. In this way, the analyte binds to the particles while migrating further through the third capillary bed. This material has one or more areas (often called stripes) where a third molecule has been immobilized by the manufacturer. By the time the sample-conjugate mix reaches these strips, analyte has been bound on the particle and the third ‘capture’ molecule binds the complex. After a while, when more and more fluid has passed the stripes, particles accumulate and the stripe-area changes color. Typically, there are at least two stripes: one (the control) that captures any particle and thereby shows that reaction conditions and technology worked fine, the second contains a specific capture molecule and only captures those particles onto which an analyte molecule has been immobilized. After passing these reaction zones, the fluid enters the final porous material—the wick—that simply acts as a waste container. Lateral Flow Tests can operate as either competitive or sandwich assays. Lateral flow assays are disclosed in U.S. Pat. No. 6,485,982.

### D. Immunohistochemistry

The antibodies of the present disclosure may also be used in conjunction with both fresh-frozen and/or formalin-fixed, paraffin-embedded tissue blocks prepared for study by immunohistochemistry (IHC). The method of preparing tissue blocks from these particulate specimens has been successfully used in previous IHC studies of various prognostic factors and is well known to those of skill in the art (Brown et al., 1990; Abbondanzo et al., 1990; Allred et al., 1990).

Briefly, frozen-sections may be prepared by rehydrating 50 ng of frozen “pulverized” tissue at room temperature in phosphate buffered saline (PBS) in small plastic capsules; pelleting the particles by centrifugation; resuspending them in a viscous embedding medium (OCT); inverting the capsule and/or pelleting again by centrifugation; snap-freezing in −70° C. isopentane; cutting the plastic capsule and/or removing the frozen cylinder of tissue; securing the tissue cylinder on a cryostat microtome chuck; and/or cutting 25-50 serial sections from the capsule. Alternatively, whole frozen tissue samples may be used for serial section cuttings.

Permanent-sections may be prepared by a similar method involving rehydration of the 50 mg sample in a plastic microfuge tube; pelleting; resuspending in 10% formalin for 4 hours fixation; washing/pelleting; resuspending in warm 2.5% agar; pelleting; cooling in ice water to harden the agar; removing the tissue/agar block from the tube; infiltrating and/or embedding the block in paraffin; and/or cutting up to 50 serial permanent sections. Again, whole tissue samples may be substituted.

### E. Immunodetection Kits

In still further embodiments, the present disclosure concerns immunodetection kits for use with the immunodetection methods described above. As the antibodies may be used to detect EEEV or EEEV antigens, the antibodies may be included in the kit. The immunodetection kits will thus comprise, in suitable container means, a first antibody that binds to EEEV or EEEV antigen, and optionally an immunodetection reagent.

In certain embodiments, the EEEV antibody may be pre-bound to a solid support, such as a column matrix and/or well of a microtiter plate. The immunodetection reagents of the kit may take any one of a variety of forms, including those detectable labels that are associated with or linked to the given antibody. Detectable labels that are associated with or attached to a secondary binding ligand are also contemplated. Exemplary secondary ligands are those secondary antibodies that have binding affinity for the first antibody.

Further suitable immunodetection reagents for use in the present kits include the two-component reagent that comprises a secondary antibody that has binding affinity for the first antibody, along with a third antibody that has binding affinity for the second antibody, the third antibody being linked to a detectable label. As noted above, a number of exemplary labels are known in the art and all such labels may be employed in connection with the present disclosure.

The kits may further comprise a suitably aliquoted composition of the EEEV or EEEV antigens, whether labeled or unlabeled, as may be used to prepare a standard curve for a detection assay. The kits may contain antibody-label conjugates either in fully conjugated form, in the form of intermediates, or as separate moieties to be conjugated by the user of the kit. The components of the kits may be packaged either in aqueous media or in lyophilized form.

The container means of the kits will generally include at least one vial, test tube, flask, bottle, syringe or other container means, into which the antibody may be placed, or preferably, suitably aliquoted. The kits of the present disclosure will also typically include a means for containing the antibody, antigen, and any other reagent containers in close confinement for commercial sale. Such containers may include injection or blow-molded plastic containers into which the desired vials are retained.

### F. Vaccine and Antigen Quality Control Assays

The present disclosure also contemplates the use of antibodies and antibody fragments as described herein for use in assessing the antigenic integrity of a viral antigen in a sample. Biological medicinal products like vaccines differ from chemical drugs in that they cannot normally be characterized molecularly; antibodies are large molecules of significant complexity and have the capacity to vary widely from preparation to preparation. They are also administered to healthy individuals, including children at the start of their lives, and thus a strong emphasis must be placed on their quality to ensure, to the greatest extent possible, that they are efficacious in preventing or treating life-threatening disease, without themselves causing harm.

The increasing globalization in the production and distribution of vaccines has opened new possibilities to better manage public health concerns but has also raised questions about the equivalence and interchangeability of vaccines procured across a variety of sources. International standardization of starting materials, of production and quality control testing, and the setting of high expectations for regulatory oversight on the way these products are manufactured and used, have thus been the cornerstone for continued success. But it remains a field in constant change, and continuous technical advances in the field offer a promise of developing potent new weapons against the oldest public health threats, as well as new ones-malaria, pandemic influenza, and HIV, to name a few—but also put a great pressure on manufacturers, regulatory authorities, and the wider medical community to ensure that products continue to meet the highest standards of quality attainable.

Thus, one may obtain an antigen or vaccine from any source or at any point during a manufacturing process. The quality control processes may therefore begin with preparing a sample for an immunoassay that identifies binding of an antibody or fragment disclosed herein to a viral antigen. Such immunoassays are disclosed elsewhere in this document, and any of these may be used to assess the structural/antigenic integrity of the antigen. Standards for finding the sample to contain acceptable amounts of antigenically correct and intact antigen may be established by regulatory agencies.

Another important embodiment where antigen integrity is assessed is in determining shelf-life and storage stability. Most medicines, including vaccines, can deteriorate over time. Therefore, it is critical to determine whether, over time, the degree to which an antigen, such as in a vaccine, degrades or destabilizes such that is it no longer antigenic and/or capable of generating an immune response when administered to a subject. Again, standards for finding the sample to contain acceptable amounts of antigenically intact antigen may be established by regulatory agencies.

In certain embodiments, viral antigens may contain more than one protective epitope. In these cases, it may prove useful to employ assays that look at the binding of more than one antibody, such as 2, 3, 4, 5 or even more antibodies. These antibodies bind to closely related epitopes, such that they are adjacent or even overlap each other. On the other hand, they may represent distinct epitopes from disparate parts of the antigen. By examining the integrity of multiple epitopes, a more complete picture of the antigen's overall integrity, and hence ability to generate a protective immune response, may be determined.

Antibodies and fragments thereof as described in the present disclosure may also be used in a kit for monitoring the efficacy of vaccination procedures by detecting the presence of protective EEEV antibodies. Antibodies, antibody fragment, or variants and derivatives thereof, as described in the present disclosure may also be used in a kit for monitoring vaccine manufacture with the desired immunogenicity.

### VI. Examples

The following examples are included to demonstrate preferred embodiments. It should be appreciated by those of skill in the art that the techniques disclosed in the examples that follow represent techniques discovered by the inventor to function well in the practice of embodiments, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the disclosure.

### Example 1—Materials & Methods

Human subject information. EEEV-373 was isolated from one research subject as previously described⁴². Briefly, peripheral blood mononuclear cells (PBMCs) were isolated from peripheral blood samples by density gradient purification and cryopreserved until use. Written informed consent was given from the subjects and protocols were approved by the Institutional Review Board (IRB) at Vanderbilt University Medical Center for the recruitment and collection of blood samples used in this study.

Mouse model. Animal models were performed as previously described^{40,42}. C57BL/6 mice were purchased from Jackson Laboratories. All animal procedures were carried out in accordance with the recommendations in the Guide for the Care and Use of Laboratory Animals of the National Institutes of Health. The protocols were approved by the Institutional Animal Care and Use Committee at the Utah State University IACUC protocol #10025.797

Cell lines. Cell lines were maintained as previously described^{39,40,42}. Routine mycoplasma detection was performed using a universal mycoplasma detection kit (ATCC) and in all cases gave negative results for the cell lines tested except for the kit positive control reagent.

Viruses and virus-like particles (VLPs). The chimeric virus Sindbis virus (SINV; TR339)/Eastern equine encephalitis virus (EEEV; strain FL93-939) was previously described⁴³ Briefly, the structural proteins genes of SINV TR339 were replaced with the structural protein genes of EEEV (FL93-939) under control of the SINV 26S subgenomic promoter in the cDNA clone. For wild-type virus neutralization studies, EEEV (strain FL93-939) was derived from a cDNA clone as previously described^{39,50}. For animal studies, the EEEV FL93-939 strain used was obtained from Dr. Robert Tesh, World Reference Center for Emerging Viruses and Arboviruses (University of Texas Medical Branch), Galveston, TX and passaged twice in Vero cells prior to use in mice^{40,42}. Purified EEEV VLPs were kindly provided by Dr. John Mascola⁵¹.

Recombinant proteins. Recombinant EEEV E2/E1 and monomeric E1 proteins (strain FL93-939) were expressed and purified as previously described, respectively^{39,40}. Briefly, the proteins were codon-optimized, synthesized, and cloned into the mammalian expression vector pcDNA3.1 (+). Recombinant proteins were produced in Expi293F cells using the ExpiFectamine 293 transfection kit according to manufacturer's instructions (Thermo Fisher Scientific). Cell supernatant was clarified and purified through a HisTrap excel column (Cytiva) on an ÄKTA pure 25M chromatography system. Recombinant EEEV E3E2/E2 (strain v105), which contains a mixture of E3E2 and E2 glycoproteins, was purchased from IBT Bioservices.

SINV/EEEV production and purification. For binding and neutralization assays, SINV/EEEV was produced using the methods previously described^{39,42}. For cryo-EM studies, SINV/EEEV was purified according to the previously described protocol^{6,40}. For liposomal fusion inhibition studies, SINV/EEEV was purified and labeled with Vybrant DiD cell-labeling solution as previously described⁴⁰.

Human hybridoma antibody generation and sequence analysis. EEEV-373 hybridoma cell lines secreting EEEV-373 IgG were generated, selected, and purified as previously described^{39,40,42}. Recombinant mAbs were produced using the ExpiCHO expression system (Thermo Fisher Scientific) as previously described⁴². Antibodies were purified from clarified supernatants using HiTrap MabSelect SuRe (Cytiva) columns on an ÄKTA Pure 25M chromatography system and further concentrated using 50K MWCO Amicon® Ultra centrifugal filter units (MilliporeSigma), desalted, and buffer exchanged with 7K MWCO Zeba desalting columns (Thermo Fisher Scientific). For the generation of EEEV-373 Fab or F(ab′2) molecules, hybridoma mAb was cleaved using the IgdE cysteine enzyme FabALACTICA® (Genovis) as previously described⁴² or using the IdeS protease (Promega), respectively. Briefly, IgG was incubated with FabALACTICA® or IdeS protease (1 unit/1 μg of IgG) overnight at room temperature or 37° C. for 30 to 60 minutes, respectively. Fab or F(ab′2) molecules were then purified using the CaptureSelect™ IgG-Fc affinity matrix (Thermo Fisher Scientific). For sequence analysis of EEEV-373, the gene segments and V-region nucleotide percent identity compared to germline were determined using the ImMunoGenetics (IMGT) database as previously described^{39,42}.

Protein EC₅₀ ELISA. To determine the binding EC₅₀ values of EEEV-373 to recombinant antigens an ELISA was performed as previously described^{39,42}. Briefly, EEEV VLPs (2 μg/mL) and recombinant EEEV E3E2/E2 (2 μg/mL), E2/E1 (3.84 μg/mL), or E1 (2 μg/mL) proteins were diluted in 1× D-PBS to coat 384-well ELISA plates (Thermo Fisher Scientific) and incubated at 4° C. overnight. The plates were aspirated and incubated with blocking solution (2% non-fat dry milk (Bio-Rad), 2% goat serum (Thermo Fisher Scientific) in 1× D-PBS-T [1× D-PBS+0.05% Tween 20]) for 1 hour at room temperature. EEEV-373 IgG, F(ab′)₂, or Fab molecules were initially diluted to 33 nM then serially diluted 3-fold in blocking solution (1% non-fat dry milk, 1% goat serum in 1× D-PBS-T). Abs then were added to the plates and incubated at room temperature for 2 hours. The plates then were washed 3× with 1× D-PBS-T and incubated with a solution of secondary antibodies (goat anti-human kappa-HRP and goat-anti-human lambda-HRP [Southern Biotech]) diluted 1:4,000 in blocking solution (1% non-fat dry milk, 1% goat serum in 1× D-PBS-T) for 1 hour at room temperature. The plates then were washed 3× with 1× D-PBS-T followed by the addition of One-Step™ Ultra-TMB ELISA substrate solution (Thermo Fisher Scientific) for ˜5 minutes. The reaction was stopped with 1N HCl and optical density then was read at 450 nm with a BioTek™ plate reader.

Binding kinetics analysis using a quartz crystal microbalance biosensor. EEEV VLPs (35 μg/mL) diluted in 10 mM HEPES buffer (pH 7.4) were immobilized onto LNB carboxyl sensor chips (Attana AB) by amine coupling using EDC (1-ethyl-3-[3-dimethylaminopropyl]-carbodiimide hydrochloride) and Sulfo-NHS (N-hydroxysulfosuccinimide) chemistry on the Attana Cell™ 200 instrument (Attana AB). For binding assays, first to account for background binding, the sensor chips were injected with running buffer (1× HBS-T). EEEV-373 IgG (33, 8, or 0.33 nM), F(ab′)₂ (35, 10, or 0.35 nM), or Fab (35, 10, or 0.35 nM) molecules diluted in running buffer then were injected to observe Ab binding to EEEV VLPs. Between Ab injections, EEEV VLP immobilized LNB carboxyl sensor chips were regenerated using 10 mM glycine pH 3.5 buffer. Assays were performed at a flow rate of 10 μL/min at 22° C. using 50- and 600-seconds association and dissociation times, respectively. Data were collected on the Attester software (Attana AB). A blank LNB carboxyl sensor chip was used as a reference and subtracted using the Evaluation software (Attana AB). Curves were fitted and kinetic parameters calculated using a bivalent (IgG and F(ab′)₂) or 1:1 (Fab) binding model using the TraceDrawer software (Ridgeview Instruments AB).

Binding kinetics analysis using biolayer interferometry. Biolayer interferometry was performed using an Octet® HTX system (Sartorius). Following a baseline measurement in kinetics buffer (Sartorius), 10 μg/mL of EEEV-373 IgG, F(ab′)₂, and negative control IgG molecules, or 6.7 μg/mL of EEEV-373 and EEEV-94 Fab molecules were separately loaded to protein L biosensors (Sartorius). The biosensors were then dipped into kinetics buffer followed by incubation with EEEV VLPs at concentrations of 0, 0.2, 0.5, 0.8, 1, or 2 nM for 300 seconds. Dissociation was then measured by biosensor incubation in kinetics buffer for 1,800 seconds. A reference subtraction was performed for each antibody and curves were fitted using a 1:1 binding model. Kinetic analysis was performed using the Octet® Data Analysis v12.2 software (Sartorius).

Cell surface display EEEV binding. Binding analysis of mAbs to cells expressing alphavirus structural proteins has been previously described^{39,42,52}. Briefly, Expi293F cells were transiently transfected with a plasmid (pcDNA3.1 (+)) containing the structural proteins (capsid-E3-E2-6K-E1) of EEEV (strain FL93-939) using the ExpiFectamine transfection kit according to manufacturer's protocols (Thermo Fisher Scientific). Cells were incubated at 37° C. in a humidified atmosphere of 8% CO₂ for 24 hours. Cells then were harvested, fixed with 1% PFA/PBS, washed twice with 1× D-PBS, and stored at 4° C. in FACS buffer (1× D-PBS, 2% ultra-low IgG FBS, 2 mM EDTA) until use. Cells were plated at 40-50,000 cells/well in 96-well V-bottom plates. EEEV-373, rEEEV-106, or rDENV-2D22 IgG were diluted to 1 μg/mL in FACS buffer and incubated with the cells at 4° C. for 1 hour. Cells then were washed with FACS buffer and incubated with secondary antibodies (anti-human IgG-PE and anti-human IgA-PE [Southern Biotech]) diluted 1:1,000 in FACS buffer for 1 hour at 4° C. Cells then were washed in FACS buffer, and the number of events was collected on an IntelliCyt® iQue Screener Plus flow cytometer (Sartorius).

Dynamic light scattering (DLS). DLS was performed to assess antibody-mediated aggregation of virus particles as previously described⁴⁰. Briefly, EEEV-373 IgG, F(ab′)₂, or Fab molecules were mixed and incubated at 37° C. for 30 minutes with EEEV VLPs at Ab:VLP molar ratios ranging from 1:1 to 1,000:1. VLPs were incubated without mAb to control for particle size (˜70 nm). DLS was then performed at 37° C. using a biologics stability screening platform (Uncle) and Uncle version 5.0 analysis software (Unchained Labs). Mean peak intensity was used to determine the hydrodynamic diameter (nm) of EEEV VLPs.

ApoER2 and VLDLR receptor blockade. EEEV VLPs were diluted to 2 μg/mL in 1× D-PBS to coat 384-well ELISA plates (Thermo Fisher Scientific) and incubated at 4° C. overnight. The plates were aspirated and incubated with blocking solution (2% non-fat dry milk (Bio-Rad), 2% goat serum (Thermo Fisher Scientific) in 1× D-PBS-T [1× D-PBS+0.05% Tween 20]) for 1 hour at room temperature. EEEV-373 or a negative control mAb were diluted for a final concentration of 1 to 20 μg/mL in blocking solution (1% non-fat dry milk, 1% goat scrum in 1× D-PBS-T). MAbs then were added to the plates and incubated at room temperature for 1 hour. Recombinant human apolipoprotein E R2 protein (ApoER2; R&D Systems) or human very low-density lipoprotein receptor (VLDLR; R&D Systems) protein were diluted to a final concentration of 5 μg/mL. ApoER2 or VLDLR were then added to the plates and incubated at room temperature for 1 hour. The plates then were washed 3× with 1× D-PBS-T and incubated with a solution of secondary antibodies (anti-his-HRP [Thermo Fisher Scientific]) diluted 1:2,000 in blocking solution (1% non-fat dry milk, 1% goat serum in 1× D-PBS-T) for 1 hour at room temperature. The plates then were washed 3× with 1× D-PBS-T followed by the addition of One-Step™ Ultra-TMB ELISA substrate solution (Thermo Fisher Scientific) for ˜5 minutes. The reaction was stopped with 1N HCl and optical density then was read at 450 nm with a BioTek™ plate reader. Percent binding of ApoER2 or VLDLR was normalized to the average optical density value for binding in the presence of the negative control mAb.

SINV/EEEV neutralization assays. Neutralization assays, including the focus reduction neutralization test (FRNT) and post-attachment assays, were formed as previously described^{39,40,42}. Briefly, EEEV-373 IgG, F(ab′)₂, or Fab molecules were initially diluted to 33 nM then serially diluted 3-fold in medium (DMEM/2% ultra-low IgG FBS/10 mM HEPES). Abs were then incubated with ˜100 FFU/well of SINV/EEEV and incubated together for 1 hour at 37° C. in a humidified atmosphere of 5% CO₂ or 4° C., respectively. Virus foci were detected as previously described.

Liposomal fusion inhibition assay. Liposomes (2 mM) containing 1,2-dioleoyl-sn-glycero-3-phosphocholine (DOPC), 1,2-diolcoyl-sn-glycero-3-phosphoethanolamine (DOPE), sphingomyelin (chicken egg), and cholesterol (ovine wool) were prepared as previously described⁴⁰. Fusion inhibition was assessed as previously described⁴⁰. Briefly, Vybrant DiD-labeled SINV/EEEV particles were incubated with EEEV-373 or negative control IgG (9 μg), F(ab′)₂ (6.2 μg), or Fab (3 μg) molecules for 30 minutes at room temperature with shaking (300 rpm). Buffer with no Ab or liposome controls were also included. Prepared liposomes (2 mM) then were added, expect for the no liposome control, and loaded onto a SpectraMax iD5 multimode plate reader (Molecular Devices) at 37° C. Sequential injections of 0.1 M MES-0.2 M acetic acid (pH 5.0) were performed using an automated injector and mixed immediately. Fluorescence (excitation at 635 nm, emission at 675 nm) then was read every 10 seconds for a total of 100 seconds.

Egress inhibition assay. Egress inhibition was performed as previously described⁴⁰ Briefly, BHK-21 cells were inoculated with SINV/EEEV (MOI 1) in medium (DMEM/2% ultra-low IgG FBS/10 mM HEPES) and then incubated at 37° C. in a humidified atmosphere of 5% CO₂ for 2 hours. Cells then were washed 6× with medium containing 25 mM NH₄Cl. EEEV-373 or rDENV-2D22 IgG were diluted to 10 μg/mL in the same medium and added to the cells. The cells then were incubated at 37° C. in a humidified atmosphere of 5% CO₂ for up to 6 hours. Supernatant was harvested after 1- and 6-hour incubation periods. Viral RNA then was extracted from the supernatant using the Maxwell® Viral Total Nucleic Acid purification kit on the Maxwell® system (Promega). qRT-PCR and quantification of relative RNA copies/μL were performed as previously described using the SuperScript™ III Platinum One-Step qRT-PCR kit (Thermo Fisher Scientific) 40.

SINV/EEEV escape mutant generation and analysis. To assess the ability for viral escape in the presence of EEEV-373, a real-time cell analysis (RTCA) assay and xCELLigence RTCA Multiple Plates instrument was used. The RTCA assay was performed by adding DMEM/2% ultra-low IgG FBS/10 mM HEPES to each well of a 96-well E-plate to establish a background reading. Vero cells (˜18,000) were then seeded to each well of the E-plate overnight. Continuous monitoring of cell index values was performed every 15 minutes and analyzed using the RTCA software. Initial establishment of the neutralization potency of EEEV-373 in the RTCA assay was performed by incubating SINV/EEEV at an MOI of ˜0.06 with serial three-fold dilutions of EEEV-373 or a negative control mAb starting at 10 g/mL for 1 hour at 37° C. in 5% CO₂. The virus:mAb complexes were then added to the Vero cell monolayers and continuously monitored for changes in cell index values over time. Wells containing only medium or SINV/EEEV were included as controls. Normalized cell index values approximately 48 hours post-inoculation were determined to establish the percent neutralization. To select for escape mutant viruses, a similar experiment was performed with some modifications. SINV/EEEV at an MOI of ˜0.06 was incubated with saturating concentrations (50 and 10 μg/mL) of EEEV-373 or a negative control mAb for 1 hour at 37° C. in 5% CO₂. The virus:mAb complexes were then added to the Vero cell monolayers and continuously monitored for changes in cell index values over time.

Wells in which cytopathic effect was observed in the presence of EEEV-373 compared to control wells were identified as potential escape mutants and supernatant collected. To verify the observed phenotype, supernatants were incubated in the absence or presence of EEEV-373 (20 μg/mL), added to confluent Vero cell monolayers in a 6-well plate, and then incubated at 37° C. in 5% CO₂. Supernatants were then collected, and viral RNA was extracted using a QIAmp viral RNA extraction kit (Qiagen). cDNA corresponding to the E2 protein gene was then generated and amplified using a SuperScript™ III One-Step RT-PCR system with Platinum™ Taq DNA polymerase (Thermo Fisher Scientific) with the following forward and reverse primers, respectively:

(SEQ ID NO: 11)
5′ ATGTGCGTCCTGGCCAATATCACGTTTCC 3′
and
(SEQ ID NO: 12)
5′ GAACAAAACTAGGGCAACCACTGCTGTAGC 3′

The PCR product was then purified using a QIAquick PCR purification kit (Qiagen). The resulting product was sequenced using long-read Oxford Nanopore technology (Plasmidsaurus). From the obtained sequences, variants were identified and compared to consensus sequences or the corresponding input wild-type SINV/EEEV sequence. Escape mutant viruses were identified as variants obtained only in the presence of EEEV-373.

EEEV plaque reduction neutralization test. The neutralization activity of EEEV-373 against wild-type EEEV (strain FL93-939) was performed as previously described39. Briefly, EEEV-373 (75 nM) was serially diluted 2-fold and incubated with ˜100 PFU of EEEV at 37° C. for 1 hour. Anti-EEEV ascites fluid (ATCC) was used as a positive control. Ab:virus complexes then were added to Vero cell monolayer cultures and incubated at 37° C. for 1 hour. An agarose overlay then was added and plates were incubated at 37° C. for 2 days. For detection of plaques, a neutral red overlay was added.

Mouse subcutaneous EEEV challenge model. Mice were challenged with EEEV by bilateral subcutaneous injections as previously described^{40,42}. Mice were treated with EEEV-373 at 10 mg/kg via a single intraperitoneal injection 24 hours after EEEV challenge. Previously published controls, including rDENV-2D22 IgG and normal controls, performed at the same time were included for comparison 39.40. The mice were monitored for 21 days post-virus inoculation (dpi) for survival, disease signs, and body weight. Serum was collected from the mice 3 dpi to assess virus titers via an infectious cell culture assay as previously described^{39,40}. Sample preparation for cryo-EM and data collection. SINV/EEEV particles were mixed with EEEV-373 at a ratio of 1:1 (virus:IgG) and 3 μL of the virus-IgG mixture was immediately loaded onto glow discharged lacey carbon grids in a BSL2-containment facility. The grids then were blotted for 3.2 seconds and plunge frozen in liquid ethane using a Gatan CP3. The entire process was performed under 30 seconds to avoid potential aggregation of the particles. The frozen grids of the complexes were imaged using a Gatan Bioquantum-K3 camera mounted on a Titan Krios (Thermo Fisher Scientific) microscope. The data collection parameters are listed in Table 1.

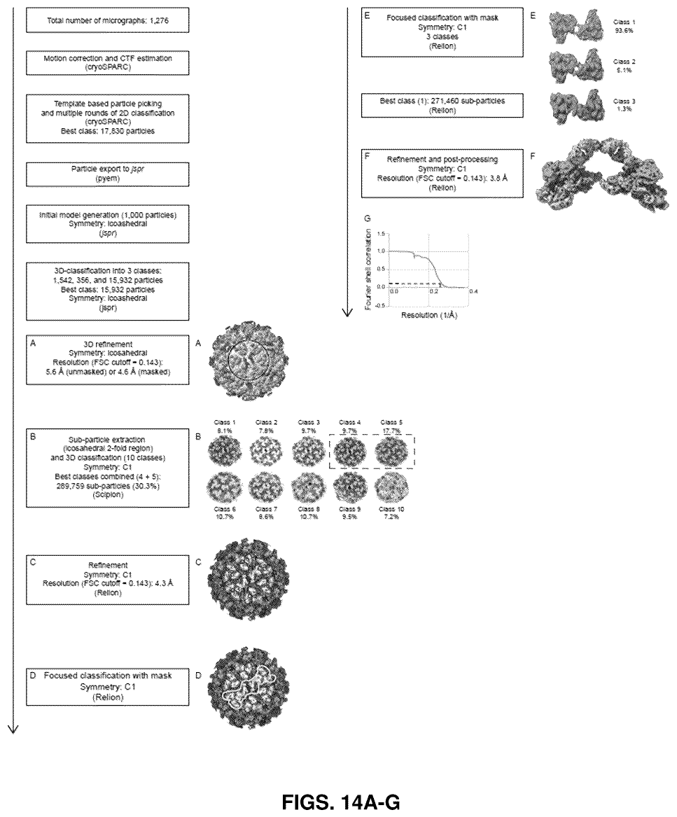

Cryo-EM data processing. Data was processed as previously described⁴⁰. The resolution of the final map (4.6 Å) was calculated using the Gold Standard Fourier Shell Correlation (GSFSC) coefficient at 0.14353. Although the quality of the map was sufficient to resolve the glycoproteins, the density of the bound IgG molecule was quite poor. To improve the density of the bound IgG, localized reconstruction of the i2 was performed. For this, sub-particles at the i2 were extracted and 3D classified into 10 classes without imposing any symmetry (C1) in Scipion⁵⁴. The best classes with well-defined structural features were pooled (289,759 sub-particles) and further refined in Relion⁵⁵ with C1 symmetry until the refinement converged. At this step, the estimated resolution according to GSFSC coefficient at 0.143 was 4.3 Å. Although the quality of the map for the bound IgG improved in comparison to the icosahedral map, it was still not useful for atomic model fitting. Therefore, a mask was applied to the two IgG bound q3 spikes and another round of 3D classification within the masked volume into three classes were carried out in Relion. One class contained with well-defined features and this class (271,460 sub-particles) was further refined using C1 symmetry in Relion. This generated a map of the bound IgG of sufficient quality to begin atomic model fitting. No density was observed for either the hinge or Fc region of the antibody. The resolution of this final map was estimated to be 3.8 Å according to the GSFSC coefficient at 0.143. Although the resolution estimation of the map is global, visual inspection revealed a resolution range across the map. Therefore, local resolution estimation of the map was carried out in Relion, which estimated the resolution of the epitope-paratope interaction surface to be around 5 Å. The map was sharpened using Autosharpen in Phenix⁵⁶. The statistics of the data collection and structure reconstruction are listed in Table A. A schematic of the data processing is described in FIGS. 14A-G.

Model building and fitting into the cryo-EM density map. The ectodomain models of EEEV E2 and E1 were obtained from the Protein Data Bank (PDB ID: 6MX4). A homology model of EEEV-373 was built using AlphaFold 3. Rigid body fitting of the ectodomains and the Fab arms of the IgG into the 3.8 Å map corresponding to the two q3 spikes cross-linked by EEEV-373 were carried out in ChimeraX⁵⁷. The fitted models then were further refined against the map using the Real Space Refinement routine in Phenix56. For the E2 and E1 ectodomains simulated annealing was included in the refinement protocol and the variable domains of the IgG were refined without simulated annealing. The resolution of the map corresponding to the constant domains were significantly worse than the rest of the map. Therefore, the constant domains were fitted as rigid bodies. The quality of the model geometry and the clash score were iteratively improved by a combination of Isolde 58, Coot 59 and real_space_refinement routine in Phenix. The viral surface area buried by EEEV-373 was calculated using ChimeraX. For fitting into the icosahedral map, the refined model of one of the q3 spikes in complex with the Fab arm of the IgG and a model of the ectodomain of the i3 E2/E1 heterodimer (PDB ID: 6MX4) were fitted in UCSF ChimeraX following T=4 quasi-symmetry of the asymmetric unit as rigid bodies. Then, the asymmetric unit around the fitted model was extracted using the phenix.map_box command. Only the i3 arm of the model was subjected to the Real_Space_Refine routine in Phenix using default settings against the extracted map corresponding to the asymmetric unit. The model quality was improved as described above. Finally, the refined model is again fitted as a rigid body in the icosahedral map in ChimeraX following T=4 quasi-symmetry.

### Example 2—Results

Identification and characterization of the potently neutralizing and efficacious human mAb EEEV-373. The inventors isolated the human mAb EEEV-373 (IgG1, κ) from the B cells of an individual with prior documented natural EEEV infection using human B cell hybridoma cell technology. EEEV-373 was selected based on binding reactivity in ELISA to the chimeric virus Sindbis (SINV)/EEEV, EEEV virus-like particles (VLPs), and recombinant EEEV heterodimer (E2/E1) protein. The chimeric virus SINV/EEEV encodes the nonstructural proteins of SINV and the structural proteins of EEEV, which enables safe use of virus particles in biosafety level (BSL)-2 conditions⁴³.

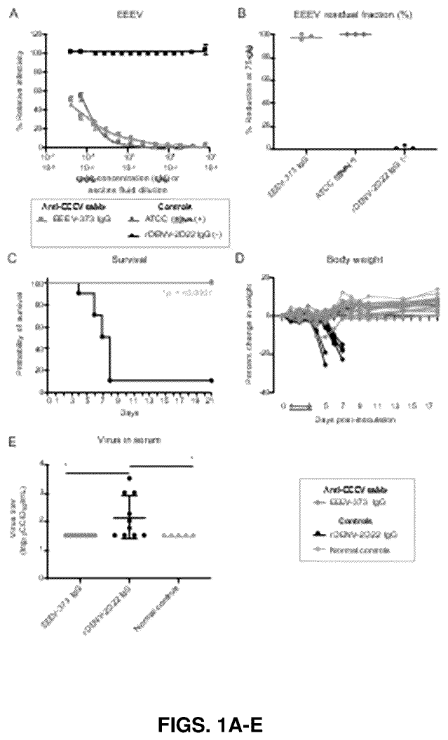

To further characterize EEEV-373, the inventors assessed the neutralization activity of hybridoma-cell-derived EEEV-373 IgG against wild-type EEEV (strain FL93-939) under BSL-3 conditions through a plaque reduction neutralization assay (FIG. 1A). EEEV-373 exhibited potent neutralization activity against EEEV, with a half-maximal inhibitory concentration (IC₅₀) value of <37 pM (11 ng/mL). A minor residual fraction of non-neutralized virus (2%) was present at the highest concentration tested of 75 nM (22.5 μg/mL), indicating incomplete neutralization of EEEV (FIG. 1B).

Next, the inventors assessed the in vivo efficacy of EEEV-373 IgG against EEEV (strain FL93-939) infection in a subcutaneous (s.c.) treatment challenge model (FIGS. 1C-1E). In this model, EEEV was inoculated s.c. (10^{3.3} CCID₅₀) and antibody (IgG) was administered intraperitonially (i.p.) 24 hours after virus inoculation. Mice were followed for a total of 21 days for survival, and viremia was assessed 3 days after virus inoculation using a plaque assay. In the EEEV-373 treatment group, 100% of the animals survived, compared to a 10% survival rate in the negative control group treated with the recombinantly-derived dengue virus-specific mAb rDENV-2D22 (IgG1, λ)⁴⁴ (FIG. 1C). The body weight of the animals corresponded with survival, in which body weight was relatively stable for the EEEV-373 treatment group compared to an observed loss in body weight for the rDENV-2D22 treatment group (FIG. 1D). The inventors also observed a significant reduction in virus titer in the serum to the limit of detection of the assay for animals treated with EEEV-373, compared to those that received rDENV-2D22 (FIG. 1E).

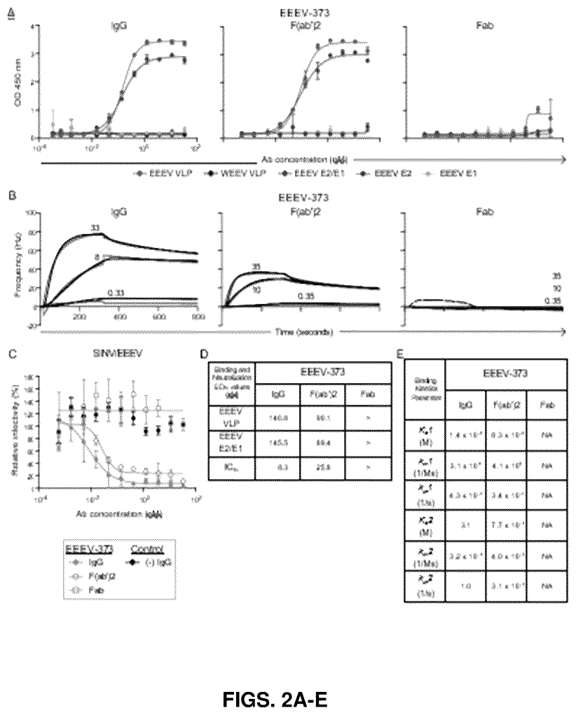

EEEV-373 requires bivalent interactions for binding and neutralization of SINV/EEEV. To further characterize EEEV-373, the inventors addressed whether bivalency is required for binding in ELISA (FIG. 2A). EEEV-373 as cither bivalent IgG or F(ab′)₂ molecules bound to EEEV VLPs and recombinant EEEV E2/E1 protein. However, binding to recombinant monomeric EEEV E2 or E1 protein was not observed at the concentrations tested, which suggests EEEV-373 preferentially binds to a complex quaternary epitope. Maximal optical density signal was observed for binding to EEEV VLPs compared to EEEV E2/E1 protein, which suggested that there may be an avidity effect conferred by bivalent binding to virus particles. In contrast, EEEV-373 tested as monovalent Fab molecules did not appreciably bind to EEEV VLPs, recombinant EEEV E2/E1, E2, or E1 proteins at the concentrations tested. The lack of binding observed indicates EEEV-373 requires bivalent interactions for stable binding. To further assess the dependence of EEEV-373 on bivalency, the inventors tested the neutralization potency of EEEV-373 against SINV/EEEV (FIG. 2C). Consistent with the binding profile of EEEV-373, the IgG and F(ab′)₂ formats of the antibody neutralized SINV/EEEV with similar potencies, although a ˜3-fold reduction in IC₅₀ value was observed for the F(ab′)₂ format (FIG. 2D). In contrast, EEEV-373 Fab molecules failed to neutralize SINV/EEEV.

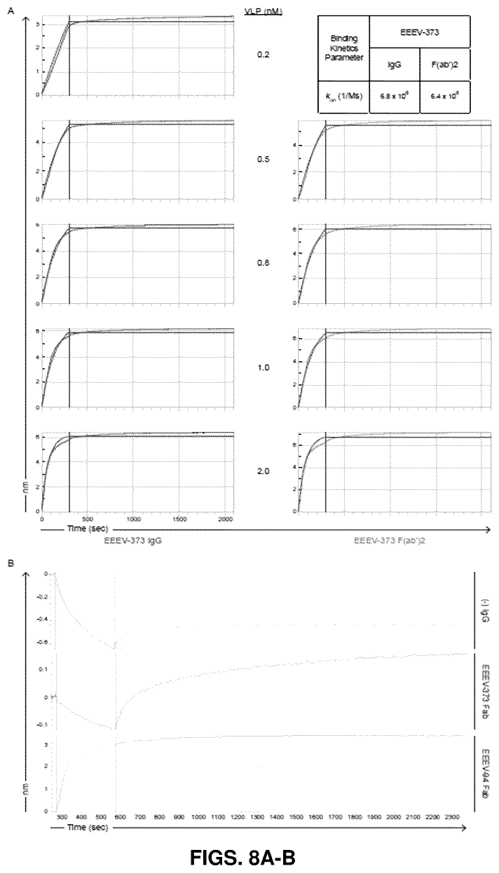

The inventors next assessed the binding kinetics of EEEV-373 IgG, F(ab′)₂, or Fab molecules with EEEV VLPs using quartz crystal microbalance biosensors (FIG. 2B and FIG. 2E). In this assay, they also observed binding of EEEV-373 to EEEV VLPs in a bivalent manner. For binding of EEEV-373 IgG or F(ab′)₂ molecules to EEEV VLPs, they observed a biphasic association pattern in which there was a fast initial binding interaction (105 Ms-1) followed by a slower second phase interaction (10⁻¹ Ms⁻¹). The KD, kon, and koff values for both IgG and F(ab′)₂ molecules are similar (FIG. 2E), suggesting that the Fc region of the antibody does not play a major role in the binding interaction. Further supporting this, the inventors also observed a similar association profile for binding of bivalent EEEV-373 IgG and F(ab′)₂ molecules to EEEV VLPs using biolayer interferometry (FIGS. 8A-B). In contrast to bivalent molecules, a weak association was observed for Fab molecule binding to EEEV VLPs. This weak association corresponds with the variable detectable signal observed at the highest concentrations of Fab molecules for binding to EEEV VLPs as tested via ELISA (FIG. 2A). However, in comparison, binding of EEEV-373 Fab molecules to EEEV VLPs was not detected using biolayer interferometry (FIGS. 8A-B). In this case, the interaction was comparable to that of a negative control IgG. A potential explanation for the observed kinetic behavior of Fab molecules may be the inability to form stable antibody-antigen complexes. The biphasic association pattern of bivalent IgG or F(ab′)₂ molecules indicates that complex formation involves more than one step. The initial interaction may be energetically weak, as reflected by the poor binding of Fab molecules. The avidity effects of bivalent molecules may compensate for this weak interaction by engagement of two binding sites, shifting the equilibrium towards formation of a high affinity complex.

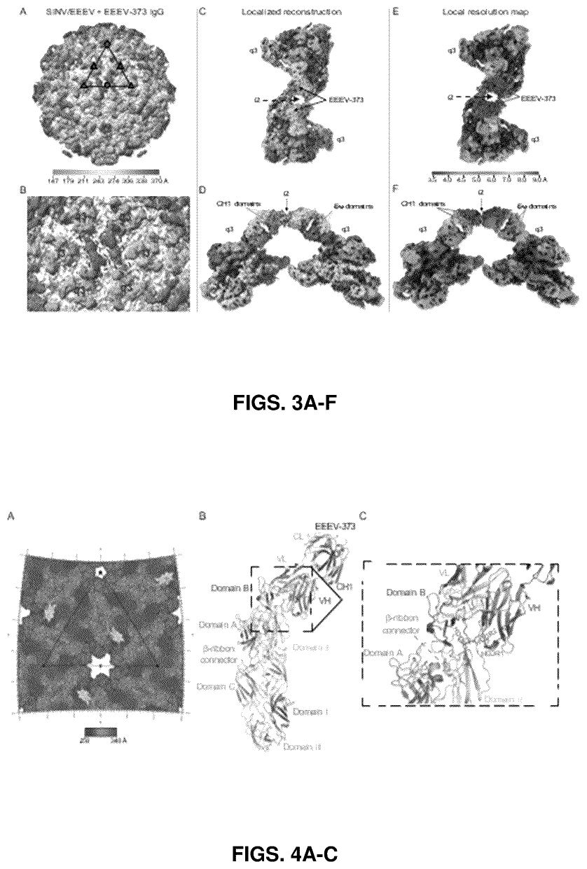

Cryo-EM reconstruction of EEEV-373 as an IgG1 molecule in complex with SINV/EEEV particles. To elucidate the structural determinants of bivalent binding and neutralization, the inventors obtained a three-dimensional cryo-EM reconstruction of SINV/EEEV particles in complex with EEEV-373 as an IgG1 molecule. The reconstruction was determined to ˜4.6 Å with imposed icosahedral symmetry (FIG. 3A). Although the density of the IgG was weak compared to the glycoprotein densities, a single EEEV-373 IgG molecule connecting two q3 spikes diagonally across the i2 axis was visible (FIG. 3A and FIG. 3B). An even weaker and broken density around the i5 axis was also observed. This density likely reflects low IgG occupancy around the i5 axis, suggesting that the predominant mode of binding cross-links the q3 spikes across the i2 axis (FIG. 13D). To improve the density of the antibody and fit atomic models, localized reconstruction combined with focused classification and refinement of the two q3 spikes bound to EEEV-373 were performed, providing a global resolution estimate of ˜3.8 Å (FIG. 3C, FIG. 3D, and FIGS. 14A-G). The overall density of the EEEV-373 IgG molecule at the i2 axis improved, with the local resolution map estimation showing the variable domains resolved significantly better than the constant domains (FIG. 3E and FIG. 3F). The local resolution map estimates the resolution of the variable domains to be ˜5 Å, whereas that of the constant domains was greater than ˜9 Å. The inventors did not observe any density for the hinge or Fc region of the IgG molecule.

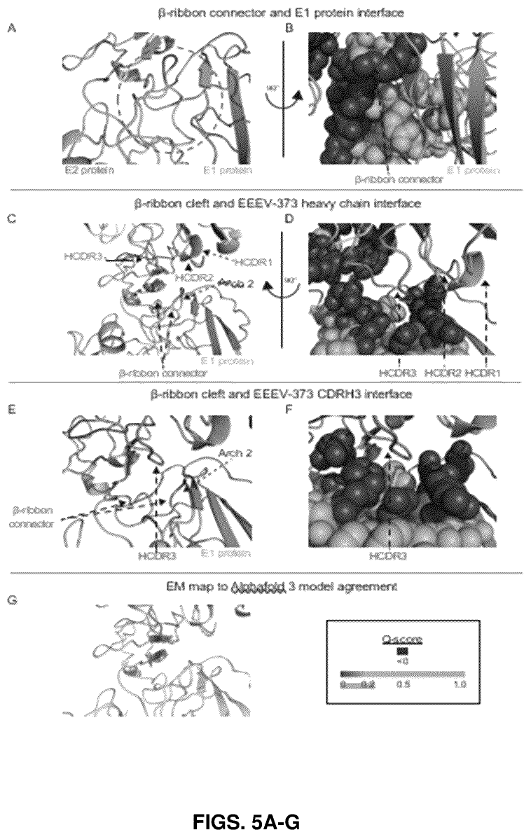

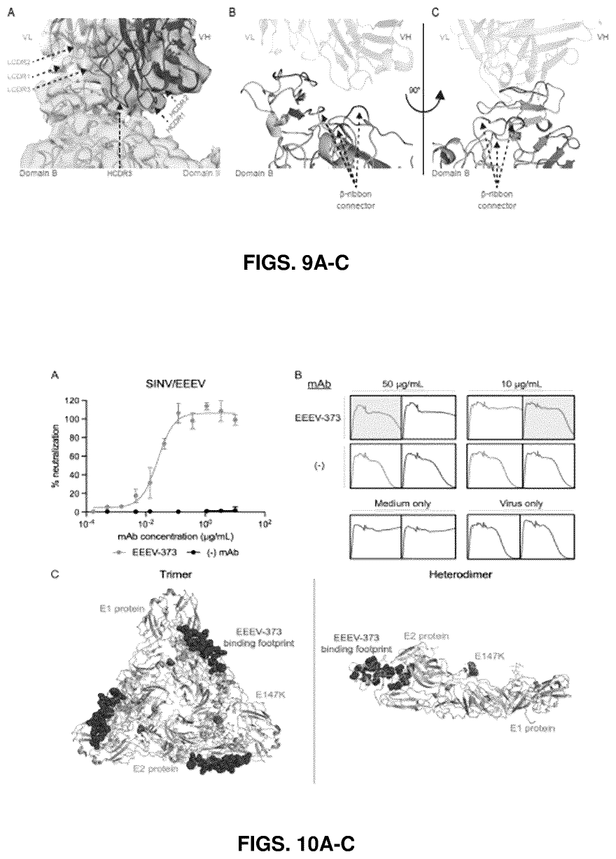

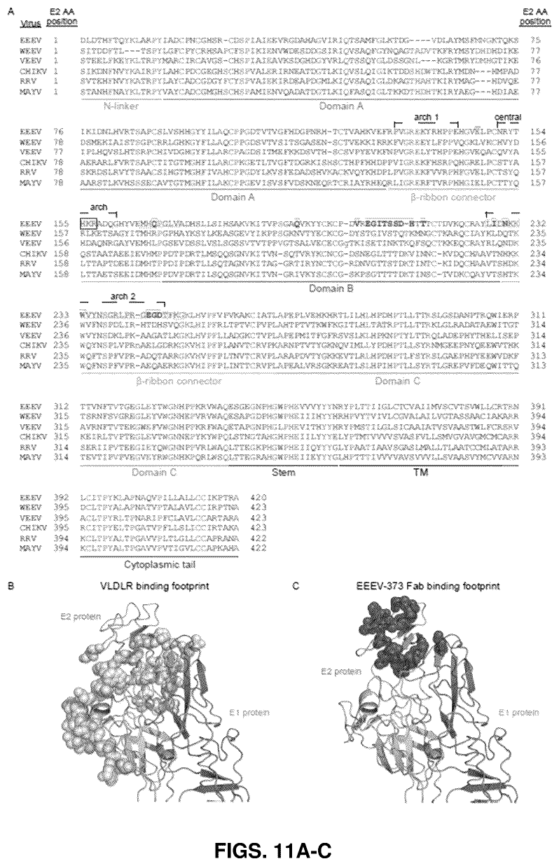

Identification of a quaternary epitope targeted by EEEV-373 on the E2/E1 glycoproteins. From the cryo-EM reconstruction, the inventors observed that EEEV-373 primarily binds the E2 glycoprotein of SINV/EEEV particles (FIGS. 4A-C and FIGS. 9A-C). The total area buried by EEEV-373 on the E2/E1 glycoprotein is 750.5 Å², with the heavy or light chain burying 402 Å² or 348.5 Å², respectively. To determine the binding footprint of EEEV-373 on the E2/E1 glycoproteins, residues within 6 Å of the backbones of the fitted atomic model were identified. The binding footprint of EEEV-373 constitutes several residues on the E2 glycoprotein and one residue (K61) within domain II of the E1 glycoprotein. Except for K61, all remaining residues are not conserved amongst other alphaviruses (FIGS. 11A-C). This finding supports the specificity of EEEV-373 to EEEV, as the inventors did not observe detectable binding to Western equine encephalitis virus (WEEV) VLPs by ELISA at the concentrations tested (FIG. 2A). Based on the binding footprint, the light chain of EEEV-373 binds surface exposed residues within domain B of the E2 glycoprotein, and the heavy chain binds the E2 β-ribbon connector and residue K61 of the E1 glycoprotein (FIG. 4C, FIGS. 5A-G, and FIGS. 9A-C). The heavy chain complementarity-determining regions (CDRs) clamp onto the β-ribbon connector (residues 230 to 248), which is spatially adjacent to domain B and includes the ‘arch 2’ region²² (FIGS. 4A-C, FIGS. 5A-G, and FIGS. 9A-C). The β-ribbon connector essentially is a long linker connecting domains A to B and B to C, with little secondary structure. Natively, the ‘arch 2’ region of the β-ribbon connector forms a narrow cleft-like structure and makes significant interactions with the underlying E1 protein (FIG. 5A and FIG. 5B). Thus, it is conceivable that in the absence of the E1 protein, the β-ribbon connector might be flexible, and as a result the cleft of the ‘arch 2’ region would become unstable. EEEV-373 binding shows that CDRH1 and CDRH2 clamp onto the ‘arch 2’ region (FIG. 5C and FIG. 5D), with CDRH3 inserting into the cleft (FIG. 5E and FIG. 5F). The heavy chain interaction with residue K61 of the E1 glycoprotein is largely due to its long side chain, as no other residue in the vicinity of K61 contributes to the binding footprint of EEEV-373. The interactions observed for the heavy chain CDRs with the E2 glycoprotein, suggest that EEEV-373 binding critically depends on the conformational nature of the heterodimer through the interaction of the E2 β-ribbon connector with the E1 protein, consistent with the ELISA binding data (FIGS. 2A-E).

To further determine the most functionally relevant interactions in the binding footprint of EEEV-373, the inventors assessed the ability for SINV/EEEV to escape neutralization under antibody selective pressure. In the presence of EEEV-373 at saturating antibody concentrations, they identified escape mutant viruses with a E147K mutation in the E2 protein (FIGS. 10A-C). This residue corresponds to the β-ribbon connector outside of the EEEV-373 binding footprint as determined by cryo-EM (FIGS. 11A-C). Given the potential flexibility and conformational dependence of the β-ribbon connector for binding, the E147K mutation may allosterically alter the conformation of the epitope, ablating the neutralization capabilities of EEEV-373.

EEEV-373 cross-links viral q3 spikes using a preferred binding mode. In the cryo-EM reconstruction of EEEV-373 IgG in complex with SINV/EEEV particles, the inventors predominantly observe one binding orientation with the IgG molecule connecting two q3-spikes across the i2 axis (q3.1-q3.3). Based on previous studies, the distance between the C_{H1} domains (Cys216 [Kabat numbering]) of the arms of an IgG molecule is ˜50 Å, allowing for intra-virion cross-linking of icosahedral viruses^{40,45}. However, since the resolution of the constant domains is ˜9 Å in the inventors' reconstruction, the inventors are unable to accurately determine the position of Cys216, which corresponds to Cys228 for EEEV-373. Instead, they used the last residue on strand G of the C_{H1} domain⁶⁰ (Lys222 in EEEV-373) to determine the distances more accurately between the two arms of the EEEV-373. There are 12 residues that act as a long flexible linker between the last residue on strand G and the cysteine residue that forms the first disulfide bond in the hinge region (Cys222 [Kabat numbering]). The length of this flexible segment when fully stretched is ˜48 Å. Thus, the distance criteria for the arms of an IgG molecule ranges from ˜50 to 100 Å.

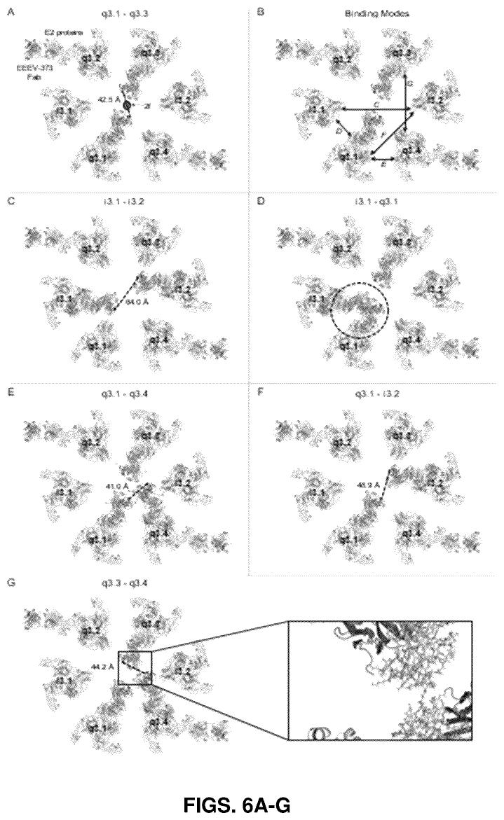

In the preferred q3.1-q3.3 binding mode of EEEV-373, the distance between Lys222 of the CH1 domains is ˜42.5 Å (FIG. 6A), which is well within the range (˜50-100 Å) that allows for an IgG molecule to bind two epitopes^{40,45}. Since the i2 axis of alphaviruses is quasi 6-fold, there are five additional ways EEEV-373 could cross-link viral spikes across the i2 axis (FIG. 6B). To investigate why the inventors observed this preferential q3.1-q3.3 mode of binding, they superimposed the atomic model of the E2 glycoprotein bound to an EEEV-373 Fab arm from the q3.1 spike onto the i3 (i3.1 and i3.2) and q3.4 spikes. This fitting shows that in the i3.1-i3.2 binding mode, the distance between Lys222 of the CH1 domains of EEEV-373 is ˜64 Å (FIG. 6C), which may be too distant or require stretching for both arms of the EEEV-373 IgG molecule to simultaneously bind. Additional binding modes lead to severe clashes of the CL domains, such as in the case of cross-linking adjacent i3.1-q3.1 spikes (FIG. 6D) or between the q3.3-q3.4 spikes (˜44.2 Å; FIG. 6G). These clashes probably restrain binding of the IgG molecule to the preferred q3.1-q3.3 orientation. Interestingly, the q3.1-q3.4 binding mode satisfies both the distance criteria (˜41 Å) and the fitted models do not appear to clash (FIG. 6E). However, the poor resolutions of the CL and the CH1 domains, as estimated from the local resolution maps, suggest these regions are highly flexible. Due to flexibility of the elbow region, movement of the constant domains perpendicular to the pseudo-2-fold axis of the Fab (between the heavy and light chains) can occur³⁶. Therefore, movement of the constant domains could lead to clashes between the two arms of the IgG in the q3.1-q3.4 binding mode. In the observed q3.1-q3.3 binding mode, this movement does not lead to clashes between the two arms of the IgG molecule. The last binding mode (q3.1-i3.2) satisfies the distance criteria (˜48.9 Å) and does not sterically clash (FIG. 6F), yet the inventors do not observe this mode of binding in the structure. The only difference between q3.1-93.3 and q3.1-i3.2 binding modes is the rotation of one Fab arm about 60° with respect to the other arm, which may cause tension on the IgG molecule. However, it is not obvious from the structure as to why this mode of binding is not observed. Together, these observations suggest stringent criteria (i.e., distance and angle of binding) are required for EEEV-373 IgG to bind and form intra-virion spike cross-links.

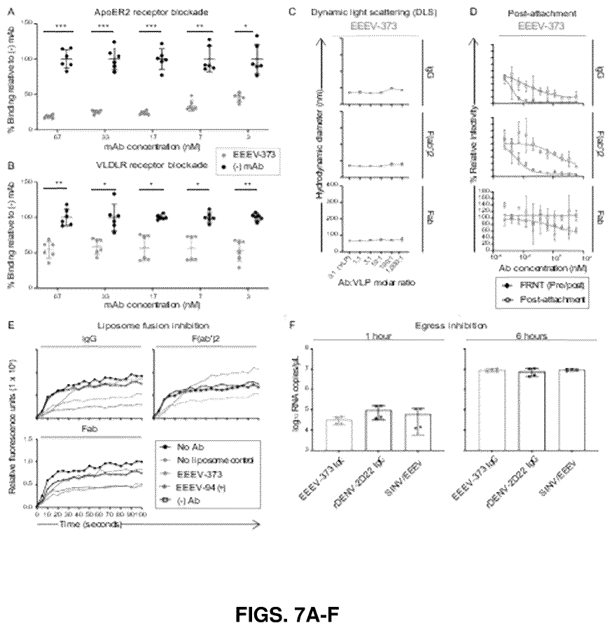

EEEV-373 does not aggregate EEEV VLPs by formation of intra-virion spike cross-links. To functionality validate the observed structural basis of neutralization by EEEV-373, the inventors aimed to elucidate the molecular mechanism of action of EEEV-373 against EEEV. Based on the cryo-EM reconstruction, the observed binding orientation of EEEV-373 displays formation of intra-virion spike cross-links by the IgG molecule. To further corroborate this, they tested the ability of EEEV-373 to aggregate VLPs using dynamic light scattering (DLS) (FIG. 7C). The hydrodynamic diameter of antibody-antigen complexes at different molar ratios can be estimated using DLS. The diameter of the VLP alone was ˜70 nm, which is close to the expected value for an alphavirus particle 16. At any of the molar ratios tested, EEEV-373 as IgG or F(ab′)₂ molecules minimally aggregated VLPs. This finding strongly supports the formation of intra-virion spike cross-links by EEEV-373 binding to two q3 spikes across the i2 axis. At very high molar ratios (>1:100 IgG:VLP), a slight increase in hydrodynamic diameter to ˜100 nm is observed. This finding may result from partial binding of EEEV-373 to additional exposed epitopes present on two neighboring virus particles (inter-virion spike cross-links) following complete occupancy of the preferred binding orientation within the same virus particle (intra-virion spike cross-links). Binding to these exposed epitopes may account for the observed weak density around the i5 axis in the icosahedral reconstruction. As expected, the inventors did not detect aggregation of EEEV VLPs by EEEV-373 Fab molecules.

EEEV-373 blocks receptor binding to EEEV VLPs. Members of the low-density lipoprotein receptor (LDLR) family, apolipoprotein E receptor 2 (ApoER2)²⁸ and very low-density lipoprotein receptor (VLDLR)^{28,29,31}, were recently identified as receptors for EEEV. To assess the ability of EEEV-373 to block receptor interactions, the inventors performed a competition-binding ELISA with ApoER2 and VLDLR (FIG. 7A and FIG. 7B). In this assay, EEEV VLPs were incubated with EEEV-373 or a negative control mAb as IgG1 molecules. ApoER2 or VLDLR then was added to detect whether receptor binding can occur in the presence of mAb. Based on the results, EEEV-373 blocked ˜80% or ˜50% of the interactions with ApoER2 or VLDLR, respectively, compared to the negative control mAb. Recently, the VLDLR binding footprint on the E2 glycoprotein of EEEV was identified^{29,31}. The E2 residues making the most significant interactions with VLDLR (H155, K156, and R157) are on the ‘central arch’ region of the β-ribbon connector, which is adjacent to domain A. This region is distinct from the EEEV-373 binding site, which is on ‘arch 2’ of the β-ribbon connector. Therefore, it is likely that the partial inhibition of VLDLR binding to EEEV by EEEV-373 is through steric blockade of cognate receptor binding or by allosteric modulation of the β-ribbon connector. The receptor binding site for ApoER2 on EEEV is not known yet. However, the results observed suggests EEEV-373 may bind within or nearby the receptor binding site to block or sterically hinder ApoER2 binding (FIGS. 11A-C). As EEEV-373 binds in a preferential manner across the i2 axis, incomplete occupancy of exposed receptor binding sites may occur. Under saturating conditions, 60 out of 80 trimeric spikes are stably occupied with EEEV-373 IgG per virion. For the occupied 60 q3 spikes, only a third of the E2 glycoproteins are bound by IgG, exposing the remaining E2 glycoproteins for receptor binding. This mechanism likely accounts for the observed residual binding of these receptors to EEEV VLPs in the presence of saturating concentrations of EEEV-373.

EEEV-373 partially inhibits SINV/EEEV after virus attachment to cells. The inventors next tested the ability of EEEV-373 to neutralize SINV/EEEV after initial attachment to a Vero cell monolayer using a post-attachment inhibition assay (FIG. 7D), as previously described^{39,40} In this assay, Vero cells were incubated with SINV/EEEV particles at 4° C. to allow for virus attachment. EEEV-373 then was added, and the cells were shifted to 37° C. to enable virus entry. In comparison to a standard focus reduction neutralization assay (FRNT, FIG. 7D), EEEV-373 still neutralized SINV/EEEV as IgG or F(ab′)₂ molecules after virus attachment to Vero cell monolayer cultures. However, a reduced potency in neutralization was observed (˜8- to >200-fold shift in IC₅₀ values for IgG or F(ab′)₂, respectively). This finding may be due to the inaccessibility of all epitopes through interactions between virus particles and cellular receptor or attachment factors on cells, leading to incomplete neutralization. Thus, EEEV-373 may partially neutralize through receptor blocking as previously described and is unable to neutralize as efficiently once the virus has already interacted with cells. Consistent with previous data (FIG. 2C), EEEV-373 Fab molecules did not neutralize SINV/EEEV.

EEEV-373 inhibits initial low-pH-induced fusion of SINV/EEEV with liposomes. The inventors next assessed whether EEEV-373 inhibits low-pH-induced fusion of SINV/EEEV with liposomes as previously described⁴⁰. This assay serves as a surrogate for virus fusion with the endosomal membrane during alphaviral infection of cells by measuring the dequenching of DiD (1, l′-dioctadecyl-3,3,3′,3′-tetramethylindodicarbocyanine, 4-chlorobenzenesulfonate salt)-labeled SINV/EEEV particles. In this assay, DiD-labeled SINV/EEEV particles were incubated with an excess of unlabeled liposomes (200 nm in size) and membrane fusion was triggered by acidification with a low-pH buffer (final pH ˜5.2). Viral fusion was measured by dilution of DiD within the liposomal membrane, and a subsequent increase in fluorescence intensity over time. Inhibition of SINV/EEEV fusion was measured by the reduction of DiD fluorescence dequenching. Pre-incubation of DiD-labeled SINV/EEEV particles with EEEV-373 IgG or F(ab′)₂ molecules led to an initial reduction in relative fluorescence intensity during the first 10-30 seconds after acidification compared to EEEV-373 Fab molecules or negative controls (i.e., irrelevant controls or absence of antibody) (FIG. 7E). This finding suggests bivalent EEEV-373 molecules inhibit initial fusion of SINV/EEEV particles with liposomes or delay fusion kinetics. As time progressed, however, relative fluorescence levels were comparable to those of the negative controls. The reason for this finding is not entirely known. The inability to completely abolish fusion even at saturating concentrations may occur due to incomplete occupancy of necessary epitopes on the virion to block fusion, which enables the virus to compensate for partial occupancy or EEEV-373 binding and subsequently to fuse. Alternatively, multiple mechanisms of action may be at play for the potent neutralization of SINV/EEEV observed by EEEV-373.

EEEV-373 fails to inhibit SINV/EEEV egress. Lastly, the inventors assessed the ability of EEEV-373 to inhibit egress of SINV/EEEV from infected cells, using an established egress assay⁴⁰. Briefly, BHK-21 cell culture monolayers were inoculated with SINV/EEEV at an MOI of 1 for 2 hours at 37° C. Cells were washed extensively to remove any unbound virus and then incubated with EEEV-373 or the negative control mAb rDENV-2D22 as IgG molecules in the presence of ammonium chloride to prevent de novo infection. Supernatant was harvested following 1 or 6 hours to isolate and detect newly made viral RNA molecules by qRT-PCR. Compared with the negative control, EEEV-373 did not significantly inhibit SINV/EEEV egress from BHK-21 cells (FIG. 7F).

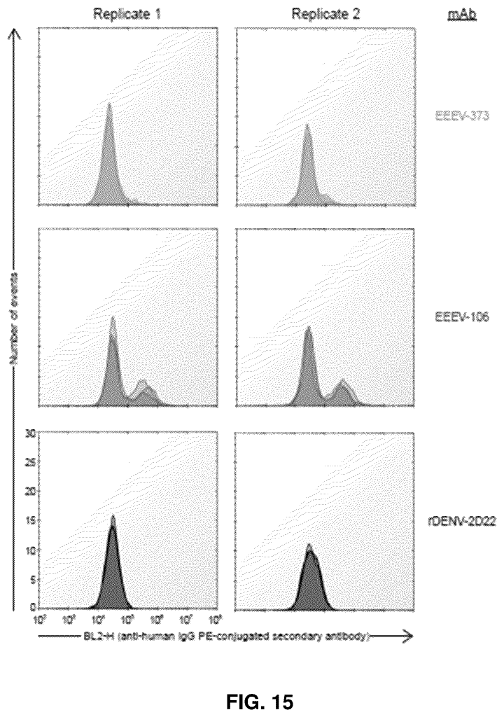

During the alphavirus replication cycle, viral intermediates displaying heterodimers of E2 and E1 are formed on the surface of infected cells prior to egress⁴⁶. Given the observed preferential binding mode of EEEV-373 as IgG molecules to SINV/EEEV particles and lack of detectable inhibition of viral egress, the inventors hypothesized that EEEV-373 may not bind to the intermediate forms of EEEV during viral infection. To test this model, they transiently transfected cells with the structural polyprotein (capsid-E3-E2-6K-E1) of EEEV strain FL93-939 and assessed binding of EEEV-373 IgG molecules (FIG. 15). Compared to the positive control mAb EEEV-06^{39,40}, the inventors observed minimal binding of EEEV-373 to the EEEV structural proteins expressed on the cell surface. This observation supports the hypothesis that EEEV-373 binds to a preferred conformation present principally on intact virions.

### Example 3—Discussion

In this study, the inventors present the structural and molecular basis of epitope recognition and mechanism of action of the potently neutralizing and protective anti-EEEV human mAb EEEV-373. Several aspects of this interaction are interesting from the perspective of therapeutic antibody development and structure-guided immunogen design for vaccine development. First, the inventors present a cryo-EM structure of SINV/EEEV particles in complex with an intact IgG1 molecule. This work adds to a growing but a small list of IgG1-antigen complex structures^{8,9}. Structures of virus-antibody complexes reveal features of interactions that are not always obvious from the corresponding structures of Fab molecules. For example, the bivalent nature and flexibility of the two arms of the antibody can lead to preferred engagement of sites on the viral surface. Monovalent Fab molecules may or may not recapitulate this preferred recognition aspect of the interaction, and thus these studies may lead to inaccurate representation of Fab occupied viral epitopes. As the inventors see in this reconstruction, EEEV-373 preferentially cross-links the two q3-spikes across the i2 axis.

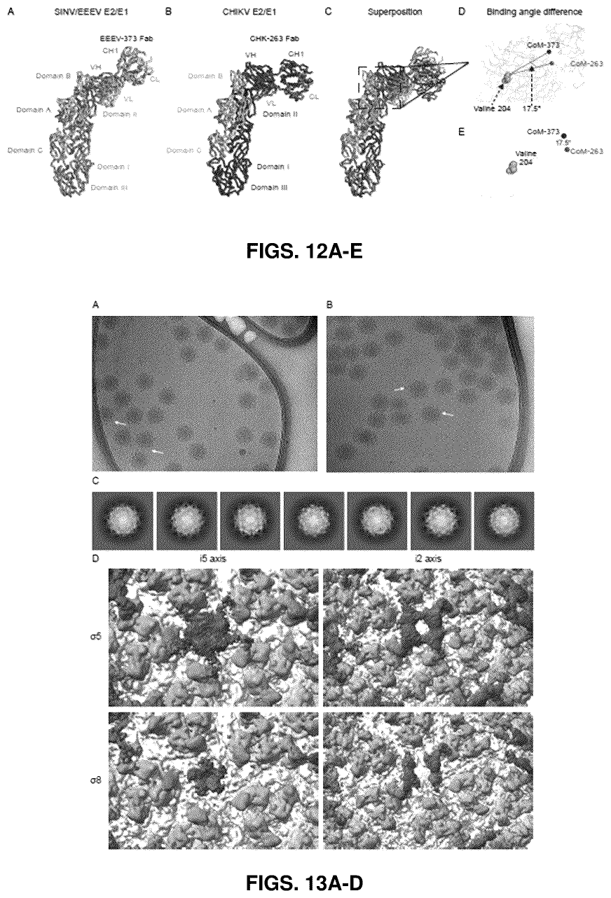

The quaternary epitope revealed by the reconstruction presented here is unique in that the CDRs of EEEV-373 primarily contact residues in domain B and the β-ribbon connector of the E2 glycoprotein. The observed heavy chain CDRH2 interaction with residue K61 of the E1 glycoprotein is largely due to its long side chain, as no other surrounding E1 residues contribute to the binding footprint. This binding mode differs from that reported for the murine anti-chikungunya virus (CHIKV) mAb CHK-263 IgG, which binds to a similar area on CHIKV particles⁹. At the i2 axis, two CHK-263 IgG molecules engage the four q3 spikes. However, due to the low resolution of the reconstructions, the connectivity between the Fab arms of the two IgGs could not be ascertained. The binding modes of CHK-263 IgG suggested by authors correspond to the q3.1-q3.4 and q3.3-q3.4 alternative binding modes depicted here for EEEV-373 (FIGS. 6A-G). Like EEEV-373, weak density of CHK-263 around the i5 axis was also observed, which the authors interpreted as a flexible interaction. In contrast to EEEV-373, CHK-263 tilts ˜17.5° towards and makes contacts with the E1 glycoprotein (FIGS. 12A-E). This difference in the angle of approach may be one of the reasons one EEEV-373 IgG molecule binds diagonally across the i2 axis on SINV/EEEV particles, whereas two CHK-263 IgG molecules are able to bind CHIKV particles. The heavy and light chains of CHK-263 are also flipped with respect to the corresponding chains of EEEV-373. Furthermore, CHK-263 binds as monovalent Fab molecules, which may occur due to recognition of a stabilizing quaternary epitope comprising both proteins of the E2/E1 heterodimer (as K61 and C63 of CHIKV E1 domain II also bind the CDRs of CHIK-263). The ‘arch 2’ cleft of the E2 protein β-ribbon connector into which the CDRH3 of EEEV-373 inserts is stabilized by the extensive interactions with the underlying E1 glycoprotein. It is this interaction that renders the epitope conformational and quaternary, which is validated by the ELISA binding data showing that bivalent EEEV-373 does not bind either recombinant monomeric E2 or E1 glycoproteins but binds to the E2/E1 heterodimer and VLPs (FIG. 2A).

The bivalency requirement for complex formation is a remarkable feature of EEEV-373. Monovalent Fab molecules of neutralizing antiviral antibodies tend to have lower affinity to viruses compared to their bivalent counterpart due to the absence of avidity effects. However, the weak detectable binding observed for monovalent EEEV-373 Fab molecules is consistent with the requirement for bivalency to neutralize EEEV. The biphasic nature of association observed in the kinetic analyses strongly supports the contribution of avidity effects towards complex formation. The dependence on avidity may stem from the flexibility of the heterodimer epitope, necessitating slower interactions for stable complex formation. Given the lack of significant secondary structural elements in the β-ribbon connector, the ‘arch 2’ cleft may undergo structural transitions, leading to flexibility in the epitope. However, this could not be visualized due to the limited resolution of this region. Another explanation may result from binding of one Fab arm of the IgG molecule to the heterodimer epitope during the fast initial binding interaction step, while the other Fab arm transiently samples additional heterodimer epitopes in a slower fashion until recognizing the correct orientation to generate a high affinity complex. In such scenarios, avidity contributions from bivalent engagement of the IgG with virus particles shifts the equilibrium towards a stable complex formation.

The relatively high potency and intra-virion cross-linking displayed by EEEV-373 mirrors the activity of another potently neutralizing anti-EEEV antibody, EEEV-106⁴⁰. The ability to form intra-virion crosslinks on the icosahedral viral surface appears to correspond well with the high potency of neutralizing antibodies. The intra-virion cross-linking observed for EEEV-373 appears to be structurally constrained as the binding mode of EEEV-373 observed in the reconstruction strongly suggests that there is limited flexibility of the Fab arms with respect to one another. Theoretically, there are six possible ways the spikes across the i2 axis can be cross-linked by EEEV-373 (FIGS. 6A-G). As discussed, three of these binding modes (i3.1-q3.1, q3.1-q3.4, or q3.3-q3.4) are not feasible due to distance or steric constraints. However, while the remaining three modes (13.1-i3.2, q3.1-q3.3, and q3.1-i3.2) satisfy both conditions, only one binding orientation was observed (q3.1-93.3). In the i3.1-i3.2 binding mode, the Fab arms must stretch by ˜50% to simultaneously bind (FIG. 6D). In the q3.1-13.2 binding mode, IgG binding would require the Fab arms to rotate around the IgG 2-fold axis by ˜60° with respect to each other (FIG. 6F). It is not obvious from the structure why these two alternative binding modes are not observed. It may be that EEEV-373 transiently interacts with these orientations prior to binding the observed preferred orientation (q3.1-q3.3). This outcome may result from an energetically favorable interaction with the q3.1-93.3 binding mode.

EEEV-373 neutralizes EEEV by acting at multiple steps in the viral entry pathway (FIGS. 7A-F). EEEV-373 reduced ApoER2 or VLDLR binding by 80 or 50%, respectively, which suggests EEEV-373 binds near the receptor binding site or may sterically hinder the virus particles from binding to receptors (FIG. 11B). The incomplete extent of receptor blockade may be attributed to the exposure of additional epitopes besides those that are occupied by EEEV-373 IgG1 molecules. The receptor binding site for ApoER2 and VLDLR is thought to overlap on virus particles, as soluble VLDLR blocks EEEV infection of K562 cells overexpressing ApoER2²⁸. The observed differences in receptor blockade by EEEV-373 may be due to differences in binding affinity to virus particles. The apparent affinity of VLDLR to EEEV VLPs is ˜2.1 to 15 nM^{29,31}, whereas EEEV-373 binds to EEEV VLPs with an affinity of ˜14 nM. The strong affinity of VLDLR to EEEV VLPs may enable the receptor to outcompete EEEV-373, which may explain the incomplete blockade of VLDLR. In contrast, the affinity of ApoER2 to EEEV VLPs is not known. However, based on the results, this interaction may have lower affinity due to greater blockade of ApoER2 by EEEV-373.

Even after SINV/EEEV attachment to cells, EEEV-373 maintains neutralization activity, albeit at a ˜8-fold reduced potency. Post-attachment neutralization activity suggests EEEV-373 also inhibits viral entry into cells. Furthermore, bivalent EEEV-373 molecules (IgG or F(ab′)₂) inhibit early fusion events in the endosome, as suggested by its ability to prevent low pH-induced fusion of SINV/EEEV with liposomes. Altogether, EEEV-373 may block or partially stall steps necessary for virus entry and fusion into the cell. The cumulative inhibitory effect of EEEV-373 at multiple steps probably explains the observed potent neutralization of SINV/EEEV and 100% protective efficacy in a mouse subcutaneous challenge model of EEEV (FIGS. 1A-E). The bivalent requirement for EEEV-373 to neutralize SINV/EEEV entry may relate to how EEEV-373 binds to virus particles. The intra-virion cross-linking of the q3 spikes across the i2 axis suggests that SINV/EEEV particles may be stabilized, such that the necessary transitional changes for virus entry and fusion are hindered. The weak association and lack of neutralization activity against SINV/EEEV as Fab molecules supports this due to the absence of avidity effects.

EEEV-373 IgG1 molecules did not block viral egress from infected cells, and the antibody minimally binds to E2 or E1 proteins expressed at the plasma membrane (FIG. 15). These findings strongly suggest that the epitope is not assembled in the proper quaternary formation or is inaccessible during virus particle assembly and egress from the plasma membrane of infected cells. The structural arrangement of glycoproteins on intact virions presents the correct geometry (i.e., distance, angle, and curvature) for EEEV-373 to engage with its epitope, which is otherwise transient or absent on the plasma membrane of transiently transfected or infected cells during budding of virus progeny.

EEEV-373 is interesting because of its recognition of a complex dynamic antigenic site. The occurrence of bivalent interactions required for stable binding and efficient neutralization allows the antibody to perform multiple functions with less molecules. Complex epitopes on viral surface antigens are attractive vaccine targets since the neutralizing antibodies that target them tend to be extremely potent. Additionally, antibodies targeting complex conserved epitopes may provide broad protection against different serotypes or lineages of viruses. For example, for flaviviruses, such as dengue (DENV), antibodies that target quaternary epitopes within the E protein dimers show potent neutralization capability against all four DENV serotypes⁴⁷. Similarly, another potently neutralizing antibody currently in clinical development, designated ZIKV-117, neutralizes Zika virus strains from different lineages and targets a quaternary epitope spanning across E protein dimer-dimer interfaces^{48,49}. In contrast to flaviviruses like dengue, Zika and West Nile viruses, the structural characterization of quaternary epitopes on alphaviruses is lacking. Interestingly, cryo-EM reconstructions of alphaviruses in complex with protein receptors have shown the receptor binding site to interact with residues in both the E2 and E1 glycoproteins, thus, rendering alphavirus receptor binding a quaternary interaction^{24,26,29,31}. To the best of the inventors' knowledge, structural characterization of a highly conformationally-dependent epitope on the E2 glycoprotein of a major alphaviral pathogen targeted by a patient-derived human antibody has not been previously observed. The strict dependence on bivalency for binding and neutralization displayed by EEEV-373 highlights surprising intricacies of the antigen-antibody interaction. The elucidation of this complex interaction has important implications for therapeutic antibody developments against viral pathogens. Altogether, the work described here helps inform us on the rational design of immunogens for EEEV and likely other clinically relevant alphaviruses to elicit protective responses to complex epitopes.

TABLE A
Parameters for cryo-EM data collection and statistics of data processing,
refinement, and model validation of the SINV/EEEV + EEEV-373 IgG complex

SINV/EEEV + EEEV-373 IgG

| | Icosahedral | Localized |
|---|---|---|
| Parameters | reconstruction | reconstruction |
| Data collection and | | |
| processing | | |
| Nominal magnification | 64,000 | — |
| Voltage (kV) | 300 | — |
| Total electron dose (e⁻/Å²) | 35.49 | — |
| No. of frames per movie | 40 | — |
| Exposure time (s) | 3.12 | — |
| Frame exposure time (ms) | 78 | — |
| Dose rate (e⁻/Å²/s) | 11.38 | — |
| Defocus range (μm) | 1-3 | — |
| Pixel size (Å) | 0.66 | — |
| No. of micrographs collected | 1,276 | — |
| Number of particles selected | 17,830 | — |
| after 2D classification | | |
| Number of particles after 3D | 15,932 | 271,460 |
| classification for final | | |
| refinement | | |
| Symmetry imposed | Icosahedral | C1 |
| Map resolution (Å) (FSC = | 4.6 | 3.8 |
| 0.143) | | |
| Unmasked | 5.6 | 4.5 |
| Masked | 4.6 | 3.8 |
| Low pass filter resolution (Å) | 15.0 | 4.0 |
| Model fitting | | |
| Model used | E1-E2 heterodimer | E1-E2 heterodimer |
| | (PDB: 6MX4), Fab: | (PDB: 6MX4), Fab: |
| | Homology | Homology |
| | model (AlphaFold 3). | model (AlphaFold 3). |
| Fitting method and refinement | Rigid body fitting in | Rigid body fitting in |
| | ChimeraX followed by | ChimeraX followed by |
| | refinement in Phenix | refinement in Phenix |
| EMDB and PDB codes | EMD-43980 | EMD-43507 |
| | PDB ID: 9AY1 | PDB ID: 8VSV |
| Model validation | | |
| Number of chains | 18 | 28 |
| MolProbity score | 2.82 | 2.79 |
| Clash score | 8.31 | 7.79 |
| Ramachandran plot (%) | 90.46 | 90.27 |
| Favored | | |
| Ramachandran plot (%) | 9.28 | 9.38 |
| Allowed | | |
| Ramachandran plot (%) | 0.27 | 0.36 |
| Outliers | | |
| CC (mask) | 0.74 | 0.83 |
| CC (volume) | 0.71 | 0.81 |

TABLE 1
NUCLEOTIDE SEQUENCES FOR ANTIBODY VARIABLE REGION

| Clone | Seq ID | Chain | Variable Sequence Region |
|---|---|---|---|
| EEEV-373 | SEQ ID | heavy | CAGGTGCAGCTGGTGGAGTCTGGGGGAGGCGTGGTC |
| | NO: 1 | | CAGCCTGGGAGGTCCCTGAGACTCTCCTGTGCAGCC |
| | | | TCTGGATTCACCTTCAGTAGTCATGTTATGTACTGG |
| | | | GTCCGCCAGGCTCCAGGCAAGGGGCTGGAGTGGGTG |
| | | | GCAGTTATAACATATGATGGAGGCAATAAATACTAC |
| | | | GCAGACTCCGTGAGGGGCCGACTCACCATCTCCAGA |
| | | | GACAATTCCAAGAACACACTGTATCTGCAAATGAAC |
| | | | AGCCTGAGAGCTGAGGACACGGCTGTGTATTACTGT |
| | | | GCGAGTCCCCGTGGGGATAGTGGGAGCTACTACGAT |
| | | | ATAGACTACTTTGACTACIGGGGCCAGGGAACCCTG |
| | | | GTCACCGTCTCCTCG |
| | SEQ ID | light | GACATCCAGATGACCCAGTCTCCATCTGCCATGTCT |
| | NO: 2 | | GCATCTGTAGGAGACAGAGTCACCATCACTTGTCGG |
| | | | GCGAGTCAGGGCATTAGCAATTATTTAGCCTGGTTT |
| | | | CAGCAGAAACCAGGGAAAGTCCCTAAGCGCCTGATC |
| | | | TATGCTGCATCCAGTTTGCAAAGTGGGGTCCCATCA |
| | | | AGGTTCAGCGGCAGTGGATCTGGGACAGAATTCACT |
| | | | CTCACAATCAGCAGCCTGCAGCCTGAAGATTTTGCA |
| | | | ACTTATTACTGTCTACAGCATAATACTTCCCCTTCC |
| | | | CCGTTCGGCCAAGGGACCAAGGTGGAAATCAAA |

TABLE 2
PROTEIN SEQUENCES FOR ANTIBODY VARIABLE
REGION

| Clone | Seq ID | Chain | Variable Sequence Region |
|---|---|---|---|
| EEEV- | SEQ ID | heavy | QVQLVESGGGVVQPGRSL |
| 373 | NO: 3 | | RLSCAASGFTFSSHVMYW |
| | | | VRQAPGKGLEWVAVITYD |
| | | | GGNKYYADSVRGRLTISR |
| | | | DNSKNTLYLQMNSLRAED |
| | | | TAVYYCASPRGDSGSYYD |
| | | | IDYFDYWGQGTLVTVSS |
| | SEQ ID | light | DIQMTQSPSAMSASVGDR |
| | NO: 4 | | VTITCRASQGISNYLAWF |
| | | | QQKPGKVPKRLIYAASSL |
| | | | QSGVPSRFSGSGSGTEFT |
| | | | LTISSLQPEDFATYYCLQ |
| | | | HNTSPSPFGQGTKVEIK |

TABLE 3
HEAVY CHAIN SEQUENCES

| Clone | CDRH1 | CDRH2 | CDRH3 |
|---|---|---|---|
| EEEV- | GFTF | ITYD | ASPRGDSGS |
| 373 | SSHV | GGNK | YYDIDYFDY |
| | SEQ ID | SEQ ID | SEQ ID |
| | NO: 5 | NO: 6 | NO: 76 |

TABLE 4
LIGHT CHAIN SEQUENCES

| Clone | CDRL1 | CDRL2 | CDRL3 |
|---|---|---|---|
| EEEV- | QGISNY | AAS | LQHNTSPSP |
| 373 | SEQ ID | SEQ ID | SEQ ID |
| | NO: 8 | NO: 9 | NO: 10 |

All of the compositions and methods disclosed and claimed herein can be made and executed without undue experimentation in light of the present disclosure. While the compositions and methods of this disclosure have been described in terms of preferred embodiments, it will be apparent to those of skill in the art that variations may be applied to the compositions and methods and in the steps or in the sequence of steps of the method described herein without departing from the concept, spirit and scope of the disclosure. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the disclosure as defined by the appended claims.

### VII. References

The following references, to the extent that they provide exemplary procedural or other details supplementary to those set forth herein, are specifically incorporated herein by reference.
U.S. Pat. No. 3,817,837
U.S. Pat. No. 3,850,752
U.S. Pat. No. 3,939,350
U.S. Pat. No. 3,996,345
U.S. Pat. No. 4,196,265
U.S. Pat. No. 4,275,149
U.S. Pat. No. 4,277,437
U.S. Pat. No. 4,366,241
U.S. Pat. No. 4,472,509
U.S. Pat. No. 4,554,101
U.S. Pat. No. 4,680,338
U.S. Pat. No. 4,816,567
U.S. Pat. No. 4,867,973
U.S. Pat. No. 4,938,948
U.S. Pat. No. 5,021,236
U.S. Pat. No. 5,141,648
U.S. Pat. No. 5,196,066
U.S. Pat. No. 5,563,250
U.S. Pat. No. 5,565,332
U.S. Pat. No. 5,856,456
U.S. Pat. No. 5,880,270
U.S. Pat. No. 6,485,982
“Antibodies: A Laboratory Manual,” Cold Spring Harbor Press, Cold Spring Harbor, NY, 1988
Abbondanzo et al., Am. J. Pediatr. Hematol. Oncol., 12 (4), 480-489, 1990
Allred et al., Arch. Surg., 125 (1), 107-113, 1990
Atherton et al., Biol. of Reproduction, 32, 155-171, 1985
Barzon et al., Euro Surveill. 21 (32), 2016
Beltramello et al., Cell Host Microbe 8, 271-283, 2010
Brown et al., J. Immunol. Meth., 12; 130 (1),: 111-121, 1990
Campbell, In: Monoclonal Antibody Technology, Laboratory Techniques in Biochemistry and Molecular Biology, Vol. 13, Burden and Von Knippenberg, Eds. pp. 75-83, Amsterdam, Elsevier, 1984
Capaldi et al., Biochem. Biophys. Res. Comm., 74 (2): 425-433, 1977
De Jager et al., Semin. Nucl. Med. 23 (2), 165-179, 1993
Dholakia et al., J. Biol. Chem., 264, 20638-20642, 1989
Diamond et al., J Virol 77, 2578-2586, 2003
Doolittle and Ben-Zeev, Methods Mol. Biol., 109: 215-237, 1999
Duffy et al., N. Engl. J. Med. 360, 2536-2543, 2009
Elder et al., Infections, infertility and assisted reproduction. Part II: Infections in reproductive medicine & Part III: Infections and the assisted reproductive laboratory. Cambridge UK: Cambridge University Press; 2005
Gefter et al., Somatic Cell Genet., 3:231-236, 1977
Gornet et al., Semin Reprod Med. 34 (5): 285-292. 2016
Gulbis and Galand, Hum. Pathol. 24 (12), 1271-1285, 1993
Halfon et al., PLoS ONE 5 (5) e10569, 2010
Hessell et al., Nature 449, 101-4, 2007
Khatoon et al., Ann. of Neurology, 26, 210-219, 1989
King et al., J. Biol. Chem., 269, 10210-10218, 1989
Kohler and Milstein, Eur. J. Immunol., 6, 511-519, 1976
Kohler and Milstein, Nature, 256, 495-497, 1975
Kyte and Doolittle, J. Mol. Biol., 157 (1): 105-132, 1982
Mansuy et al., Lancet Infect Dis. 16 (10): 1106-7, 2016
Nakamura et al., In: Enzyme Immunoassays: Heterogeneous and Homogeneous Systems, Chapter 27, 1987
O'Shannessy et al., J. Immun. Meth., 99, 153-161, 1987
Persic et al., Gene 187:1, 1997
Potter and Haley, Meth. Enzymol., 91, 613-633, 1983
Purpura et al., Lancet Infect Dis. 16 (10): 1107-8, 2016
Remington's Pharmaceutical Sciences, 15th Ed., 3:624-652, 1990
Tang et al., J. Biol. Chem., 271:28324-28330, 1996
Wawrzynczak & Thorpe, In: Immunoconjugates, Antibody Conuugates In Radioimaging And Therapy Of Cancer, Vogel (Ed.), NY, Oxford University Press, 28, 1987
Yu et al., J Immunol Methods 336, 142-151, doi: 10.1016/j.jim.2008.04.008, 2008
1. Crowe, Annu Rev Immunol 40, 349-386 (2022).
2. Oliphant & Diamond, Expert Opin Biol Ther 7, 885-892 (2007).
3. Rey & Lok, Cell 172, 1319-1334 (2018).
4. Sevvana & Kuhn, Curr Opin Virol 45, 51-64 (2020).
5. Fox et al., Cell 163, 1095-1107 (2015).
6. Hasan et al., Cell Rep 25, 3136-3147.e5 (2018).
7. Jin & Simmons, Viruses 11, 305 (2019).
8. Zhang et al., Cell Rep 31, 107584 (2020).
9. Zhou et al., Proc Natl Acad Sci USA 117, 27637-27645 (2020).
10. Armstrong & Andreadis, N Engl J Med 368, 1670-1673 (2013).
11. Ayres & Feemster, N Engl J Med 240, 960-962 (1949).
12. Lindsey et al., Am J Trop Med Hyg 98, 1472-1477 (2018).
13. Lindsey et al., MMWR Morb Mortal Wkly Rep 69, 50-51 (2020).
14. Ronca et al., Front Microbiol 7, 959 (2016).
15. Sidwell & Smee, et al., Antiviral Res 57, 101-111 (2003).
16. Jose et al., Future Microbiol 4, 837-856 (2009).
17. Zhang et al., EMBO J 30, 3854-3863 (2011).
18. Button et al., Curr Opin Virol 45, 25-33 (2020).
19. Mukhopadhyay et al., Structure 14, 63-73 (2006).
20. Gibbons et al., J Virol 78, 3312-3318 (2004).
21. Holmes et al., PLoS Pathog 16, e1008876 (2020).
22. Voss et al., Nature 468, 709-712 (2010).
23. Zhang et al., Nature 557, 570-574 (2018).
24. Basore et al., Cell 177, 1725-1737.e16 (2019).
25. Ma et al., Nature 588, 308-314 (2020).
26. Basore et al., Nature 598, 672-676 (2021).
27. Chen et al., Proc. Natl. Acad. Sci. U.S.A. 117, 8890-8899 (2020).
28. Clark et al., Nature 602, 475-480 (2022).
29. Yang et al., Structural basis for VLDLR recognition by Eastern equine encephalitis virus. Preprint at: world-wide-web at biorxiv.org/content/10.1101/2023.11.14.567065v1.full.pdf.
30. Ma et al., Nat Commun 15, 246 (2024).
31. Adams et al., Cell S0092-8674, 01318-1 (2024).
32. Kielian, Annu Rev Virol 1, 171-189 (2014).
33. Li et al., Nature 468, 705-708 (2010).
34. Earnest et al., J Exp Med 216, 2282-2301 (2019).
35. Jin et al., Cell Rep 13, 2553-2564 (2015).
36. Jin et al., Cell Host Microbe 24, 417-428.e5 (2018).
37. Long et al., Proc Natl Acad Sci USA 112, 13898-13903 (2015).
38. Powell et al., Cell Host Microbe 28, 699-711.e7 (2020).
39. Williamson et al., Cell 183, 1884-1900.e23 (2020).
40. Williamson et al., Proc Natl Acad Sci USA 120, e22131690120 (2023).
41. Kim et al., Cell 184, 4414-4429.e19 (2021).
42. Williamson et al., Cell 184, 4430-4446.e22 (2021).
43. Kim et al., Nat Microbiol 4, 187-197 (2019).
44. Fibriansah et al., Science 349, 88-91 (2015).
45. Sharma et al., Cell 184, 6052-6066.e18 (2021).
46. Chmielewski et al., Nat Microbiol 7, 1270-1279 (2022).
47. Dejnirattisai et al., Nat Immunol 16, 170-177 (2015).
48. Hasan et al., Nat Commun 8, 14722 (2017).
49. Sapparapu et al., Nature 540, 443-447 (2016).
50. Gardner et al., Proc Natl Acad Sci USA 108, 16026-31 (2011).
51. Ko et al., Sci Transl Med 11, eaav3113 (2019).
52. Kafai et al., J Exp Med 219, e20212532 (2022).
53. Scheres & Chen, Nat Methods 9, 853-854 (2012).
54. de la Rosa-Trevín et al., J Struct Biol 195, 93-99 (2016).
55. Zivanov et al., Elife 7, e42166 (2018).
56. Liebschner et al., Acta Crystallogr D Struct Biol 75, 861-877 (2019).
57. Pettersen et al., Protein Sci 30. 70-82 (2021).
58. Croll. Acta Crystallogr D Struct Biol 74. 519-530 (2018).
59. Emsley & Cowtan. Acta Crystallogr D Biol Crystallogr 60. 2126-2132 (2004).
60. Williams & Barclay. Annu Rev Immunol 6. 381-405 (1988).

1. A method of detecting an Eastern Equine Encephalitis Virus (EEEV) infection in a subject comprising:
(a) contacting a sample from said subject with an antibody or antibody fragment having clone-paired heavy and light chain CDR sequences from Tables 3 and 4, respectively; and
(b) detecting EEEV in said sample by binding of said antibody or antibody fragment to an EEEV antigen in said sample.

2.-12. (canceled)

13. A method of treating a subject infected with Eastern Equine Encephalitis Virus (EEEV), or reducing the likelihood of infection of a subject at risk of contracting EEEV, comprising delivering to said subject an antibody or antibody fragment having clone-paired heavy and light chain CDR sequences from Tables 3 and 4, respectively.

14. The method of claim 13, the antibody or antibody fragment is encoded by clone-paired light and heavy chain variable sequences as set forth in Table 1.

15. The method of claim 13, the antibody or antibody fragment is encoded by clone-paired light and heavy chain variable sequences having 95% identity to as set forth in Table 1.

16. The method of claim 13, wherein said antibody or antibody fragment is encoded by light and heavy chain variable sequences having 70%, 80%, or 90% identity to clone-paired sequences from Table 1.

17. The method of claim 13, wherein said antibody or antibody fragment comprises light and heavy chain variable sequences according to clone-paired sequences from Table 2.

18. The method of claim 13, wherein said antibody or antibody fragment comprises light and heavy chain variable sequences having 70%, 80% or 90% identity to clone-paired sequences from Table 2.

19. The method of claim 13, wherein said antibody or antibody fragment comprises light and heavy chain variable sequences having 95% identity to clone-paired sequences from Table 2.

20. The method of claim 13, wherein the antibody fragment is a recombinant scFv (single chain fragment variable) antibody, Fab fragment, F(ab′)₂ fragment, or Fv fragment.

21. The method of claim 13, wherein said antibody is an IgG, or a recombinant IgG antibody or antibody fragment comprising an Fc portion mutated to alter (eliminate or enhance) FcR interactions, to increase half-life and/or increase therapeutic efficacy, such as a LALA, N297, GASD/ALIE, YTE or LS mutation or glycan modified to alter (eliminate or enhance) FcR interactions such as enzymatic or chemical addition or removal of glycans or expression in a cell line engineered with a defined glycosylating pattern.

22. The method of claim 13, wherein said antibody is a chimeric antibody or a bispecific antibody.

23. The method of claim 13, wherein said antibody or antibody fragment is administered prior to infection or after infection.

24. The method of claim 13, wherein said subject is a pregnant female, a sexually active female, or a female undergoing fertility treatments.

25. The method of claim 13, wherein delivering comprises antibody or antibody fragment administration, or genetic delivery with an RNA or DNA sequence or vector encoding the antibody or antibody fragment.

26. A monoclonal antibody, wherein the antibody or antibody fragment comprising clone-paired heavy and light chain CDR sequences from Tables 3 and 4, respectively, or a hybridoma or an engineered cell expressing the same.

27.-46. (canceled)

47. A vaccine formulation comprising one or more antibodies or antibody fragments or expression vector(s) encoding the same, comprising clone-paired heavy and light chain CDR sequences from Tables 3 and 4, respectively.

48.-96. (canceled)

97. A human monoclonal antibody or antibody fragment, or hybridoma or engineered cell producing the same, wherein said antibody binds to Eastern Equine Encephalitis Virus (EEEV) E1 protein and either (a) binds to but does not neutralize EEEV or (b) binds to EEEV E1 protein and neutralizes EEEV.
